(19) United States
(12) Patent Application Publication
(10) Pub. No.: US 2023/0352117 A1
(43) Pub. Date: Nov. 2, 2023

(54) SYSTEMS AND METHODS FOR ANALYSIS OF PRESENCE OF MICROORGANISMS

(71) Applicant: IDbyDNA Inc., Salt Lake City, UT (US)

(72) Inventors: Robert Schlaberg, Salt Lake City, UT (US); Steven Flygare, Salt Lake City, UT (US); Kate Broadbent, Salt Lake City, UT (US); Qing Li, Salt Lake City, UT (US); Hajime Matsuzaki, Salt Lake City, UT (US); Wan Xie, Salt Lake City, UT (US)

(21) Appl. No.: 18/003,492

(22) PCT Filed: Feb. 23, 2022
(86) PCT No.: PCT/US2022/017523

§ 371 Date: Dec. 27, 2022

Related U.S. Application Data
(60) Provisional application No. 63/152,765, filed on Feb. 23, 2021.

(51) Int. Cl. G16B 30/00; C12Q 1/6888; G16B 45/00; G16H 50/20

(52) U.S. Cl. CPC G16B 30/00; C12Q 1/6888; G16B 45/00; G16H 50/20; C12Q 2600/156

(57) ABSTRACT

A method for reviewing sequencing data for identifying microorganisms in a sample is provided. A request to display an analysis of a result set obtained from sequencing nucleic acids from the sample is received. The result set includes sequencing statistics, nucleotide sequences mapped against microorganism reference sequences, and mapping statistics for the mapping of nucleotide sequences to each respective reference sequence. A first customizable diagnostic template is applied to the result set. The customizable diagnostic template specifies a subset of sequencing statistics, a subset of microorganisms, and a subset of mapping statistics. A customizable user interface is displayed comprising a. review' status, a. first affordance for updating the review' status, a summary of the subset of sequencing statistics, a summary of the subset of mapping statistics for each microorganism satisfying a minimum mapping threshold, and a second affordance for applying a second customizable diagnostic template to the result set.

### CROSS REFERENCE TO RELATED APPLICATIONS

This patent application claims priority to U.S. Provisional Patent Application No. 63/152,765 entitled “Systems and Methods for Analysis of Presence of Microorganisms,” filed Feb. 23, 2021, which is hereby incorporated by reference in its entirety.

### TECHNICAL FIELD

This specification describes technologies relating to visualizing sequencing information.

### BACKGROUND

Metagenomics, the genomic analysis of a population of microorganisms, makes possible the profiling of microbial communities in the environment and the human body at unprecedented depth and breadth. Its rapidly expanding use is revolutionizing our understanding of microbial diversity in natural and man-made environments and is linking microbial community profiles with health and disease. To date, most studies have relied on PCR amplification of microbial marker genes (e.g., bacterial 16S rRNA), for which large, curated databases have been established. More recently, higher throughput and lower cost sequencing technologies have enabled a shift towards enrichment-independent or broad pathogen enrichment-based next-generation sequencing (NGS) to profile microbial and host markers and their influence on health and infectious and other diseases (jointly referred to as “NGS ID”). These approaches reduce bias, improve detection of less abundant taxa, and enable discovery of novel pathogens and expression of genes of interest.

While conventional pathogen-specific nucleic acid amplification tests are highly sensitive and specific, they require a priori knowledge of likely pathogens, as with limited diagnostic panels to enable diagnosis of the most common pathogens. In contrast, NGS ID allows for unbiased detection and molecular typing of a theoretically unlimited number of common and unusual pathogens, as well as Antimicrobial Resistance (AMR) markers. Wide availability of next-generation sequencing instruments, lower reagent costs, and streamlined sample preparation protocols are enabling an increasing number of investigators to perform high-throughput DNA and RNA-seq for metagenomics studies. However, analysis of sequencing data is still formidably difficult and time consuming, requiring bioinformatics skills, computational resources, and microbiological expertise that is not available to many laboratories and/or practitioners, especially diagnostic ones.

### SUMMARY

Technical solutions (e.g., computing systems, methods, and non-transitory computer readable storage mediums) for addressing the above identified problems with discovering patterns in data sets are provided in the present disclosure.

As discussed above, next-generation sequencing techniques generate a large amount of sequencing data that can be prohibitively complex for a practitioner in a clinical or laboratory setting to review efficiently in order to provide informed decisions for further action (e.g., a treatment regimen for a patient based one or more pathogens identified in a patient sample). Thus, there is a need in the art for systems and methods that allow for the presentation and visualization of sequencing data to detect and analyze microorganisms and their hosts in biological and/or non-biological samples, without requiring the practitioner to possess advanced genomics, bioinformatics, and statistical skills, as well as microbiological expertise across a wide array of clades and classes.

The present disclosure provides a comprehensive approach to identification and analysis of organisms (e.g., microorganisms, pathogens and/or AMR markers) and their hosts in a biological and/or non-biological sample, such as a sample obtained from a patient. For example, sequencing data obtained from the patient sample is entered into an analysis pipeline comprising mapping (e.g., alignment) to one or more reference sequences corresponding to a set of microorganisms (e.g., complete and/or incomplete genomes for the set of microorganisms), thus generating preliminary results including the number and identity of microorganisms in the sample, quality control data, and/or sequencing metadata (e.g., number of reads, coverage, and/or alignment identity). Systems and methods for visualizing and reviewing the results obtained from the analysis pipeline allows users in clinical or laboratory settings to quickly and efficiently analyze the biological and/or non-biological sample, allowing the transmission of relevant results for further action (e.g., for diagnosis, monitoring, treatment, or regulatory purposes). For example, the transmission of relevant results and/or any recommended actions can be provided in a report following approval of the preliminary results by a medical practitioner.

The systems and methods disclosed herein provide a user or practitioner with access to information that is used for downstream decision-making (e.g., for the issuance of a report), while allowing flexibility for a streamlined or detailed analysis approach. For example, the interactive visualization and review tools provided herein are optionally automated, thus avoiding the need for the practitioner to have extensive bioinformatics and/or microbiological expertise to generate actionable results based on sequencing data. Alternatively, in some instances, the interactive visualization and review tools provided herein are customizable, thus allowing additional interaction for troubleshooting, pipeline development, or directing analysis towards specific organisms of interest (e.g., by application of filters). Generally, a minimum of user interaction is employed for final approval of the relevant results, whether using the streamlined or the detailed analysis approach.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

One aspect of the present disclosure provides a method for facilitating review of nucleic acid sequencing data prepared for identifying the presence of a subset of microorganisms and/or antimicrobial resistance markers in a biological or non-biological sample (e.g., from a subject), at a computer system having a display, one or more processors, and memory storing one or more programs for execution by the one or more processors.

The method includes receiving a request to display an analysis of a result set obtained from a sequencing reaction of nucleic acids from the biological and/or non-biological sample. The result set includes a plurality of sequencing statistics from the sequencing reaction, a plurality of nucleotide sequences mapped against a plurality of reference sequences corresponding to a set of microorganisms, where the set of microorganisms comprises at least 3, at least 5, at least 10, at least 20, at least 30, at least 40, at least 50, at least 60, at least 70, at least 80, at least 90, or at least 100 microorganisms, and for each respective microorganism in the set of microorganisms, a corresponding plurality of mapping statistics for the mapping of respective nucleotide sequences to the reference sequence for the respective microorganism or hosts.

Responsive to the request, a first customizable diagnostic template is applied to the result set, where the customizable diagnostic template specifies a subset of the plurality of sequencing statistics, a subset of the set of microorganisms, and a subset of the plurality of mapping statistics.

The method further includes displaying, on the display, a customizable user interface comprising a review status for the nucleic acid sequencing data, a first affordance for updating the review status for the nucleic acid sequencing data, a summary of the subset of the plurality of sequencing statistics, for each respective microorganism in the subset of the set of microorganisms satisfying a minimum mapping threshold in the result set, a corresponding summary of the subset of the plurality of mapping statistics for the respective nucleotide sequences in the plurality of nucleotide sequences mapped to the reference sequence for the respective microorganism, and a second affordance for applying a second customizable diagnostic template to the result set.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is an example block diagram illustrating a computing device and related data structures used by the computing device in accordance with some implementations of the present disclosure.

FIGS. 2A and 2B collectively illustrate an example method in accordance with an embodiment of the present disclosure, in which optional steps are indicated by broken lines.

FIG. 3A illustrates a display of an index of samples (e.g., a sample queue on a dashboard of a review and visualization system) for review of nucleic acid sequencing data, in accordance with some embodiments of the present disclosure. FIG. 3B illustrates an affordance for modifying the display of the index of samples for review of nucleic acid sequencing data, in accordance with some embodiments of the present disclosure.

FIG. 4 illustrates a customizable user interface that displays an analysis of a result set obtained from a sequencing reaction of nucleic acids from a sample, in accordance with some embodiments of the present disclosure.

FIGS. 5A, 5B, 5C, and 5D collectively illustrate a display of a subset of a plurality of mapping statistics, in accordance with some embodiments of the present disclosure.

FIGS. 6A, 6B, 6C, 6D, 6E, 6F, and 6G collectively illustrate a display of a subset of a plurality of mapping statistics, in accordance with some embodiments of the present disclosure. FIG. 6G illustrates an overlay display of a mapping statistic, responsive to a user interaction, in accordance with an embodiment of the present disclosure.

FIG. 7 illustrates a customizable user interface that displays an analysis of a result set obtained from a sequencing reaction of nucleic acids from a positive control sample, in accordance with some embodiments of the present disclosure.

FIG. 8 illustrates a customizable user interface that displays an analysis of a result set obtained from a sequencing reaction of nucleic acids from a negative control sample, in accordance with some embodiments of the present disclosure.

FIG. 9 illustrates a customizable user interface that displays an analysis of a result set obtained from a sequencing reaction of nucleic acids from a blank control sample, in accordance with some embodiments of the present disclosure.

FIG. 10 illustrates a customizable user interface that displays an affordance for receiving a request to display an analysis of a result set obtained from a sequencing reaction of nucleic acids from a sample, in accordance with some embodiments of the present disclosure.

FIG. 11 illustrates an affordance for customizing a display of a summary of each microorganism in a set of microorganisms, where the set of microorganisms comprises at least 3, at least 5, at least 10, at least 20, at least 30, at least 40, at least 50, at least 60, at least 70, at least 80, at least 90, or at least 100 microorganisms, in accordance with some embodiments of the present disclosure.

FIG. 12 illustrates an affordance for applying a second customizable diagnostic template to a result set obtained from a sequencing reaction of nucleic acids from a sample, in accordance with some embodiments of the present disclosure.

FIG. 13 illustrates affordances for customizing the analysis of presence of microorganisms in a result set obtained from a sequencing reaction of nucleic acids from a sample, in accordance with some embodiments of the present disclosure.

FIG. 14 illustrates an affordance for adding a microorganism to a set of microorganisms, in accordance with some embodiments of the present disclosure.

FIGS. 15A, 15B. 15C, and 15D illustrate affordances for customizing the analysis of presence of microorganisms in a result set obtained from a sequencing reaction of nucleic acids from a sample, in accordance with some embodiments of the present disclosure.

FIGS. 16A and 16B illustrate affordances for customizing the analysis of presence of microorganisms in a result set obtained from a sequencing reaction of nucleic acids from a sample, in accordance with some embodiments of the present disclosure.

FIGS. 17A, 17B, 17C, 17D, 17E, 17F, 17G, and 17H collectively illustrate an example report generated using an analysis of presence of microorganisms in a result set obtained from a sequencing reaction of nucleic acids from a sample, in accordance with some embodiments of the present disclosure.

FIGS. 18A and 18B illustrate a customizable user interface that displays a plurality of sequencing quality control metrics, in accordance with some embodiments of the present disclosure.

FIGS. 19A and 19B illustrate a customizable user interface that displays a plurality of sample quality control metrics, in accordance with some embodiments of the present disclosure.

FIGS. 20A and 20B illustrate a customizable user interface that displays a plurality of batch quality control metrics, in accordance with some embodiments of the present disclosure.

FIG. 21 illustrates a customizable user interface comprising a dashboard for an administrator user account, in accordance with some embodiments of the present disclosure.

FIGS. 22A and 22B illustrate a customizable user interface comprising an index of sample reports, in accordance with some embodiments of the present disclosure.

FIGS. 23A, 23B, 23C, 23D, 23E, 23F, 23G, 23H, and 23I illustrate a customizable user interface for managing a second customizable diagnostic template, in accordance with some embodiments of the present disclosure.

FIG. 24 illustrates a customizable user interface for managing a plurality of users of a method for facilitating review of nucleic acid sequencing data prepared for identifying the presence of a subset of microorganisms and/or antimicrobial resistance markers in a sample (e.g., from a subject), in accordance with some embodiments of the present disclosure.

FIGS. 25A and 25B illustrate a customizable user interface for managing subsets of a plurality of users of a method for facilitating review of nucleic acid sequencing data prepared for identifying the presence of a subset of microorganisms and/or antimicrobial resistance markers in a sample (e.g., from a subject), in accordance with some embodiments of the present disclosure.

FIG. 26 illustrates a customizable user interface for managing a plurality of users of a method for facilitating review of nucleic acid sequencing data prepared for identifying the presence of a subset of microorganisms and/or antimicrobial resistance markers in a sample (e.g., from a subject), in accordance with some embodiments of the present disclosure.

FIG. 27 illustrates a customizable user interface for managing a method for facilitating review of nucleic acid sequencing data prepared for identifying the presence of a subset of microorganisms and/or antimicrobial resistance markers in a sample (e.g., from a subject), in accordance with some embodiments of the present disclosure.

FIG. 28 illustrates a display of an index of samples (e.g., a result history of a review and visualization system) for review of nucleic acid sequencing data, in accordance with some embodiments of the present disclosure.

FIG. 29 illustrates an example workflow of a method in accordance with some embodiments of the present disclosure.

### DETAILED DESCRIPTION

### Introduction

Infectious disease testing can be achieved using metagenomics, the detection and genomic analysis of a population of microorganisms (e.g., pathogens) and their hosts in a biological and/or non-biological sample. In combination with next-generation sequencing techniques (e.g., NGS ID), metagenomics facilitates such detection even without a priori knowledge of pathogens likely to be present in a sample. For example, in some instances, detection of microorganisms in biological and/or non-biological samples utilizes enrichment-based approaches comprising targeted enrichment panels, which provide increased depth and precision, reduce the occurrence of host or contaminant genetic material in the data set, and can be optimized for sequencing of specific regions. In some instances, detection of microorganisms utilizes enrichment-independent approaches, which provides increased breadth and resolution and can be used to identify both known and unknown microorganisms, including rare microorganisms. Generally, the detection of microorganisms using NGS ID can be used for numerous downstream actions including results reporting, patient diagnosis, treatment, and monitoring, analysis pipeline validation, and/or regulatory purposes.

In some instances, analysis of metagenomics data obtained by next-generation sequencing (e.g., whole-genome sequencing) involves a level of training (e.g., in bioinformatics, genomics, statistics, and microbiology) that many clinical and laboratory practitioners lack. In particular, for applications where the desired output is an actionable result, such as an identity of a pathogenic microorganism for a patient diagnosis or a presence of an AMR marker (e.g., an AMR gene) to determine whether a specific treatment is preferable over another, it can be impractical as well as inefficient for the practitioner to exhaustively analyze the entirety of the sequencing and/or mapping (e.g., alignment) data generated using NGS ID. In some embodiments, the ability to efficiently and accurately identify AMR markers improves treatment of microbial infections by indicating whether a particular microorganism is likely to respond to a course of therapy. See, for example, Greninger (2018). “The challenge of diagnostic metagenomics,” Expert Rev Mol Diagn 18:7, 605-615. doi:10.1080/14737159.2018.1487292.

Conversely, NGS ID approaches frequently suffer from a lack of understanding of true clinical utility, such as in instances where data-driven analyses are relied upon too heavily, without consideration of case-specific factors. For example, an accurate interpretation of sequencing and mapping data can be impacted by particularities specific to a patient, which may not be accounted for in an analysis pipeline. In some instances, additional benefit is obtained from further validation by a physician or medical practitioner in a clinical setting, and/or a laboratory inspector in a commercial or diagnostic setting. In some cases, additional oversight is used to account for contaminants common in wet-lab practices (e.g., clinical chemistry and/or PCR diagnostics), anomalies occurring in sequencing and/or mapping analysis (e.g., index hopping), and interference from host or nonpathogen nucleic acids, which can obfuscate the detection of pathogenic microorganisms of interest. This is especially important when distinguishing between two or more microbial populations in coinfections or detecting the presence of small populations of microorganisms, where even low levels of contaminating material can cause interference (e.g., due to the relative size of the microbial genomes compared to a host genome or a dominant population). A priori knowledge is useful, in some embodiments, for setting specific thresholds for the detection of microorganisms involved in certain pathogenic infections, where the limit of sensitivity of the sequencing reaction can differ based on the expected microbial populations in the sample. See, for example, Greninger (2018), “The challenge of diagnostic metagenomics,” Expert Rev Mol Diagn 18:7, 605-615, doi:10.1080/14737159.2018.1487292.

For example, in some instances, an understanding of the clinical relevance of a microorganism or AMR marker detected in a biological and/or non-biological sample is a key factor in determining whether it is actionable and thus whether it should be reported. While an automated approach can use machine learning approaches (e.g., string matching, regular expressions, natural language processing, etc.) to annotate and filter preliminary results based on published knowledge, in many situations, the analysis of microorganism detection benefits from a case-specific consideration. In such instances, conventional approaches that operate entirely without a priori knowledge may result in inaccurate interpretations of clinical data, compared to those that provide a mechanism for incorporating the same into the reporting of relevant results and the application of such results to downstream actions.

There is a need in the art for an approach to detection of microorganisms and AMR markers that will overcome the above limitations. In particular, the present disclosure provides systems and methods for analysis with or without review of the presence of microorganisms and/or antimicrobial resistance (AMR) markers in a biological and/or non-biological sample. The provided systems and methods utilize an automated approach that reduces the level of expertise and experience required to make accurate and reliable assessments based on the generated results, thus increasing accessibility and reducing the cost and labor required to train practitioners in the various skills and tools necessary for metagenomics sequencing analysis using NGS ID. For instance, as described below in Example 1, the streamlined example system and method (e.g., the ReviewPortal) provides a user interface that allows for a variety of display windows, dashboards, overlays, indexes, and other organizational features for the analysis of the result set, as well as multiple affordances for selection and customization of data and navigation between different display windows. Furthermore, the provided systems and methods improve workflow by streamlining the analysis and reporting process, thus reducing the amount of time and number of computational operations required to analyze each result set and increasing output (e.g., more samples can be processed, sequenced, analyzed and reported in a shorter time). Such reduction in computational time and complexity improves system operation and functionality, which can further reduce running time, save on power requirements, and improve user accessibility by allowing the analysis to be displayed with the relevant data at hand in fewer clicks compared to conventional systems and methods.

Additionally, the provided systems and methods allow for customization and/or validation of the sequencing data, mapping data, and analysis results, thus accounting for noisy data and ambiguous or inconclusive results. Such user interaction improves upon the prior art by facilitating the application of clinical oversight to the automated results based on, for example, a priori knowledge. Other benefits include increased consistency, where the streamlined reporting and analysis system can be uniformly performed based on predetermined parameters (e.g., one or more parameters saved as a filter or profile). By providing for at least a minimum amount of user interaction (e.g., final approval and/or safeguards requiring additional validation) and the ability to customize the analysis of the results set based on a priori knowledge or case-specific parameters (e.g., tailoring the presentation of information, filtering, and/or selection of sequencing or alignment metrics), the accuracy of the reported results can be improved.

Improved applicability of metagenomics sequencing analysis allows the practitioner to take advantage of additional benefits imparted by NGS ID. For example, the use of enrichment-independent metagenomics sequencing approaches increases the likelihood of detecting microorganisms that fail to be detected by other methods, such as conventional methods that rely on diagnostic panels limited to known and/or common pathogens. This ability to detect common and rare pathogens improves diagnostic applications, where the cause of a disease is unknown and diagnostic panels are unable to provide information as to the etiology of the disease or provide guidelines as to appropriate treatment. See, for example, Greninger (2018). “The challenge of diagnostic metagenomics,” Expert Rev Mol Diagn 18:7, 605-615, doi:10.1080/14737159.2018.1487292.

Additionally, the use of NGS ID reduces the likelihood of sample loss or degradation and increases the sensitivity of detection by, for example, eliminating the need for n vitro microbial culture. For instance, sample loss or degradation can occur through user error (e.g., by improper storage or handling of samples during sample collection, preparation or culture). Furthermore, a vast majority of microorganisms have not been adapted to in vitro culture, while other uncommon and/or novel microorganisms cannot be readily cultured. It is estimated that less than 1% of microorganisms present in the environment can be cultured in vitro. See, Streit and Schmitz (2004). “Metagenomics—the key to the uncultured microbes,” Curr Op Microb 7, 492-498. doi:10.1016/j.mib.2004.08.002. Loss of detectable microorganisms can also occur in hospital settings prior to sample collection, such as in instances where patients undergo a treatment (e.g., an antibiotic therapy) immediately after admission and initial diagnosis. In such cases, patient samples collected after antibiotic exposure may not be suitable for laboratory culture, and the subsequent detection of microorganisms may not be representative of the actual in vivo composition of pathogens. See. Harris et al., (2017), “Influence of Antibiotics on the Detection of Bacteria by Culture-Based and Culture-Independent Diagnostic Tests in Patients Hospitalized With Community-Acquired Pneumonia.” Open Forum Infect Dis 4(1), doi:10.1093/ofid/ofx014.

As sequencing costs drop. NGS ID operations can also be automated with significant price reductions. Large-scale sequencing technologies, such as next generation sequencing, have afforded the opportunity to achieve sequencing at costs that are less than one U.S. dollar per million bases, and, in fact, costs of less than ten U.S. cents per million bases have been realized. See. Nimwegen et al., (2016). “Is the $1000 Genome as Near as We Think? A Cost Analysis of Next-Generation Sequencing,” Clin Chem 62(11): 1458-1464, doi:10.1373/clinchem.2016.258632. The presently disclosed systems and methods therefore provide additional benefits by overcoming the limitations of using culture-based microbial diagnostic methods by allowing the use of an NGS ID approach instead of, or in addition to, an in vitro culture approach.

Moreover, the presently disclosed systems and methods provide a powerful tool that can be used to identify and detect microorganisms or antimicrobial resistance markers in a sample including large amounts of sequencing data, such as those obtained using NGS. Such systems and methods improve upon conventional systems and methods by facilitating analyses that are otherwise too complex to be performed in the human mind. For example, as described below, in some embodiments, the method includes receiving a request to display an analysis of a result set obtained from a sequencing reaction of nucleic acids from a sample, where the result set includes, at least, a plurality of nucleotide sequences, obtained from a sequencing reaction, mapped against a plurality of reference sequences corresponding to a set of microorganisms (e.g., at least 3 microorganisms). For an example analysis where the plurality of nucleotide sequences includes at least 1×10⁴ nucleotide sequences and the mapping the plurality of nucleotide sequences to the plurality of reference sequences collectively maps to at least 0.5 megabases (e.g., 500,000 base pairs), the number of calculations required to align each nucleotide sequence in the at least 1×10⁴ nucleotide sequences to each candidate position along the length of the collective 0.5 megabases and correctly assign any resulting mappings to the respective corresponding microorganism in the set of microorganisms, is so large that it cannot be performed mentally.

The following describes an example embodiment of a review and visualization tool for generating, viewing, modifying, validating, and/or reporting the results of a sequencing and mapping (e.g., alignment) analysis using nucleic acids in a biological or non-biological sample obtained (e.g., from a subject such as a patient). Briefly, a sample is collected, prepared, sequenced (e.g., by next-generation sequencing), and analyzed. In some embodiments, the analysis comprises preprocessing and/or pre-sorting of the sequencing data. Pre-sorting can include sorting each nucleotide sequence obtained from the sequencing of the sample into one or more bins, where each bin corresponds to a different microorganism, depending on the likelihood that the nucleotide sequence originated from the respective microorganism. Each nucleotide sequence is then mapped (e.g., using a k-mer alignment and/or a full alignment) to one or more reference sequences (e.g., complete and/or incomplete genomes) corresponding to different microorganisms. In some embodiments, the analysis is performed using an analysis pipeline.

The sequencing and mapping (e.g., alignment) data can then be accessed from the review and visualization tool, which can be a cloud-based interface such as an online portal. In some embodiments, one or more pending samples are displayed on the review and visualization tool (e.g., positive controls, negative controls, blank controls and/or analysis samples). In some embodiments, one or more batches, each including one or more pending samples, are displayed for individual review and visualization. Additional views are possible, including selection of different runs, each including one or more batches.

In some embodiments, selection of a sample generates an overview of the results set generated by, e.g., the analysis pipeline, indicating the number of microorganisms and/or antimicrobial resistance (AMR) markers, if any, detected in the sample. Detected microorganisms can be identified by scientific name, designated as pathogenic or nonpathogenic, annotated with various search terms, and/or categorized into various classes (e.g., bacteria, fungi, parasites, or viruses). Selection of each sample can also include presentation (e.g., in text or graphical form) of metadata, including sequencing statistics (e.g., nucleotide sequence count, base composition, sequencing library size, etc.), mapping statistics for each microorganism to which mapping was detected (e.g., coverage, sequence alignment score, consensus sequence, etc.), and/or run metrics (e.g., sample type, run accession number, review status, etc.). In some embodiments, additional information for one or more features are accessible through external links, including sequences for reference sequences (e.g., BLAST, NCBI) and/or databases for detected or otherwise selected microorganisms (e.g., Ensembl, EuPathDB, The Human Microbiome Project, Pathogen Portal, etc.). Generally, detection of microorganisms is performed using an automated process, using predefined thresholds for a plurality of parameters. However, these thresholds can be adjusted by a user or practitioner, as discussed below.

Selection of each sample can also include a display of quality control data, such as sequencing and mapping quality control data. For example, presentation of quality control data allows a user to assess whether a sequencing and/or mapping has been performed successfully before determining whether the output of the analysis is accurate and meaningful. Confirmation that control and analysis samples have passed quality control checks provides assurance that any subsequent analytical results and/or interpretations are reliable at least based on the performance of the sequencing and mapping.

Notably, the review and visualization tools disclosed herein include a plurality of different metrics that provide a user (e.g., a laboratory or medical practitioner) with a comprehensive suite of results in an accessible, streamlined format (e.g., sequencing validation, sequencing statistics, mapping validation, mapping statistics, microorganism detection, microbe-specific annotations, pathogen information, antimicrobial resistance (AMR) gene expression, and therapeutic treatments, among others). As discussed above, such features allow the analysis and interpretation of NGS ID data by users without advanced skills in each and every one of the various aspects of the analysis. In some embodiments, the provided review and visualization tools present a summary of the information relevant to analyzing the presence of microorganisms in a respective sample such that it can be efficiently examined, understood, and/or reviewed by a practitioner. Further customization is also possible for situations that necessitate fine-tuning.

In particular, in addition to an automated process for analysis of the presence of microorganisms, in some embodiments, any one of the parameters and/or detection thresholds can be adjusted based on user preference and/or a priori knowledge. In some such embodiments, the review and visualization tool can be modified to include an affordance for accepting one or more approvals (e.g., by a laboratory or medical technician, supervisor and/or director) prior to submission of the analysis of the results set for downstream processing. Each approval stage for a respective sample can be indicated by a review status. Furthermore, selection and/or approval at any stage of the approval process (e.g., first, second, third, and/or final approval) can be tagged with a user identity, an access timestamp, and/or a record of each change made in the respective sample. In some embodiments, final approval of a sample (e.g., a control and/or an analysis sample) removes the sample from the list of one or more pending samples.

In some embodiments, any one of the results in the results set can be separately approved or rejected, including the presence or absence of a detected microorganism, a passing score for a quality control metric, and/or a passing score for a sequencing or mapping statistic compared to a filtering threshold. Additional elements that can be customized include specific parameters or metrics to be presented on the display for each sample, batch, or run.

In some instances, further customization is also possible through an administrator access account, by controlling and managing filters, profiles, user accounts, groups, and/or permissions for specific users (e.g., granting review and/or approval access). For example, in some implementations, a production workflow can be established by restricting access to analysis samples until one or more control samples are finally approved. In some embodiments, specific filters or profiles can be established for specific scenarios, such as in instances where it is desirable to develop, optimize and validate a user-modified, custom set of parameters and detection thresholds that is subsequently applied, consistently, to all future samples in the workflow.

The systems and methods disclosed herein further include using the review and visualization tool to generate a report (e.g., a diagnostic report). In some embodiments, the report is generated as a printable document (e.g., a PDF). In some embodiments, the report is generated as an email that can be sent to, for example, a patient, a medical practitioner, and/or a clinical institution. As with the customization of the display, additional elements that can be customized include the specific parameters, metrics, and/or results to be included in the report (e.g., sequencing validation, sequencing statistics, mapping validation, mapping statistics, list of detected microorganisms, microbe-specific annotations, pathogen status, presence or absence of antimicrobial resistance (AMR) genes, antimicrobial resistance (AMR) gene annotations, and/or therapeutic treatments based on any of the above results or any combinations thereof).

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the term “microorganism,” or “microbe,” refers to a microscopic organism. In some embodiments, the term “microorganism” will be understood to include bacteria, fungi, protozoa (e.g., protozoan parasites), viruses (e.g., DNA viruses and/or RNA viruses), algae, archaea, phages, and/or helminths (e.g., multicellular eukaryotic parasites). In some embodiments, a microorganism is a single-celled organism and/or a colony of single-celled organisms. In some embodiments, a microorganism is eukaryotic or prokaryotic. In some embodiments, a microorganism is a pathogen (e.g., disease-causing), such as a human, animal, or plant-infective pathogen.

Examples of bacteria include, but are not limited to, disease-causing agents such as Acinetobacter baumanii, Actinobacillus sp., Actinomycetes, Actinomyce sp. (such as Actinmyces israeli and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae), Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis, and Brucella suis), Burkholderia sp. (such as Burkholderia pseudomallei and Burkholderia cepacia), Campylobacter sp. (such as (Campylobacter jejuni, Campylobacter coli, Camplobacter lari and (Campylobacter fetus), Capnocytophaga sp., Cardiobacterium hominis, Chlamydia trachomatis, Chlamydophila pneumoniae, Chylamydophilapsuttaci, Citrobacter sp. Coxiella burnetii, Corynebacterium sp. (such as, Corynebacterium diphtheriae, Corynebacterium jeikeium and Corynebacterium), Clostridium sp. (such as Clostridium perfringens, Clostridium dificile, Clostridium botulinum and Clostridium telani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli), Enterococcus sp. (such as Enterococcus faecalis and Enterococcus faecium), Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus), Helicobacter sp. (such as Helicobacter pylon, Helicobacter cinaedi and Heliobacter fennelliae), Kingella kingh, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia haemolytica, Microsporum canis, Moraxella catarrhalis, Morganella sp., Mobiluncus sp., Micrococcus sp., Myobacterium sp. (such as Mycobacterium leprae, Mycobacterium tuberculosis, Mycobacterium paratuberculosis, Myobacterium intracelluare, Mycobacterium avium, Mycobacterium bovis, and Mycobacterium marinum), Mycoplasma sp. (such as Mycoplasma pneumoniae, Mycoplasma homirus, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitis), Pasteurella multocida, Pityrosporum orbiculare (Malassezia furfur). Plesiomonas shigelloides, Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serrana sp (such as Serrana marcesans and Serratia liquefaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus haemolyticus, Staphylococcus saprophyticus), Streptococcus sp (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B1 streptococci, Streptococcus agalactiae, Group C streptococci, Streptococcus anginosus, Streptococcus equisimilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum), Trichophyton rubrum, T. mentagrophytes, Tropheryma whippehi, Ureaplasma urealyticum, Veillonella sp., Vibrio sp. (such as Vibrio cholerae, Vibro parahaemolyticus, Vibro vulnificus, Vibrio parahaemolyticus, Vibrio vulnificus, Vibrio alginolyticus, Vibrio mimicus, Vibrio hollisae, Vibrio fluvialis, Vibrio metchnikovii, Vibro damsela and Vibrio furnish), Yersinia sp. (such as Yersinia enterocolitica, Yersinia pestis, and Yersmia pseudotuberculosis) and Xanthomonas maltophilia.

Examples of fungi include, but are not limited to, Aspergillus sp., Candida auris, Candida albicans, Candida dubliniensis, Candida famata, Candida glabrata, Candida guilliermondii, Candida kefyr, Candida lusitaniae, Candida krusei, Candida parapsilosis, Candida tropicalis, Cryptococcus gatii, Cryptococcus neoformans, Fusarium sp., Malassezia furfur, Rhodotorula sp., Trichosporon sp., Histoplasma capsulatum, Coccidioides immitis, and Pneumocystis carinii, as well as the causative agents of Aspergillosis, Balsomycosis, Candidiasis, Coccidioidomycosis, fungal eye infections, fungal nail infections, histoplasmosis, mucormycosis, mycetoma, Pneuomcystis pneumonia, ringworm, sporotrichosis, crypococcosis, and Talaromycosis.

Examples of protozoan parasites include, but are not limited to, Plasmodium falciparum, P. vivax, P. ovals P. malariae, P. berghei, Leishmania donovani, L, infantum, L. chagasi, L. mexicana, L. amazonensis, L. venezuelensis, L. tropica, L. major, L. minor, L aethiopica, L. Biana braziliensis, L. (V.) guyanensis, L. (V.) panamensis, L. (V.) periviana, Trypanosoma brucei rhodesiense, T. brucei gambiense, T. cruzi, Giardia intestinalis, G. lamblia, Toxoplasma gondii, Entamoeba histolytica, Trichomonas vaginalis, Pneumocystis carnii, and Cryptosporidium parvum.

Examples of helminths include, but are not limited to, Filarioidea sp., Wuchereria sp. (such as Wuchereria bancrofti), Brugia sp. (such as Brugia malayi and Brugia timori), Loa sp. (such as Loa loa), Mansonella sp. (such as Mansonella streptocerca, Mansonella perstans, and Monsonella ozzardi), Onchocerca sp. (such as Onchocerca volvulus). Enterobius vermicularis, Ascaris sp. (such as Ascaris lumbricoides), Dracunculus (such as Dracunculus medinensis), Ancylostoma sp. (such as Ancylostoma duodenale, Ancylostoma braziliense, Ancylostoma tubaeforme, and Ancylostoma caninum), Necator sp. (such as Necator americanus), Trichuris sp. (such as Trichuris trichiura, Trichuris vulpis, Trichuris campanula, Trichuris suis, and Trichuris muris), Strongyloides sp. (such as Strongyloides stercoralis, Strongyloides canis, Strongyloides fuelleborni, Strongyloides cebus, and Strongyloides kellyi), Nematodirus sp., Moniezia sp., Oesophagostomum sp. (such as Oesophagostomum bifurcum, Oesophagostomum aculeatum, Oesophagostomum brumpti, Oesophagostomum stephanostomum, and Oesophagostomum stephanostomum var thomasi), Cooperia sp. (such as Cooperia ostertagi and Cooperia oncophora), Haemonchus sp., Ostertagia sp. (such as Ostertagia ostertagi), Trichostrongylus sp. (such as Trichostrongylus axei), Dirofilaria sp. (such as Dirofilaria immitis, Dirofilaria tenuis and Dirofilaria repens), and Schistosoma sp. (such as Schistosoma incognitum, Schistosoma ovuncatum, Schistosoma sinensium, Schistosoma indicum, Schistosoma nasale, Schistosoma spindale, Schistosoma japonicam, Schistosoma malayensis, Schistosoma mekongi, Schistosoma haematobium, Schistosoma bovis, Schistosoma curassoni, Schistosoma guineensis, Schistosoma haematobium, Schistosoma intercalatum, Schistosoma leiperi, Schistosoma margrebowiei, Schistosoma mattheei, Schistosoma mansoni, Schistosoma edwardiense, Schistosoma hippotami, and Schistosoma rodhaini)

Examples of viruses include, but are not limited to, disease-causing agents such as Adeno-associated virus, Aichi virus, Australian bat lyssavirus, BK polyomavirus, Banna virus, Barmah forest virus, Bunyamwera virus. Bunyavirus La Crosse, Bunyavirus snowshoe hare, Cercopithecine herpesvirus, Chandipura virus, Chikungunya virus. Coronavirus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus. European bat lyssavirus. GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus. Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1. Human papillomavirus 2. Human papillomavirus 16,18, Human parainfluenza, Human parvovirus B19, Human respiratory syncytial virus. Human rhinovirus, Human SARS coronavirus. Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus. Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus. Murray valley encephalitis virus, New York virus, Nipah virus, Norwalk virus, Norovirus. O'nyong-nyong virus, Orf virus, Oropouche virus, Pichinde virus, Poliovirus. Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A. Sandfly fever sicilian virus, Sapporo virus, Semliki forest virus, Seoul virus, Severe acute respiratory syndrome coronavirus 2, Simian foamy virus, Simian virus 5. Sindbis virus, Southampton virus, St. louis encephalitis virus. Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, the term “microorganism” will be understood to include any one or more bacteria, fungi, protozoa, viruses, algae, archaea, phages, and/or helminths selected from a database (e.g., a microbial genome database, a transcriptomic database, a proteomic database, a metabolomics database, a taxonomic database, and/or a clinical database). In some embodiments, the database comprises one or more entries corresponding to and/or identifying a microorganism (e.g., an annotation, for a respective microorganism, to a genome, transcriptome, nucleic acid sequence, protein sequence, metabolite, taxonomic record and/or clinical record). In some embodiments, a microorganism is selected from a database that is locally maintained, proprietary, and/or open-access. In some embodiments, a microorganism is selected from a national and/or international database. Examples of such databases include, but are not limited to, NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. For example, MBGD comprises all complete genome sequences of bacteria, archaea, and unicellular eukaryotes, including fungi and protozoa, available at the NCBI genomes site. The Microbial Rosetta Stone is a database that provides information on disease-causing organisms (e.g., bacteria, fungi, protozoa, DNA viruses, RNA viruses, plants, and animals) and the toxins produced therefrom. See, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1). D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

As used herein, the terms “antimicrobial resistance marker” or “AMR marker” refers to a measurable and/or detectable marker indicating that a respective microorganism has antimicrobial resistance. As used herein, the term “antimicrobial resistance” refers to a property of or exhibited by a respective microorganism, such that the respective microorganism is resistant to one or more antimicrobial interventions (e.g., where an effect of an antimicrobial intervention is attenuated, obstructed, or negated). As used herein, the term “antimicrobial susceptibility” refers to a property of or exhibited by a respective microorganism, such that the respective microorganism is susceptible to one or more antimicrobial interventions (e.g., where an effect of an antimicrobial intervention serves to kill, diminish, slow or prevent growth in one or a population of microorganisms).

In some embodiments, antimicrobial resistance is conferred by a genetic sequence (e.g., an antimicrobial resistance gene). In some embodiments, the antimicrobial resistance marker is a genetic marker (e.g., a nucleic acid sequence for the antimicrobial resistance gene indicating that the gene comprises a mutation that confers resistance). In some embodiments, the antimicrobial resistance marker is a restriction fragment length polymorphism (RFLP), a random amplified polymorphic DNA (RAPD), an amplified fragment length polymorphism (AFLP), a variable number tandem repeat (VNTR), an oligonucleotide polymorphism (OP), a single nucleotide polymorphism (SNP), an allele specific associated primer (ASAP), an inverse sequence-tagged repeat (ISTR), an inter-retrotransposon amplified polymorphism (IRAP), and/or a simple sequence repeat (SSR or microsatellite). In some embodiments, an antimicrobial resistance marker is detected based on a mapping (e.g., an alignment) of one or more nucleotide sequences to a reference sequence (e.g., a reference genome). In some embodiments, an antimicrobial resistance marker is an amino acid sequence and/or an amino acid residue. In some embodiments, an antimicrobial resistance marker is a biochemical marker.

In some embodiments, an antimicrobial resistance marker indicates that a respective microorganism is resistant to one or more interventions for a corresponding type of microorganism (e.g., antibacterial resistance, antiprotozoal resistance, antifungal resistance, antihelminthic resistance, and/or antiviral resistance). For example, in some embodiments, an antimicrobial intervention is a drug that targets a specific gene in a respective microorganism, and a mutation in the gene confers resistance to the microorganism. In some such embodiments, an antimicrobial resistance marker can be a genetic marker for the target gene that indicates a resistance to the antimicrobial drug.

As used herein, the term “antimicrobial resistance status” refers to an indication of a presence or absence of an antimicrobial resistance marker. For example, the term antimicrobial resistance status or AMR status will be understood to include an indication that a respective biological and/or non-biological sample and/or a microorganism detected in a sample has either antimicrobial resistance or antimicrobial susceptibility. In some embodiments, an antimicrobial resistance status includes an indication that an antimicrobial resistance marker is present (e.g., has been detected) in the respective sample and/or microorganism. In some embodiments, an antimicrobial resistance status includes an indication of any one or more features for the respective antimicrobial resistance marker (e.g., gene identifier, gene name, intervention (drug) information, intervention (drug) classes, associated organisms, gene families, and/or resistance mechanisms).

In some embodiments, an antimicrobial resistance marker is associated with one or more microorganisms in a plurality of microorganisms (e.g., where the respective microorganism has been reported or annotated as expressing the respective antimicrobial resistance marker). In some embodiments, a first antimicrobial resistance marker is associated with a first respective microorganism in a plurality of microorganisms, and a second antimicrobial resistance marker is associated with a second respective microorganism, other than the first microorganism, in the plurality of microorganisms.

Examples of antimicrobial resistance markers (e.g., genes and/or amino acid residues) include, but are not limited to, the antimicrobial resistance markers listed below in Table 1.

TABLE 1
Example Antimicrobial Resistance Markers

| Intervention Type | Marker: Gene Name or Subtype [AA Mutation] |
|---|---|
| Antibiotic | Aminocoumarins: GyrB, ParE, ParY |
| Resistance | Aminoglycosides: AAC(1), AAC(2′), AAC(3), AAC(6′), ANT(2″), |
| | ANT(3″), ANT(4″), ANT(6), ANT(9), APH(2″), APH(3″), APH(3′), |
| | APH(4), APH(6), APH(7″), APH(9), ArmA, RmtA, RmtB, RmtC, |
| | Sgm |
| | β-Lactams: AER, BLA1, CTX-M, KPC, SHV, TEM; BlaB, CcrA, |
| | IMP, NDM, VIM; ACT, AmpC, CMY, LAT, PDC, OXA β- |
| | lactamase; methicillin-resistant PBP2; antibiotic-resistant Omp36, |
| | OmpF, PIB (por); bla (blaI, blaR1) and mec (mecI, mecR1) operons |
| | Chloramphenicol: CAT; Chloramphenicol phosphotransferase |
| | Ethambutol: EmbB |
| | Mupirocin: MupA, MupB |
| | Peptide antibiotics: MprF |
| | Phenicol: Cfr 23S rRNA methyltransferase |
| | Rifampin: Arr; Rifampin glycosyltransferase; Rifampin |
| | monooxygenase; Rifampin phosphotransferase; DnaA, RbpA; RpoB |
| | Streptogramins: Cfr 23S rRNA methyltransferase; ErmA, ErmB, |
| | Erm(31); Lsa, MsrA, Vga, VgaB; Streptogramin Vgb lyase; Vat |
| | acetyltransferase |
| | Fluoroquinolones: Fluoroquinolone acetyltransferase; |
| | Fluoroquinolone-resistant GyrA, GyrB, ParC, Qnr |
| | Fosfomycin: FomA, FomB, FosC; FosA, FosB, FosX |
| | Glycopeptides: VanA, VanB, VanD, VanR, VanS |
| | Lincosamides: Cfr 23S rRNA methyltransferase; ErmA, ErmB, |
| | Erm(31); Lin |
| | Linezolid: Cfr 23S rRNA methyltransferase |
| | Macrolides: Cfr 23S rRNA methyltransferase; ErmA, ErmB, |
| | Erm(31); EreA, EreB; GimA, Mgt, Ole; MPH(2′)-I, MPH(2′)-II; |
| | MefA, MefE, Mel |
| | Streptothricin: sat |
| | Sulfonamides: Sul1, Sul2, Sul3, sulfonamide-resistant FolP |
| | Tetracyclines: Mutant porin PIB (por) with reduced permeability; |
| | TetX; TetA, TetB, TetC, Tet30, Tet31; TetM, TetO, TetQ, Tet32, |
| | Tet36 |
| | Antibiotic efflux: MacAB-TolC, MsbA, MsrA, VgaB; EmrD, |
| | EmrAB-TolC, NorB, GepA; MepA; AdeABC, AcrD, MexAB- |
| | OprM, mtrCDE, |
| | EmrE; adeR, acrR, baeSR, mexR, phoPQ, mtrR |
| Antifungal | CYP51a [F219S, F46Y, M172V, N248T, D255E, G138C, G138S, |
| Resistance | G434C, G54E, I266N, G54R, G54V, G54W, H147Y, L98H, M217I, |
| | M220L, M220T, M220V, P216L, R228Q, Y121F, T289A, G448S, |
| | M172I, Y431C] |
| | ERG11 [A114S, G487T, T916C, A61V, D116E, D225H, D225Y, |
| | E165K, E266D, F126L, F126T, F145L, F380S, F449L, F449Y, |
| | F72L, G129A, G307S, G448V, G450E, G464S, G484S, H283R, |
| | I253V, I471T, K119L, K119N, K128T, R467I, K143E, K143Q, |
| | K143R, K161N, L491V, M140R, P375Q, P49R, T486P, P503L, |
| | Q474K. R163T. R381I, R467K, S405F, T132H, T229A, T494A, |
| | V437I, V452A. V488I. V130I, Y132F, Y132H, Y136F, Y205E, |
| | G472R, Y257H, Y33C. Y39C. Y79C, T199I] |
| | tub2 [E198A, H6Y] |
| | FKS1 [D632E, D632G, D632Y, D646Y, F639I, F641S, F655C, |
| | L642S, N470K, P660A, S639F, S639P, S645F, S645P, S645Y, |
| | V641K] |
| | CYP51b [G460S, S508T] |
| | CYP51c [Y319H, T788G] |
| | MgCYP51 [L50S, V136A, Y461S, S524T, Y459C, Y459S, G460D] |
| | MfCYP51 [A313G, Y463H, Y136F, Y463D, Y461D, Y463N] |
| | FUR1 [R101C, F211I] |
| | FKS2 [F659del, F659S, F659V] |
| | BcSdhB [P225F, H272Y, H272R] |
| | CYP51 [A29P, D78Y, E106K, E331A, F506I, G459S, G511S, |
| | I381V, I440V, K23E, K449R, K508R, M144T, N244S, Q167H, |
| | Q309H, Q43H, R462H, S35T, S505Q, S507P, V37A, V55A, Y133F, |
| | Y134F, Y136F, Y136H, Y137H, Y486H] |
| | DHPS [T55A, P57S] |
| | Cytb [G143A] |
| | RTA2 [G234S] |
| | HapE [P88L] |
| | cox10 [R243Q] |
| | DHFR [D153V, S37T, I158V, V79I, Y197L, T14A, P26Q, M52I, |
| | E63G, T144A, KI71E, S106P, E127G, R170G] |
| Antiprotozoal | Pfmdr1 [N86Y, Y184F, S1034C, N1042D, 1246Y] |
| Resistance | Pfcrt [K76T, C72S, M74I, N75E, A220S, Q271E, N326S, I356T, |
| | R371I] |
| | Pfmrp [Y191H, A437S] |
| | Pfnhe1 [ms4670] |
| | PfATP4 [G223R] |
| | Pfdhps [S436A/F, A437G, K540E, A581G, A613T/S, A16V, N51I, |
| | C59R, I164L] |
| | PfAtp18 [T38I] |
| | PfK13 [Y493H, R539T, I543T, C580Y, M476I, D56V, F446I, |
| | P574L] |
| | Pfcytb [Y268S/C/N] |
| | MRP1, HSP70, PRP1 (Leishmania) |
| | LdMT [L856P, T420N, L832F, V176D, W210, Y354F, F1078Y] |
| | LdRos3 [M1] |
| Antihelminthic | beta-tubulin [F200Y, E198A. F167Y] |
| Resistance | unc-38 |
| | unc-63 |
| | acr-8 |
| | mptl-1 |
| | des-2 |
| | deg-3 |
| | avr-14 [L256F] |
| | lgc-37 [K169R] |
| | glc-5 [A169 V] |
| | ggr-3 |
| | pgpA |
| Antiviral | A H1N1 [H275Y, Q136K, N70S, I222V/M, Y155H] |
| Resistance | A H1N1 pdm09 [N294S, H275Y, I222V, I222R, E119G, E119V, |
| | N325K, S247N, I117V] |
| | A H3N2 [R292K, N294S, D151A/E, Q136K, E119V/A/D/G, |
| | R224K, R371K, R224K, E276D, H274Y, I222V] |
| | B [E119A/D/G/A, H274Y, R371K, I222T, R292K, N294S, D198N, |
| | D198E] |

See, for example, Capela et al., 2019, “An Overview of Drug Resistance in Protozoal Diseases,” Int J Mol Sci. 20(22): 5748; doi: 10.3390/ijms20225748; Beech et al., 2011, “Anthelmintic resistance: markers for resistance, or susceptibility?” Parasitology 138(2): 160-174; doi: 10.1017/S0031182010001198; and Toledu-Rueda et al., 2018, “Antiviral resistance markers in influenza virus sequences in Mexico, 2000-2017,” Infect Drug Resist 11: 1751-1756; doi: 10.2147/IDR.S153154; each of which is hereby incorporated herein by reference in its entirety.

In some embodiments, the term “antimicrobial resistance marker” will be understood to include any one or more genes, amino acid sequences amino acid residues, genetic markers, and/or biochemical markers selected from a database. In some embodiments, an antimicrobial resistance marker is selected from a database that is one or more of locally maintained, proprietary, and/or open-access. In some embodiments, an antimicrobial resistance marker is selected from a national and/or international database. Examples of such databases include, but are not limited to, the National Database of Antibiotic Resistant Organisms (NDARO), the Comprehensive Antibiotic Resistance Database (CARD), ResFinder, PointFinder, ARG-ANNOT, ARGs-OSP, PlasmoDB, the Mycology Antifungal Resistance Database (MARDy), DBDiaSNP, the HIV Drug Resistance Database, the Virus Pathogen Resource (ViPR), and/or any of the databases used for selecting one or more microorganisms, as disclosed above. See, for example, McArthur et al., 2013, “The Comprehensive Antibiotic Resistance Database,” Antimicrob Ag Chemother, 57(7) 3348-3357; doi: 10.1128/AAC.00419-13; Zankari et al., 2017, “PointFinder: a novel web tool for WGS-based detection of antimicrobial resistance associated with chromosomal point mutations in bacterial pathogens,” J Antimicrob Chemother. 72 (10) 2764-2768: doi: 10.1093/jac/dkx217; Gupta et al., 2013, “ARG-ANNOT, a New Bioinformatic Tool To Discover Antibiotic Resistance Genes in Bacterial Genomes,” Antimicrob Ag Chemother, 58 (1) 212-220; doi: 10.1128/AAC.01310-13; Zhang et al., “ARGs-OSP: online searching platform for antibiotic resistance genes distribution in metagenomic database and bacterial whole genome database,” bioRxiv 337675; doi: 10.1101/337675; Nash et al., 2018, “MARDy: Mycology Antifungal Resistance Database,” 34 (18) 3233-3234; doi: 10.1093/bioinformatics/bty321; and Mehla and Ramana, 2015, “DBDiaSNP: An Open-Source Knowledgebase of Genetic Polymorphisms and Resistance Genes Related to Diarrheal Pathogens,” OMICS 19 (6) 354-360: doi: 10.1089/omi.2015.0030; each of which is hereby incorporated herein by reference in its entirety.

As used herein, the terms “sample,” “biological sample,” “patient sample.” or “analysis sample” refers to any sample taken from a biological or non-biological subject and/or source, which can reflect a biological or non-biological state associated with the subject and/or source. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A sample can be a liquid sample or a solid sample (e.g., a cell or tissue sample). A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample). Examples of non-biological samples include, but are not limited to, agricultural samples, environmental samples, laboratory samples, water samples (e.g., from an external, internal, natural, and/or man-made water source), air samples, terrestrial samples, and/or extraterrestrial samples. Non-biological samples can be solid, liquid, and/or gaseous. For example, a non-biological sample can include a frozen sample. Non-biological samples can include by-products (e.g., of industrial, chemical, agricultural, laboratory, and/or food processes). Any other non-biological samples are contemplated, as will be apparent to one skilled in the art.

As used herein, the terms “nucleic acid” and “nucleic acid molecule” are used interchangeably. The terms refer to nucleic acids of any composition form, such as ribonucleic acid (RNA), deoxyribonucleic acid (DNA, e.g., complementary DNA (cDNA), genomic DNA (gDNA) and the like), and/or DNA or RNA analogs (e.g., containing base analogs, sugar analogs and/or a non-native backbone and the like). In some embodiments, nucleic acids are in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid, in some embodiments, can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As used herein, the terms “sequencing,” “sequencing reaction,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript, a DNA fragment and/or a genomic locus.

As used herein, the term “NGS ID” refers to the use of enrichment-independent and/or enrichment-based sequencing (e.g., next-generation sequencing (NGS)), to detect, measure, and/or profile one or more nucleic acid molecules obtained from one or more microorganisms and/or hosts. In some embodiments, the nucleic acids correspond to markers (e.g., AMR markers). In some embodiments, NGS ID further includes determining the role of microbial and host markers on health, infectious diseases, and/or other diseases.

As used herein, the term “nucleotide sequences,” “sequence reads,” “sequencing reads,” or “reads” refers to nucleotide base sequences produced by any nucleic acid sequencing process described herein or known in the art. Nucleotide sequences can be generated from one end of nucleic acid fragments (e.g., “single-end reads”) or from both ends of nucleic acid fragments (e.g., paired-end reads, double-end reads). The length of the nucleotide sequence is often associated with the particular sequencing technology. High-throughput methods, for example, provide nucleotide sequences that can vary in size from tens to hundreds of base pairs (bp). In some embodiments, the nucleotide sequences are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the nucleotide sequences are of a mean, median or average length of about 1000 bp, 2000 bp, 5000 bp, 10,000 bp, or 50,000 bp or more. Nanopore® sequencing, for example, can provide nucleotide sequences that can vary in size from tens to hundreds to thousands of base pairs. Illumina® parallel sequencing, for example, can provide nucleotide sequences that do not vary as much, where, for example, most of the nucleotide sequences can be smaller than 200 bp. A nucleotide sequence can refer to sequence information corresponding to a nucleic acid molecule (e.g., a string of nucleotides). For example, a nucleotide sequence can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A nucleotide sequence can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “nucleotide sequence count,” “sequence read count,” or “read count” refers to the total number of nucleic acid reads generated, which may or may not be equivalent to the number of nucleic acid molecules generated, during a nucleic acid sequencing reaction. In some embodiments, a nucleotide sequence count refers to a count of nucleotide sequences in the plurality of nucleotide sequences that map (e.g., align) to a corresponding reference sequence (e.g., complete and/or incomplete genome) for a respective microorganism. In some embodiments, a nucleotide sequence count refers to a count of unique nucleotide sequences in the plurality of nucleotide sequences that map to a corresponding reference sequence (e.g., complete and/or incomplete genome) for a respective microorganism. In some embodiments, a nucleotide sequence count refers to a count of nucleotide sequences in the plurality of nucleotide sequences that satisfy a criterion, such as a pre-processing criterion, a mapping statistic threshold (e.g., an alignment identity threshold), and/or a sequencing statistic threshold.

As used herein, the term “depth,” “read depth,” or “sequencing depth” refers to a total number of unique nucleic acid fragments encompassing a particular locus or region of the reference sequence (e.g., complete and/or incomplete genome) of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is an integer, because it represents the actual sequencing depth for a particular locus. Sequencing depth can also be applied to multiple loci, or a whole genome or reference sequence, in which case Y can refer to the mean or average number of times a locus or a haploid genome, or a whole genome or reference sequence, respectively, is sequenced. Alternatively, depth, read-depth, or sequencing depth can refer to a measure of central tendency (e.g., a mean or mode) of the number of unique nucleic acid fragments that encompass one of a plurality of loci or regions of the genome or reference sequence of a subject that are sequenced in a particular sequencing reaction. For example, in some embodiments, sequencing depth refers to the average depth of every locus across an arm of a chromosome, a targeted sequencing panel, an exome, or an entire genome or reference sequence. In such case, Y may be expressed as a fraction or a decimal, because it refers to an average depth across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “coverage” refers to the proportion of a reference sequence (e.g., a complete and/or incomplete reference genome) that is covered by mapped (e.g., aligned) nucleotide sequences. In some embodiments, coverage is a percent coverage of the mapping of a plurality of nucleotide sequences against the respective reference sequence. For instance, in some embodiments, if after mapping of a plurality of nucleotide sequences to a reference sequence, 90% of the reference sequence is covered by mapped (e.g., aligned) reads, then the coverage is 90%.

As used herein, the terms “genome” or “reference genome” refer to any particular known, sequenced or characterized genome, whether partial or complete, of any organism or virus that may be used to reference identified sequences from a subject. Example reference genomes used for human subjects as well as many other organisms are provided in the online genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome is an assembled or partially assembled genomic sequence from one or more microorganisms of the same species. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

In some embodiments, a genome is a complete genome. In some embodiments, a genome is an incomplete genome. For example, in some embodiments, an incomplete genome is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the complete genome.

In some embodiments, a complete or incomplete genome is less than 1 megabase pairs (Mb), less than 0.5 Mb, less than 0.4 Mb, less than 0.3 Mb, less than 0.2 Mb, or less than 0.1 Mb. In some embodiments, a complete or incomplete genome is at least 1 Mb, at least 2 Mb, at least 3 Mb, at least 4 Mb, at least 5 Mb, at least 6 Mb, at least 7 Mb, at least 8 Mb, at least 9 Mb, at least 10 Mb, at least 15 Mb, at least 20 Mb, at least 25 Mb, at least 30 Mb, at least 35 Mb, at least 40 Mb, at least 45 Mb, at least 50 Mb, at least 100 Mb, at least 200 Mb, at least 500 Mb, at least 1,000 Mb, at least 2,000 Mb, at least 3,000 Mb, at least 4,000 Mb, at least 5,000 Mb, at least 10 gigabase pairs (Gb), at least 20 Gb, or at least 50 Gb.

In some embodiments, a complete or incomplete genome spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, at least 2,000, at least 3,000, at least 4,000, at least 5,000, at least 10,000, or at least 50,000 genes. In some embodiments, a complete or incomplete genome spans a region of a reference genome comprising between 1 and 10, between 10 and 50, between 50 and 100, between 100 and 500, between 500 and 1000, between 1000 and 2000, between 2000 and 5000, between 5000 and 10,000, between 10,000 and 50,000, or more than 50,000 genes.

In some embodiments, a complete or incomplete genome spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, or at least 500 antimicrobial resistance markers. In some embodiments, a complete or incomplete genome spans a region of a reference genome comprising between 1 and 10, between 10 and 50, between 50 and 100, or more than 100 antimicrobial resistance markers.

In some embodiments, a complete or incomplete genome is obtained from one or more nucleotide sequence databases and/or microorganism databases, including but not limited to NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. See, for example, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19: each of which is hereby incorporated by reference herein in its entirety.

As used herein, the term “reference sequence” refers to a sequence of nucleotide bases. In some embodiments, a reference sequence is a reference genome. In some embodiments, a reference sequence is a complete or incomplete genome. In some embodiments, a reference sequence is less than 1 megabase pairs (Mb), less than 0.5 Mb, less than 0.4 Mb, less than 0.3 Mb, less than 0.2 Mb, or less than 0.1 Mb in length. In some embodiments, a reference sequence is at least 1 Mb, at least 2 Mb, at least 3 Mb, at least 4 Mb, at least 5 Mb, at least 6 Mb, at least 7 Mb, at least 8 Mb, at least 9 Mb, at least 10 Mb, at least 15 Mb, at least 20 Mb, at least 25 Mb, at least 30 Mb, at least 35 Mb, at least 40 Mb, at least 45 Mb, at least 50 Mb, at least 100 Mb, at least 200 Mb, at least 500 Mb, at least 1,000 Mb, at least 2,000 Mb, at least 3,000 Mb, at least 4,000 Mb, at least 5,000 Mb, at least 10 gigabase pairs (Gb), at least 20 Gb, or at least 50 Gb in length.

In some embodiments, a reference sequence spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, at least 2,000, at least 3,000, at least 4,000, at least 5,000, at least 10,000, or at least 50,000 genes. In some embodiments, a reference sequence spans a region of a reference genome comprising between 1 and 10, between 10 and 50, between 50 and 100, between 100 and 500, between 500 and 1000, between 1000 and 2000, between 2000 and 5000, between 5000 and 10,000, between 10,000 and 50,000, or more than 50,000 genes.

In some embodiments, a reference sequence comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, or at least 500 antimicrobial resistance markers. In some embodiments, a reference sequence comprises between 1 and 10, between 10 and 50, between 50 and 100, or more than 100 antimicrobial resistance markers.

The implementations described herein provide various technical solutions for analysis of the presence of microorganisms in a result set obtained from a sequencing reaction of nucleic acids from a biological or non-biological sample. An example of such result sets are result sets arising from sample processing, sequencing, taxonomic classification and/or information presentation pipelines as disclosed in U.S. Patent Application No. 62/696,783, entitled “Methods and Systems for Processing Samples,” filed Jul. 11, 2018, PCT Application No. PCT/US2019/060915, entitled “Directional Targeted Sequencing,” filed Nov. 12, 2019, U.S. patent application Ser. No. 15/724,476, entitled “Methods and Systems for Multiple Taxonomic Classification,” filed Oct. 4, 2017, and U.S. Patent Application No. 62/723,384, entitled “Methods and Systems for Providing Sample Information,” filed Aug. 27, 2018, each of which is hereby incorporated by reference. Details of implementations are now described in conjunction with the Figures.

As used herein the term “k-mer” refers to a subsequence of a given length k within a longer sequence, where k is a positive integer of 2 or greater. In some embodiments, k is between three and one hundred. In some embodiments, k is between four and fifty. In some embodiments, k is between five and forty. In one example, the sequence “AGCTCT” is divided into the 3-nucleotide subsequences “AGC.” “GCT,” “CTC,” and “TCT.” In this example, each of these subsequences is a k-mer, where k=3. K-mers may be overlapping or non-overlapping. In some embodiments, k-mers overlap each other by one residue. K-mers and their use in sequence alignment and mapping are further described in Stokes and Glick, 2006, “MICA: desktop software for comprehensive searching of DNA databases,” BMC Bioinformatics 7:427; Kalafus, 2004, “Pash: Efficient Genome-Scale Sequence Anchoring by Positional Hashing,” Genome Research 14:672-678: and Mann and Noble, “Efficient identification of DNA hybridization partners in a sequence database,” Bioinformatics 14(22), e350-e358, each of which is hereby incorporated by reference.

### Example System Embodiments

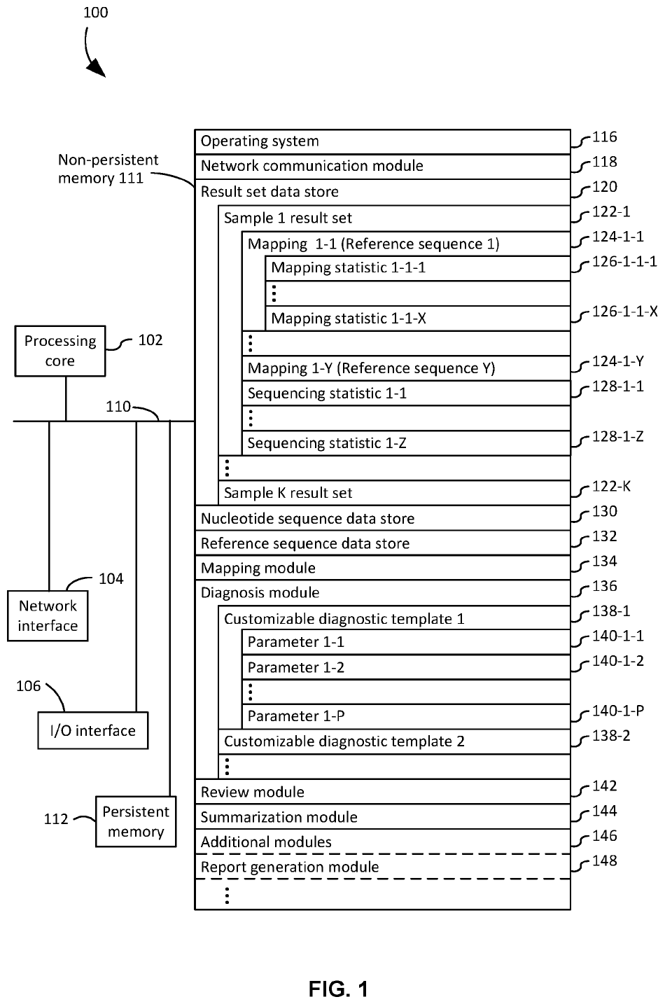

FIG. 1 is a block diagram illustrating a visualization system 100 in accordance with some implementations. The device 100 in some implementations includes one or more central processing units (CPU(s)) 102 (also referred to as processors), one or more network interfaces 104, a user interface 106, a non-persistent memory 111, a persistent memory 112, and one or more communication buses 110 for interconnecting these components. The one or more communication buses 110 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprises non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the visualization system 100 with other devices, or a communication network;
a result set data store 120 comprising a result set 122 (e.g., 122-1, . . . 122-K) obtained from a sequencing reaction of nucleic acids from a biological or non-biological sample, where the result set includes a plurality of sequencing statistics 128 (e.g., 128-1-1, . . . 128-1-Z) and mappings 124 of a plurality of nucleotide sequences against the reference sequences of a set of microorganisms (e.g., 124-1-1, . . . 124-1-Y for each mapping of the plurality of nucleotide sequences against each of Y reference sequences), and, for each mapping 124, a plurality of mapping statistics 126 (e.g., 126-1-1-1, . . . 126-1-1-X);
a nucleotide sequence data store 130 comprising a plurality of nucleotide sequences obtained from the sequencing reaction of nucleic acids from the biological or non-biological sample;
a reference sequence data store 132 comprising a plurality of reference sequences (e.g., complete and/or incomplete genomes) of a set of microorganisms;
a mapping module 134 for mapping the plurality of nucleotide sequences against the plurality of reference sequences of the set of microorganisms;
a diagnosis module 136 comprising a first customizable diagnostic template 138-1 and a second customizable diagnostic template 138-2, where the first customizable diagnostic template is applied to the result set and comprises a plurality of parameters 140 (e.g., 140-1-1, 140-1-2, . . . 140-1-P) for specifying a subset of the plurality of sequencing statistics 128, a subset of the set of microorganisms 132, and a subset of the plurality of mapping statistics 126, and where the second customizable diagnostic template is optionally applied to the result set;
a review module 142 including a review status for the nucleic acid sequencing data and an affordance for updating the review status for the nucleic acid sequencing data;
a summarization module 144 that generates a summary of the subset of the plurality of sequencing statistics and a summary of the subset of the plurality of mapping statistics for each respective microorganism in the subset of the set of microorganisms that satisfies a criterion defined by the plurality of parameters 140, responsive to the application of the first customizable diagnostic template to the result set;
additional modules 146; and
an optional report generation module for reporting data from the result set 122, the diagnosis module 136, the review module 142, the summarization module 144, and/or the additional modules 146.

In some embodiments, the plurality of parameters 140 in the first customizable diagnostic template includes a minimum mapping threshold for the mapping of the plurality of nucleotide sequences to the reference sequence (e.g., genome), for each respective microorganism in the set of microorganisms. In some embodiments, the review module and/or the summarization module is customizable via a customizable user interface. In some such embodiments, the customizable user interface comprises a customizable microorganism detection quantification construct, a customizable detection threshold filter, and/or a customizable quality control filter, among others.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data may be combined or otherwise re-arranged in various implementations. In some implementations, the non-persistent memory Ill optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “visualization system 100,” the figures are intended more as a functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

### Specific Embodiments of the Disclosure

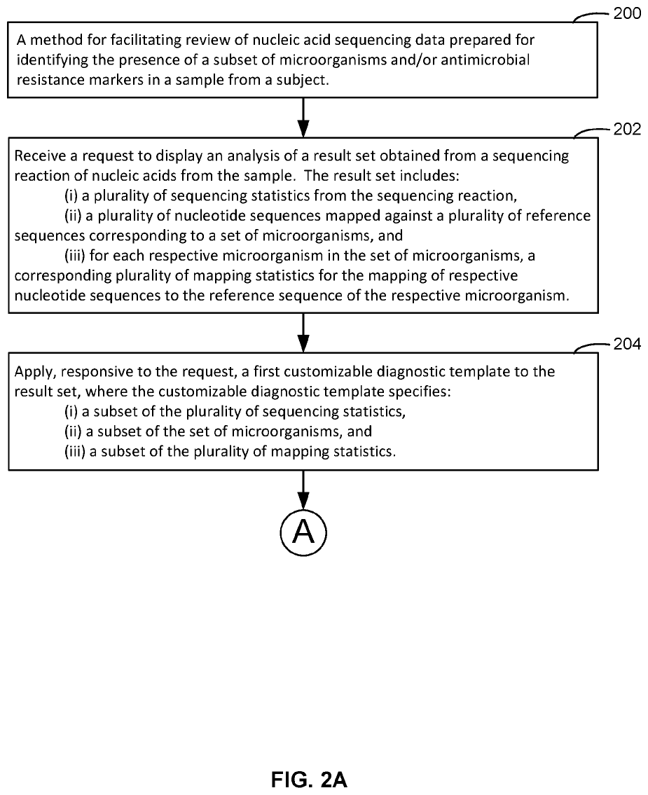

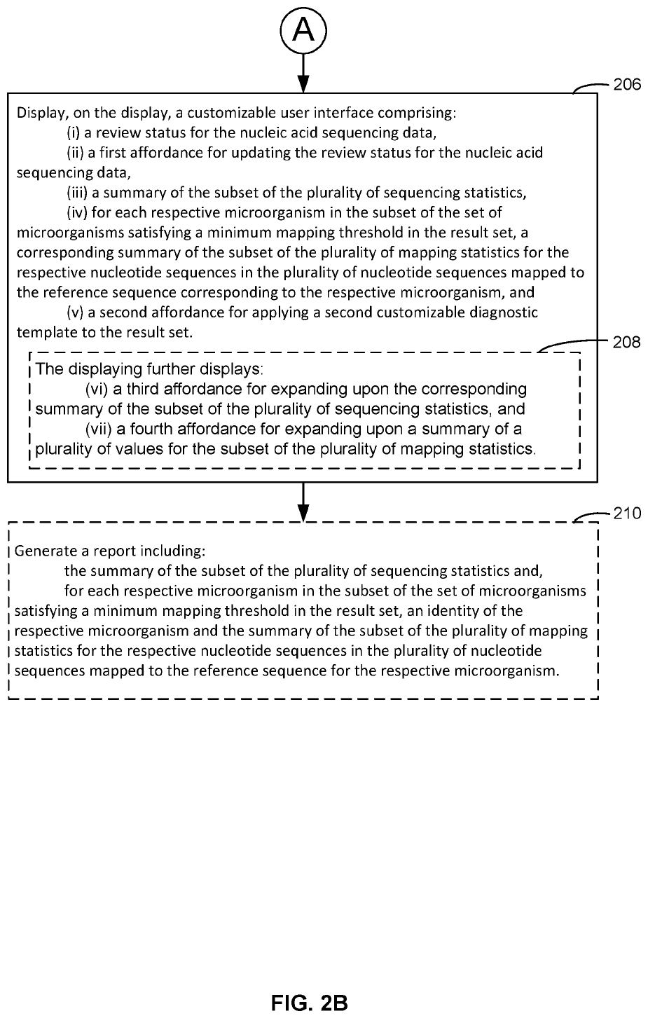

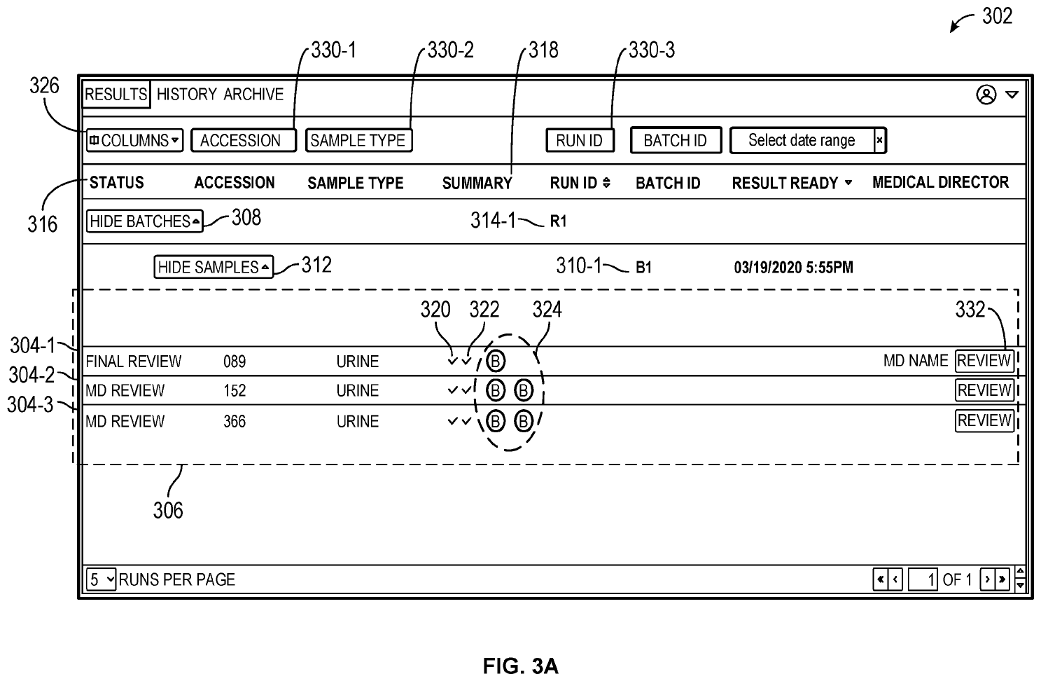

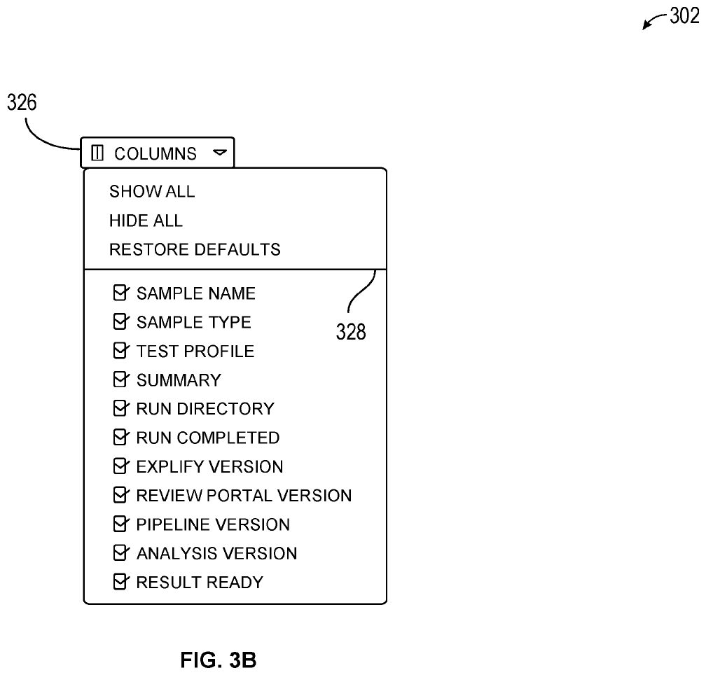

While a system in accordance with the present disclosure has been disclosed with reference to FIG. 1, a method in accordance with the present disclosure is now detailed with reference to FIGS. 2A-B and FIGS. 3-28. In some embodiments, the presently disclosed systems and methods are used in conjunction with the systems and methods described in, for example, IDbyDNA, 2019, “Explify Software v1.5.0 User Manual,” Document No. TH-2019-200-006, pp. 1-44, which is hereby incorporated by reference herein in its entirety for all purposes.

Referring to Block 200, the present disclosure provides a method for facilitating review of nucleic acid sequencing data 130 prepared for identifying the presence of a subset of microorganisms and/or antimicrobial resistance markers in a biological or non-biological sample (e.g., from a subject), at a computer system having a display, one or more processors, and memory storing one or more programs for execution by the one or more processors.

In an example embodiment, the present disclosure provides a review and visualization tool (e.g., comprising a display) for generating, viewing, modifying, validating, and/or reporting the results of a sequencing and mapping analysis using nucleic acids in a biological or non-biological sample obtained (e.g., from a subject such as a patient).

Subjects and Samples.

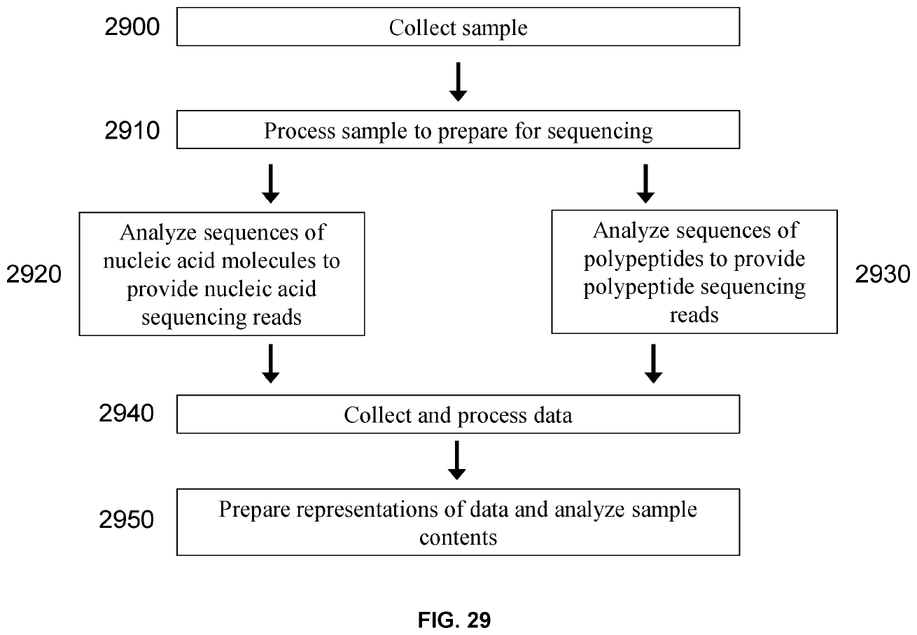

In some embodiments, a biological or non-biological sample (e.g., sample 304) is collected, prepared, sequenced (e.g., by next-generation sequencing), and mapped (e.g., aligned) to one or more reference sequences (e.g., complete and/or incomplete genomes) prior to the analysis of the presence of microorganisms. In some embodiments, sample processing is performed using any of the methods as disclosed in U.S. Patent Application No. 62/696,783, entitled “Methods and Systems for Processing Samples,” filed Jul. 11, 2018, which is hereby incorporated by reference herein in its entirety. In some embodiments, sample processing is performed using the method described in Example 2 and FIG. 29 (see Examples, below).

In some embodiments, the biological or non-biological sample is obtained from a subject (e.g., a biological subject). For example, in some embodiments, the subject is a human (e.g., a patient). In some embodiments, the biological or non-biological sample is obtained from any tissue, organ or fluid from the subject (e.g., urine sample 304-1). In some embodiments, a plurality of biological or non-biological samples is obtained from the subject (e.g., a plurality of replicates and/or a plurality of samples including a healthy sample and a diseased sample). In some embodiments, the biological or non-biological sample is obtained from a human with a disease condition. In some embodiments, the disease condition is influenza, common cold, measles, rubella, chickenpox, norovirus, polio, infectious mononucleosis (mono), herpes simplex virus (HSV), human papillomavirus (HPV), human immunodeficiency virus (HIV), viral hepatitis (e.g., hepatitis A, B, C. D. and/or E), viral meningitis, West Nile Virus, rabies, Ebola, strep throat, bacterial urinary tract infections (UTIs) (e.g., coliform bacteria), bacterial food poisoning (e.g., E. coli, Salmonella, and/or Shigella), bacterial cellulitis (e.g., Staphylococcus aureus (MRSA)), bacterial vaginosis, gonorrhea, chlamydia, syphilis. Clostridium difficile (C. diff), tuberculosis, whooping cough, pneumococcal pneumonia, bacterial meningitis, Lyme disease, cholera, botulism, tetanus, anthrax, vaginal yeast infection, ringworm, athlete's foot, thrush, aspergillosis, histoplasmosis, Cryptococcus infection, fungal meningitis, malaria, toxoplasmosis, trichomoniasis, giardiasis, tapeworm infection, roundworm infection, pubic and head lice, scabies, leishmaniasis, and/or river blindness.

In some embodiments, the biological or non-biological sample is obtained from a human with a viral respiratory disease. In some embodiments, the biological or non-biological sample is obtained from a human with a coronavirus infection. In some embodiments, the biological or non-biological sample is obtained from a human with a SARS-CoV-2 infection.

In some embodiments, the biological or non-biological sample is an analysis (e.g., test) sample or a control sample (e.g., a positive control, negative control, and/or blank control).

In some embodiments, the biological or non-biological sample comprises nucleic acids (e.g., RNA or DNA). In some embodiments, the nucleic acids included in the biological or non-biological sample comprise any of the embodiments described herein. See, for example, Definitions: Nucleic acids, and Definitions: Samples.

Sequencing and Mapping.

As described above, in some implementations, the sequencing generates a plurality of nucleotide sequences that can be mapped against a plurality of reference sequences. In some embodiments, the sequencing is performed on a sample or portion thereof that has undergone a nucleic acid amplification process. Alternatively, in some embodiments, the sequencing is performed on a sample or portion thereof that has not undergone a nucleic acid amplification process. In some embodiments, nucleic acid molecules within a sample or portion thereof are fragmented prior to undergoing sequencing. Alternatively, in some embodiments, nucleic acid molecules are not fragmented prior to undergoing sequencing. Multiple different schemes may be applied to identify nucleic acid sequences within a sample.

Different types of nucleic acid molecules may undergo the same or different processing and sequencing. For example, in some embodiments, DNA molecules undergo a first sequencing process and RNA molecules undergo a second sequencing process, where the first and second sequencing processes include at least one process difference. In an example, genomic DNA such as accessible chromatin is processed according to a first sequencing method (e.g., using an assay for transposase-accessible chromatin using sequencing (ATAC-seq) method) while RNA molecules are processed according to a second sequencing method (e.g., a sequencing method that targets RNA molecules that include a polyA sequence, such as messenger RNA (mRNA) molecules). In some embodiments, different sequencing procedures are performed on the same or different samples. For example, in some embodiments, a first sequencing method to analyze a first type of nucleic acid molecule and a second sequencing method to analyze a second type of nucleic acid molecule, where the first and second sequencing methods are different and the first and second types of nucleic acid molecules are different, are performed on a same sample (e.g., at the same or different times). Alternatively or in addition, in some embodiments, a first sequencing method to analyze a first type of nucleic acid molecule is performed using a first sample and a second sequencing method to analyze a second type of nucleic acid molecule may be performed using a second sample, where the first and second sequencing methods are different, the first and second types of nucleic acid molecules are different, and the first and second samples are different. In some embodiments, the first and second samples are aliquots of a same sample.

In some embodiments, the sequencing is quantitative or approximately quantitative. Alternatively, in some embodiments, nucleic acid sequencing is qualitative and does not provide significant insight into the relative amounts of different nucleic acid molecules included within a sample.

Various sequencing schemes can be employed. For example, in some embodiments, the sequencing is sequencing by synthesis, sequencing by hybridization, sequencing by ligation, nanopore sequencing, sequencing using nucleic acid nanoballs, pyrosequencing, single molecule sequencing (e.g., single molecule real time sequencing), single cell/entity sequencing, massively parallel signature sequencing, polony sequencing, combinatorial probe anchor synthesis, SOLiD sequencing, chain termination (e.g., Sanger sequencing), ion semiconductor sequencing, tunneling currents sequencing, heliscope single molecule sequencing, sequencing with mass spectrometry, transmission electron microscopy sequencing, RNA polymerase-based sequencing, or any other method, or a combination thereof. In some embodiments, the sequencing is a sequencing technology like Heliscope (Helicos), SMRT technology (Pacific Biosciences) or nanopore sequencing (Oxford Nanopore) that allows direct sequencing of single molecules without prior clonal amplification. In some embodiments, the sequencing is performed with or without target enrichment. In some embodiments, the sequencing is Helicos True Single Molecule Sequencing (tSMS) (e.g., as described in Harris T. D. et al., Science 320:106-109 [2008]). In some embodiments, the sequencing is 454 sequencing (Roche) (e.g., as described in Margulies, M. et al., Nature 437:376-380 (2005)). In some embodiments, the sequencing is SOLiD™ technology (Applied Biosystems). In some embodiments, the sequencing is single molecule, real-time (SMRT™) sequencing technology of Pacific Biosciences.

In some embodiments, the systems and methods described herein are used with any sequencing platform, including, but not limited to, Illumina NGS platforms, Ion Torrent (Thermo) platforms, and GeneReader (Qiagen) platforms.

In some embodiments, the sequencing is performed as described in PCT Application No. PCT/US2019/060915, entitled “Directional Targeted Sequencing,” filed Nov. 12, 2019, which is hereby incorporated by reference herein in its entirety.

In some embodiments, the sequencing reaction is a whole genome sequencing reaction (e.g., shotgun workflow). In some instances, the sequencing is digital polymerase chain reaction (PCR) sequencing. In some embodiments, the sequencing reaction is a whole transcriptome sequencing reaction (e.g., RNASeq). In some embodiments, the sequencing reaction is a panel enriched sequencing reaction. In some embodiments, the panel is pathogen-specific and/or disease condition-specific. For example, in some embodiments, the panel is a respiratory virus oligo panel (RVOP).

In some embodiments, the plurality of nucleotide sequences (e.g., in nucleotide sequence data store 130) includes a first subset of nucleotide sequences that map (e.g., align) to a first reference sequence (e.g., a first genome) and a second subset of nucleotide sequences that map (e.g., align) to a second reference sequence (e.g., a second genome) (e.g., where the first genome is a reference genome of a host organism and the second genome is a reference genome of a microorganism). In some embodiments, the plurality of nucleotide sequences includes a plurality of subsets of nucleotide sequences, each respective subset of nucleotide sequences mapping to a corresponding reference sequence in a plurality of reference sequences (e.g., in reference sequence data store 132). In some such embodiments, the plurality of subsets of nucleotide sequences includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 10,000, or at least 50,000 subsets of nucleotide sequences that map to a corresponding reference sequence.

In some embodiments, the plurality of nucleotide sequences is at least 1000, at least 5000, at least 1×10⁴, at least 1×10⁵, at least 5×10⁵, at least 1×10⁶, at least 5×10⁶, at least 1×10⁷, at least 5×10⁷, at least 1×10⁸, or at least 2×10⁸ nucleotide sequences. In some embodiments, the plurality of nucleotide sequences is no more than 5×10⁸, no more than 1×10⁴, no more than 1×10⁷, no more than 1×10⁶, no more than 1×10⁵, no more than 1×10⁴, or no more than 5000 nucleotide sequences. In some embodiments, the plurality of nucleotide sequences is from 1000 to 1×10⁴, from 1×10⁴ to 8×10⁴, from 5×10⁴ to 5×10⁵, from 1×10⁵ to 1×10⁶, from 1×10⁶ to 5×10⁶, from 2×10⁶ to 1×10⁷, from 8×10⁶ to 5×10⁷, or from 1×10⁷ to 2×10⁸ nucleotide sequences. In some embodiments, the plurality of nucleotide sequences falls within another range starting no lower than 1000 nucleotide sequences and ending no higher than 5×10⁸ nucleotide sequences.

In some embodiments, the mapping of the plurality of nucleotide sequences against the plurality of reference sequences corresponding to a set of microorganisms (e.g., genomes), where the set of microorganisms comprises at least 3, at least 5, at least 10, at least 20, at least 30, at least 40, at least 50, at least 60, at least 70, at least 80, at least 90, or at least 100 microorganisms, collectively maps against at least 0.5, at least 1, at least 2, at least 3, at least 4, at least 5 or at least 6 megabases of the respective reference sequences (e.g., genomes). In some embodiments, the mapping of the plurality of nucleotide sequences against the plurality of reference sequences corresponding to the set of microorganisms collectively maps against at least 0.5, at least 0.8, at least 1, at least 1.5, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 100, at least 200, at least 500, or at least 1000 megabases of the respective reference sequences. In some embodiments, the mapping of the plurality of nucleotide sequences against the plurality of reference sequences corresponding to the set of microorganisms collectively maps against no more than 2000, no more than 1000, no more than 500, no more than 100, no more than 80, no more than 60, no more than 40, no more than 20, no more than 10, no more than 5, no more than 3, no more than 2, or no more than 1 megabases of the respective reference sequences. In some embodiments, the mapping of the plurality of nucleotide sequences against the plurality of reference sequences corresponding to the set of microorganisms collectively maps against from 0.5 to 10, from 1 to 6, from 2 to 5, from 4 to 15, from 8 to 20, from 12 to 30, from 10 to 60, from 20 to 100, from 75 to 500, from 100 to 1000, from 300 to 800, or from 500 to 2000 megabases of the respective reference sequences. In some embodiments, the mapping of the plurality of nucleotide sequences against the plurality of reference sequences corresponding to the set of microorganisms collectively maps against another range of megabases of the respective reference sequences starting no lower than 0.5 megabases and ending no higher than 2000 megabases.

In some embodiments, the result set further includes a plurality of nucleotide sequences mapped (e.g., aligned) to a human reference genome. Accordingly, in some embodiments, the mapping of the plurality of nucleotide sequences against a plurality of reference sequences, where the plurality of reference sequences includes a set of reference sequences corresponding to a set of microorganisms (e.g., at least 3, at least 5, at least 10, at least 20, at least 30, at least 40, at least 50, at least 60, at least 70, at least 80, at least 90, or at least 100 microorganisms) and a human reference genome, collectively maps against at least 1, at least 5, at least 10, at least 20, at least 30, at least 40, at least 50, at least 60, at least 70, at least 80, at least 90, at least 100, at least 500, at least 1000, or at least 3000 megabases. In some embodiments, the mapping of the plurality of nucleotide sequences against a plurality of reference sequences, where the plurality of reference sequences includes a set of reference sequences corresponding to a set of microorganisms and a human reference genome, collectively maps against no more than 5000, no more than 3000, no more than 1000, no more than 500, no more than 100, no more than 50, no more than 10, or no more than 5 megabases. In some embodiments, the mapping of the plurality of nucleotide sequences against a plurality of reference sequences, where the plurality of reference sequences includes a set of reference sequences corresponding to a set of microorganisms and a human reference genome, collectively maps against from 1 to 10, from 2 to 20, from 15 to 60, from 40 to 200, from 150 to 800, or from 500 to 5000 megabases. In some embodiments, the mapping of the plurality of nucleotide sequences against a plurality of reference sequences, where the plurality of reference sequences includes a set of reference sequences corresponding to a set of microorganisms and a human reference genome, collectively maps against another range of megabases starting no lower than 1 megabase and ending no higher than 5000 megabases.

In some embodiments, the analysis comprises preprocessing and/or pre-sorting of the sequencing data. In some embodiments, pre-sorting includes sorting each nucleotide sequence obtained from the sequencing of the biological or non-biological sample into one or more bins, where each bin corresponds to a different microorganism, depending on the likelihood that the nucleotide sequence originated from the respective microorganism. Each nucleotide sequence is then mapped (e.g., using a k-mer alignment and/or a full alignment) to one or more reference sequences (e.g., genomes) corresponding to different microorganisms. In some embodiments, the analysis is performed using an analysis pipeline. Methods of mapping nucleotide sequences obtained from sequencing nucleic acids are provided in, for example, Flygarc et al., 2016, “Taxonomer: an interactive metagenomics analysis portal for universal pathogen detection and host mRNA expression profiling,” Genome Biology 17:111; U.S. patent application Ser. No. 15/724,476, entitled “Methods and Systems for Multiple Taxonomic Classification,” filed Oct. 4, 2017, and U.S. Patent Application No. 62/723,384, entitled “Methods and Systems for Providing Sample Information.” filed Aug. 27, 2018, each of which is hereby incorporated by reference in its entirety. Other methods of mapping nucleotide sequences to a reference sequence are possible, as will be apparent to one skilled in the art. See, for example, Roumpeka et al., 2017, “A Review of Bioinformatics Tools for Bio-Prospecting from Metagenomic Sequence Data,” Front. Genet. 8:23, doi: 10.3389/fgene.2017.00023, which is hereby incorporated herein by reference in its entirety.

Review Portal.

In some embodiments, the nucleic acid sequencing data (e.g., nucleotide sequence data store 130) prepared for identifying the presence of a subset of microorganisms and/or antimicrobial resistance markers in a biological or non-biological sample (e.g., sample 304) comprises output or results data from the sequencing and/or mapping (e.g., result set 122), which can be performed, as described above, using any sequencing and/or mapping method as will be apparent to one skilled in the art.

In some embodiments, some or all of the nucleic acid sequencing data is accessed via a system (e.g., in accordance with the example system 100 embodiments described above) for review and/or visualization. In some embodiments, the review and/or visualization is performed on the display of a computer. In some embodiments, the review and/or visualization is performed using a cloud-based interface such as an online portal.

In some embodiments, some or all of the nucleic acid sequencing data is transmitted from a first system for performing sequencing and/or mapping analysis, to a second system (e.g., in accordance with the example system embodiments described above) for performing review and/or visualization. In some embodiments, some or all of the nucleic acid sequencing data is transmitted from a first system for performing sequencing and/or mapping analysis, to a cloud-based interface, such as an online portal for performing the review and/or visualization. In some embodiments, the sequencing and/or mapping analysis is performed using an analysis pipeline.

In some embodiments, the method comprises generating an alert when no nucleic acid sequencing data is available to perform the method (e.g., receiving an email notification when data upload fails).

In some embodiments, the review and/or visualization is performed on the same system as the sequencing and/or mapping analysis, where the sequencing, mapping, review, and/or visualization of some or all of the nucleic acid sequencing data is performed within an analysis workflow. In some embodiments, the sequencing, mapping, review, and/or visualization is performed at a cloud-based interface such as an online portal comprising an analysis pipeline. In some embodiments, the sequencing, mapping, review, and/or visualization is performed using a software program (e.g., Explify). See Example 1 (Examples, below). See, for example, IDbyDNA, 2019, “Explify Software v1.5.0 User Manual,” Document No. TH-2019-200-006, pp. 1-44, which is hereby incorporated by reference herein in its entirety.

Dashboard.

In some embodiments, the method further facilitates review of nucleic acid sequencing data prepared for identifying the presence of a subset of microorganisms and/or antimicrobial resistance markers in a biological or non-biological sample 304 (e.g., from a subject), where the biological or non-biological sample is selected from a plurality of biological or non-biological samples (e.g., from the same subject or from a plurality of subjects). In some embodiments, the method facilitates review of nucleic acid sequencing data in a plurality of biological or non-biological samples, where each respective biological or non-biological sample corresponds to a respective subject in a plurality of subjects. In some embodiments, the method facilitates review of nucleic acid sequencing data in a plurality of biological or non-biological samples, where the plurality of biological or non-biological samples includes a biological or non-biological sample obtained from a subject and one or more control samples.

In some embodiments, the plurality of biological or non-biological samples (e.g., samples 304) are displayed on a display (e.g., of a system for review and visualization). In some embodiments, the display is provided in a system for review and visualization (e.g., system 100), and the one or more biological or non-biological samples are displayed on a dashboard (e.g., results dashboard 302). In some embodiments, the plurality of biological or non-biological samples are displayed as a sample queue (e.g., sample queue 306).

In some embodiments, the one or more biological or non-biological samples comprises a list of pending samples (e.g., a sample queue comprising one or more samples awaiting or undergoing review).

In some embodiments, the one or more biological or non-biological samples comprises one or more batches 310 (e.g., batch 310-1), where each batch includes one or more samples 304. For example, in some embodiments, each sample in a respective plurality of samples in a batch is sequenced using the same method as every other sample in the respective plurality of samples in the batch (e.g., from the same sequencing run). In some embodiments, each sample in a respective plurality of samples in a batch is processed using the same method as every other sample in the respective plurality of samples in the batch (e.g., collected and/or prepared for sequencing at the same time and/or via matched processes). In some such embodiments, the one or more biological or non-biological samples comprises 2, 3, 4, 5, 6, 7, 8, 9, 10, or more than 10 batches, where each batch includes 1, 2, 3, 4, 5, 6, 7, 8, 9, 10, or more than 10 samples.

In some embodiments, the plurality of biological or non-biological samples comprises one or more runs 314 (e.g., run 314-1), where each respective run includes one or more batches 310, and each respective batch includes one or more samples 304. For example, in some embodiments, the plurality of samples in a respective run consists of a plurality of samples sequenced during the same sequencing run. In some such embodiments, the one or more runs comprises 2, 3, 4, 5, 6, 7, 8, 9, 10, or more than 10 runs, where each batch in each run includes 1, 2, 3, 4, 5, 6, 7, 8, 9, 10, or more than 10 batches, and each respective batch in each run includes 1, 2, 3, 4, 5, 6, 7, 8, 9, 10, or more than 10 samples.

In some embodiments, the one or more samples, one or more batches, and/or one or more runs are displayed on a system for review and visualization (e.g., a user interactive system 100 for review and visualization). In some embodiments, an index of the one or more samples, one or more batches, and/or one or more runs are displayed on a user interactive dashboard (e.g., results dashboard 302) on the system for review and visualization.

FIG. 3A illustrates an example of a user dashboard (results dashboard 302) displaying a list of biological or non-biological samples 304 (e.g., 304-1, 304-2, 304-3) in a pending sample queue 306, in accordance with some embodiments of the present disclosure. A first batch affordance is displayed for displaying a plurality of batches 308 (e.g., show/hide batches). Responsive to selection of the first batch affordance 308, a second sample affordance is displayed for displaying a plurality of samples 310 (e.g., show/hide samples). The pending status of each sample in the plurality of samples in the sample queue is represented by the review status 312 (e.g., MD Review, Final Review, etc.), indicating that the sample is awaiting or undergoing the approval represented by the review status (e.g., awaiting MD Review, awaiting Final Review, etc.).

Control Samples.

In some embodiments, the one or more control samples are obtained from the same or a different subject as the biological or non-biological sample used for identifying the presence of a subset of microorganisms and/or antimicrobial resistance markers. In some embodiments, the one or more control samples are obtained externally (e.g., using laboratory standards). In some embodiments, a control sample is a positive control sample (e.g., 304-cp), a negative control sample (e.g., 304-np), or a blank control sample (e.g., 304-blk).

For example, in some embodiments, a biological or non-biological sample is a positive control sample, where the positive control sample comprises a known, non-zero amount of nucleic acids corresponding to one or more microorganisms in the subset of microorganisms.

In some embodiments, the positive control sample is obtained from a subject with a known population of a microorganism (e.g., a pathogenic infection). In some such embodiments, the positive control sample is obtained from a subject diagnosed with an infectious disease. In some such embodiments, the positive control sample is obtained from diseased tissue in a subject diagnosed with an infectious disease.

In some embodiments, the presence of a microorganism in the positive control sample is validated by a laboratory validation technique, such as targeted enrichment sequencing, PCR, in vitro culture, immunoassays (e.g., ELISA, Western blot, chemiluminescence, etc.), serological assays and/or antimicrobial susceptibility assays.

In some embodiments, the positive control sample comprises whole or lysed microorganisms from an in vitro culture. In some embodiments, the positive control sample comprises nucleic acids isolated from one or more microorganisms in the subset of microorganisms. In some embodiments, the positive control sample comprises nucleic acids synthesized based on one or more reference sequences (e.g., complete and/or incomplete genomes) corresponding to a respective one or more microorganisms in the subset of microorganisms.

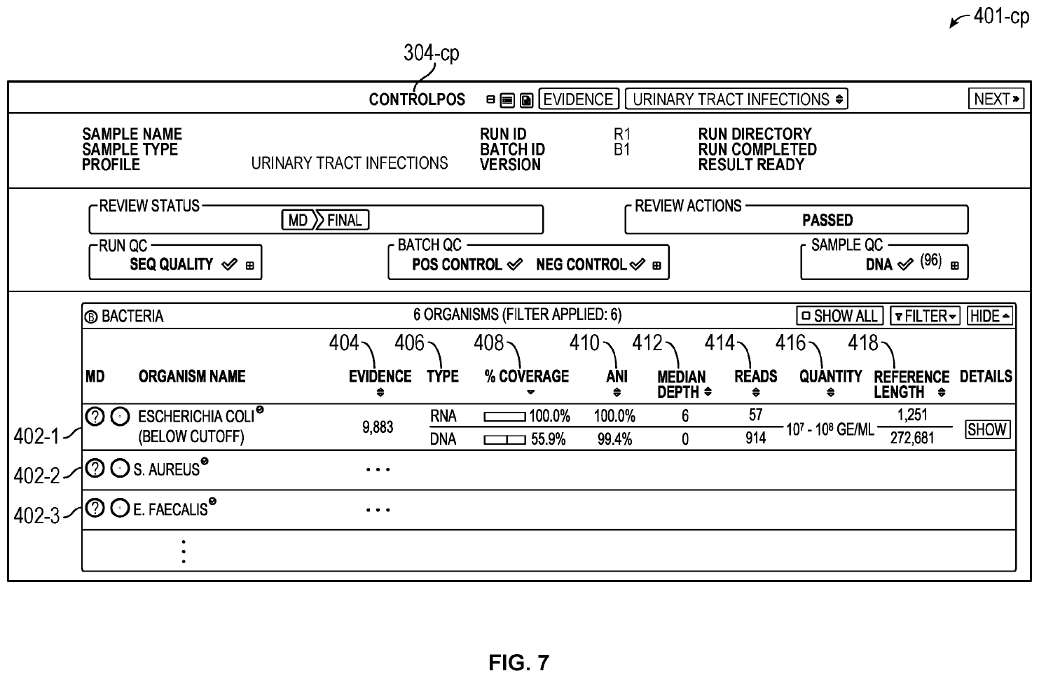

For instance, FIG. 7 illustrates an example of an analysis (e.g., customizable user interface 401-cp) of a result set 122 obtained from a sequencing reaction of nucleic acids from a positive control sample 304-cp, in accordance with some embodiments of the present disclosure. As illustrated in FIG. 7, the positive control sample 304-cp is characterized by robust detection of a plurality of microorganisms 402 (e.g., 402-1, 402-2, 402-3, etc.). For example, the positive control exhibits a high percentage of coverage 408 of the reference genomes of five different microorganisms detected in the positive control, based on the alignment of the plurality of nucleotide sequences in the positive control sample to the respective reference genomes (e.g., above 99% for all RNA alignments and above 99% for 3 out of 5 DNA alignments). Average nucleotide identity (ANI) 410, which reports a measure of nucleotide-level genomic similarity between the coding regions of two reference sequences, also confirmed with a high level of certainty that the positive control sample included nucleic acids corresponding to the one or more microorganisms (e.g., ANI of above 99% for all nucleic acid types against all detected microorganisms).

In some embodiments, a biological or non-biological sample is a negative control sample, where the negative control sample does not include nucleic acids corresponding to a microorganism in the subset of microorganisms. In some embodiments, the negative control sample is obtained from a healthy subject. In some embodiments, the negative control sample is obtained from a healthy tissue in a subject diagnosed with an infectious disease. In some embodiments, the absence of one or more microorganisms in the subset of microorganisms in the negative control sample is validated by a laboratory validation technique, such as targeted enrichment sequencing, PCR, in vitro culture, immunoassays (e.g., ELISA, Western blot, chemiluminescence, etc.), serological assays and/or antimicrobial susceptibility assays.

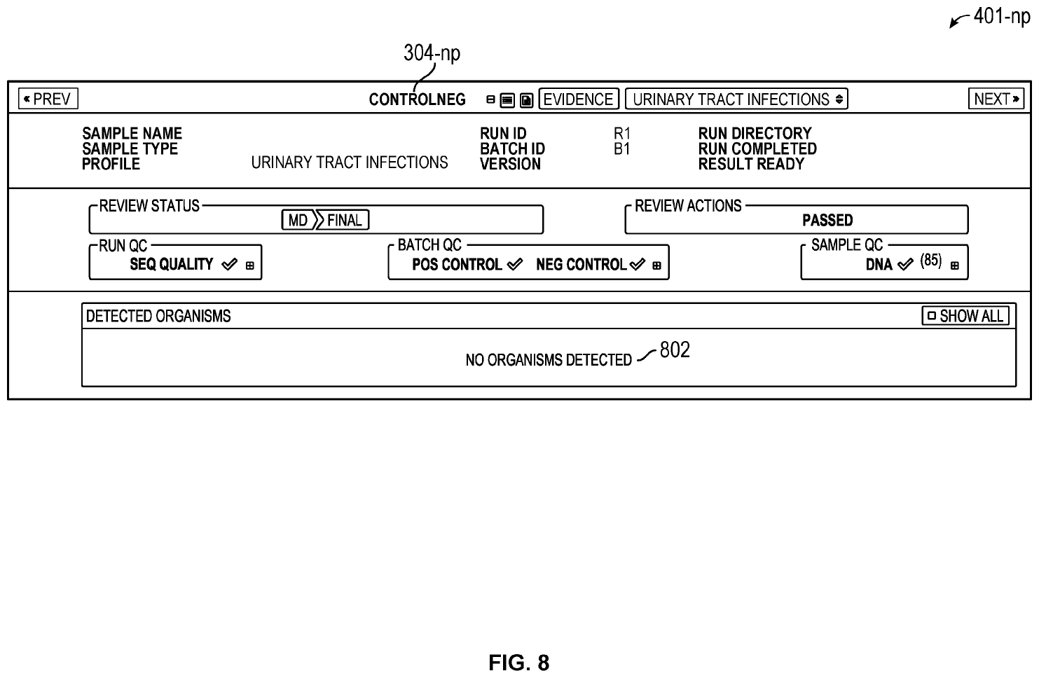

For instance, FIG. 8 illustrates an example of an analysis (e.g., customizable user interface 401-cn) of a result set 122 obtained from a sequencing reaction of nucleic acids from a negative control sample 304-cn, in accordance with some embodiments of the present disclosure. In contrast with the positive control sample illustrated in FIG. 7, the negative control sample 304-cn failed to detect any microorganisms (802). Notably, passing scores for quality control checks at the sample (420-3), batch (420-2), and run level (420-1) (e.g., represented by green check marks) indicated that the sequencing and mapping processing prior to microorganism detection analysis were performed successfully. providing an additional layer of confidence in the analysis of the negative control sample result set.

In some embodiments, a biological or non-biological sample is a blank control sample, where the blank control sample does not include nucleic acids corresponding to a microorganism in the subset of microorganisms. In some embodiments, the blank control sample does not comprise biological material. In some embodiments, the blank control sample comprises one or more reagents used for processing the positive control sample and/or the negative control sample (e.g., reagents for sample collection, sample storage, pre-processing, nucleic acid isolation, and/or sequencing). In some embodiments, the blank control sample is water.

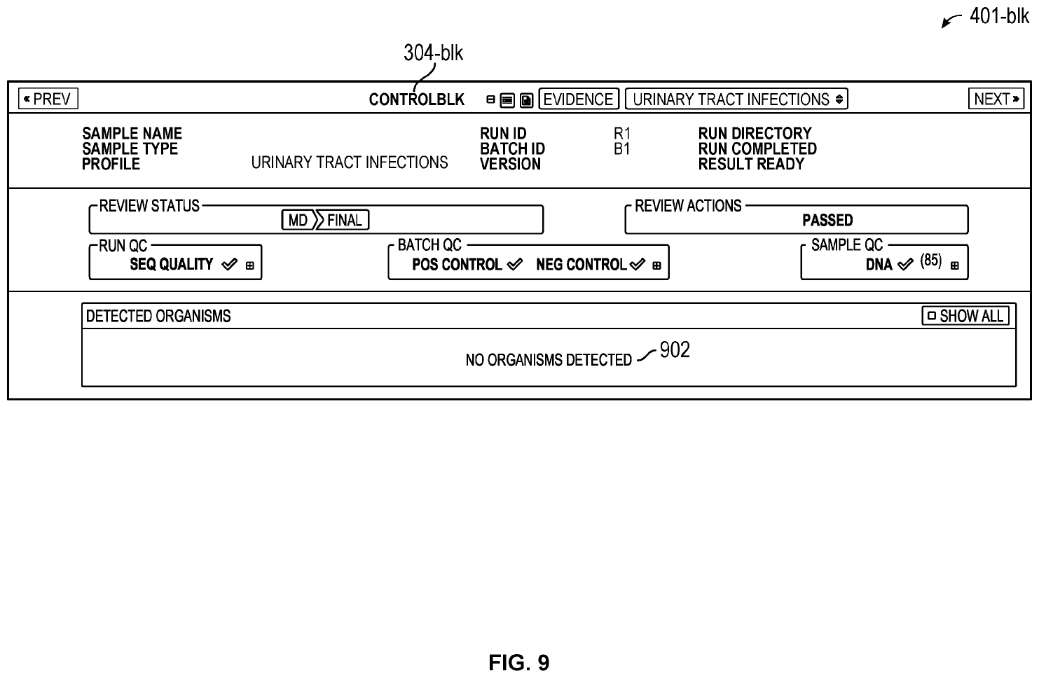

For instance, FIG. 9 illustrates an example of an analysis (e.g., customizable user interface 401-blk) of a result set 122 obtained from a sequencing reaction of nucleic acids from a blank control sample 304-blk, in accordance with some embodiments of the present disclosure. As observed with the negative control sample illustrated in FIG. 8, the blank control sample 304-blk failed to detect any microorganisms (902). Additionally, passing scores for quality control checks at the sample (420-3), batch (420-2), and run level (420-1) (e.g., represented by green check marks) indicated that the sequencing and mapping processing prior to microorganism detection analysis were performed successfully, providing an additional layer of confidence in the analysis of the blank control sample result set.

In some embodiments, a first control sample and a second control sample are matched samples. For example, in some embodiments, a positive control sample and a negative control sample are obtained from a diseased tissue and a healthy tissue from the same subject, respectively. In some embodiments, a positive control sample and a negative control sample are obtained from a subject diagnosed with an infectious disease and a healthy subject from the same cohort, respectively (e.g., in a clinical study).

In some embodiments, a first control sample and a second control sample are process matched. For example, in some embodiments, a positive control sample and a negative control sample are prepared using the same process, including the reagents, equipment, processing times, and/or operator or technician used to perform the method, as well as matching workflows for sequencing, mapping, and/or preprocessing. Similarly, in some embodiments, a positive and/or negative control sample is process matched with a blank control sample, such as where the blank control sample comprises the reagents used to process the positive and/or negative control sample, and is subjected to a workflow that matches the processing workflow for the positive and/or negative control sample.

Analysis Samples.

In some embodiments, a biological or non-biological sample is an analysis sample (e.g., a test sample where the presence of microorganisms is unknown and/or under investigation). For example, in some embodiments, a biological or non-biological sample is a clinical sample, a diagnostic sample, an environmental sample, a consumer quality sample, a food sample, a biological product sample, a microbial testing sample, a tumor sample, a forensic sample and/or a laboratory or hospital sample. In some embodiments, biological or non-biological sample is obtained from a human or an animal. In some embodiments, a biological or non-biological sample is a sample from a patient undergoing a treatment.

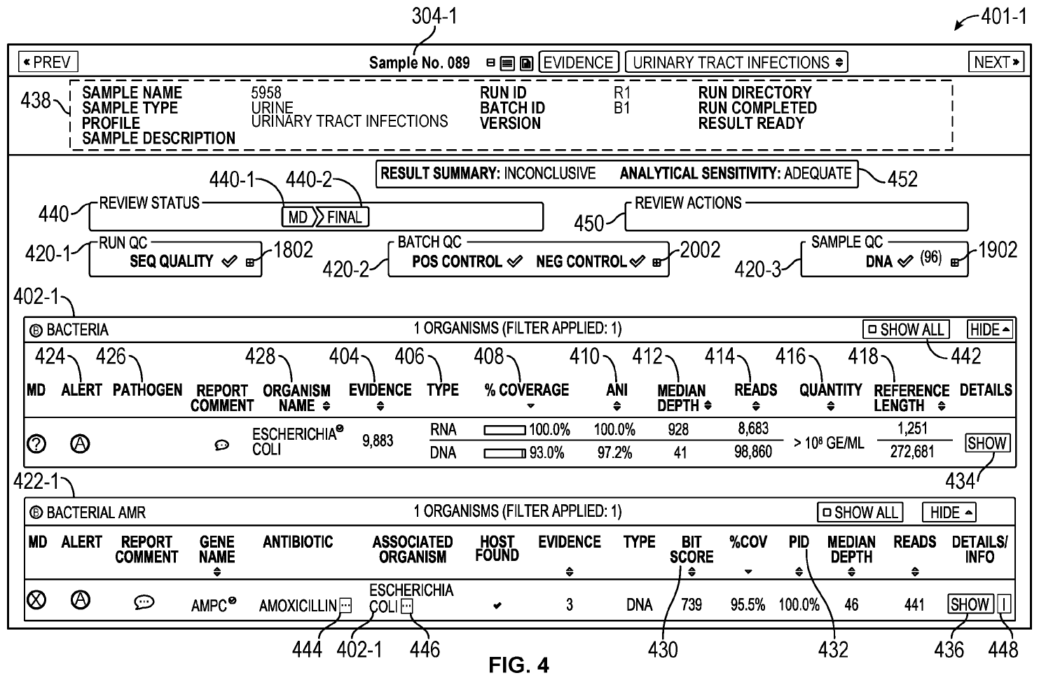

FIG. 4 illustrates an example of an analysis of a result set 122 obtained from a sequencing reaction of nucleic acids from an analysis sample 304-1, in accordance with some embodiments of the present disclosure. Features included in the analysis of the result set 122 include detection of a bacterial microorganism 402-1 (e.g., Escherichia coli) and detection of a bacterial antimicrobial resistance gene 422-1 (e.g., ampC). Validation of the detected microorganism and antimicrobial resistance gene, however, will depend on an assessment of a plurality of sequencing statistics 128 and/or a plurality of mapping statistics 126 for the respective microorganism. In some embodiments, the assessment and subsequent validation is performed automatically (e.g., by a first customizable diagnostic template 138-1). In some embodiments, the assessment and subsequent validation is performed by user interaction (e.g., by a reviewer). Further details regarding features presented in an analysis of a result set obtained from a sequencing reaction of nucleic acids from an analysis sample are discussed below (see, Features of the analysis; and Viewing features).

Receiving Requests for Analysis.

Referring again to FIG. 2 at Block 202, the method further comprises receiving a request to display an analysis (e.g., customizable user interface 401-1) of a result set 122 obtained from a sequencing reaction of nucleic acids 130 from the biological or non-biological sample. The result set includes (i) a plurality of sequencing statistics 128 from the sequencing reaction, (ii) a plurality of nucleotide sequences mapped 124 against the reference sequences (e.g., complete and/or incomplete genomes) of a set of microorganisms 132, where the set of microorganisms comprises at least 3, at least 5, at least 10, at least 20, at least 30, at least 40, at least 50, at least 60, at least 70, at least 80, at least 90, or at least 100 microorganisms, and (iii) for each respective microorganism in the set of microorganisms, a corresponding plurality of mapping statistics 126 for the mapping of respective nucleotide sequences to the reference sequence of the respective microorganism.

For instance, in some embodiments, the result set is an output of an analysis pipeline. In some embodiments, the result set is data generated from an analysis of sequencing data. In some embodiments, the result set is data generated from an analysis of a mapping of nucleotide sequences to a reference sequence (e.g., of nucleic acid sequencing data to a reference genome). In some embodiments, the result set is obtained from an analysis software (e.g., BaseSpace, BasePair, Strand-NGS, CLC Genomics Workbench, etc.).

In some embodiments, the receiving the request includes receiving log-in credentials for a user; displaying, on the display, an index of biological or non-biological samples for the user (e.g., a results dashboard 302 and/or a sample queue 306); and detecting selection of a respective biological or non-biological sample 304 from the index. In some embodiments, the log-in credentials are for an organization (e.g., a hospital, diagnostic testing company, research institution, etc.). In some embodiments, the log-in credentials are for an individual (e.g., a patient, a medical practitioner, a primary physician, a medical director (2502-3), a reviewer (2502-4), a research technician, a research supervisor, etc.).

In some embodiments, the receiving the request further includes receiving log-in credentials for an administrator account user (e.g., 2502-1): displaying, on the display, an index (e.g., 2102) of affordances for administrator action (e.g., an administrator dashboard 2108) and an index of biological or non-biological samples for the user (e.g., 2106); and detecting selection of an affordance for administrator action and/or a respective biological or non-biological sample from the index.

In some embodiments, the receiving the request further includes receiving log-in credentials for a demo account user (e.g., 2502-2); displaying, on the display, an index of affordances for demo (e.g., for testing and/or trialing); and detecting selection of an affordance for testing and/or trial purposes.

In some embodiments, the receiving the request includes receiving log-in credentials for a plurality of users (e.g., 2402-1, 2402-2, 2402-3, etc.). In some embodiments, a plurality of requests can be received simultaneously from a plurality of users. In some embodiments, only one user at a time can submit a request by entering log-in credentials. In some embodiments, log-in credentials include a username and/or a password. In some embodiments, log-in credentials include an email address.

In some embodiments, the detecting selection of a respective biological or non-biological sample comprises detecting a selection of the respective sample from an index of samples to be displayed (e.g., selection of a sample from a list of samples 304 in a pending queue 306 displayed on a user interactive results dashboard 302). In some embodiments, the receiving a request to display an analysis of a respective sample (e.g., a sample 304) comprises detection of an affordance for performing a review of the analysis (e.g., review affordance 332).

In some such embodiments, the receiving the request includes displaying, on the display, an index of sets (e.g., batches 310 and/or runs 314) of samples for the user; and detecting selection of a respective set (e.g., batch 310 and/or run 314) of samples from the index. For instance, in some embodiments, the method comprises receiving a selection of a respective batch in a plurality of batches displayed on an index of batches and/or runs.

In some embodiments, the display includes, for each sample in the index of samples for the user, a sample summary comprising an indication of a run quality control metric, an indication of a sample quality control metric, and an indication of a subset of the set of microorganisms (e.g., selected by an analysis of the result set). For example, FIG. 3A illustrates a user interactive results dashboard 302 comprising a list of samples 306. Each sample 304 in the list of samples comprises a summary 318. Each summary for the respective sample includes an indication of a run quality control metric 320 (e.g., pass indicated by a check mark, fail indicated by no check mark or an X-mark), an indication of a sample quality control metric 322 (e.g., pass indicated by a check mark; fail indicated by no check mark or an X-mark), and an indication of a subset of the set of microorganisms 324. In some embodiments, the indication of the subset of the set of microorganisms indicates a class of a microorganism detected in the sample (e.g., B: bacteria; F: fungi; V: virus; P: parasite). In some embodiments, the indication of the subset of the set of microorganisms indicates a number of microorganisms detected in the sample and/or, for each class of microorganism detected in the sample, the number of detected microorganisms in the respective class. In some embodiments, summary for the respective sample further includes an indication of a presence or absence of an antimicrobial resistance marker such as an AMR gene (e.g., R).

In some embodiments, selection of a sample (e.g., 304-1) generates an overview of the results set (e.g., customizable user interface 401-1) generated by, e.g., an analysis pipeline, indicating the number of microorganisms, if any, detected in the sample.

Microorganisms.

In some embodiments, a microorganism is a single-celled organism and/or a colony of single-celled organisms. In some embodiments, a microorganism is eukaryotic or prokaryotic. In some embodiments, a microorganism is a pathogen (e.g., disease-causing), such as a human, animal, or plant-infective pathogen. In some embodiments, a microorganism in the set of microorganisms is any one of the microorganisms described herein (See, Definitions: “Microorganisms,” above). In some embodiments, a microorganism in the set of microorganisms is any one of the microorganisms selected from a database, including but not limited to NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC. TBDB, PlasmoDB, the Microbial Genome Database (MBGD), and/or the Microbial Rosetta Stone Database.

In some embodiments, the set of microorganisms comprises at least 3, at least 4, at least 5, at least 6, at least 7, at least 8, at least 9, at least 10, at least 20, at least 30, at least 40, at least 50, at least 60, at least 70, at least 80, at least 90, at least 100, or at least 500 microorganisms. In some embodiments, the set of microorganisms is at least 1000, at least 2000, at least 5000, at least 7500, at least 10,000, at least 20,000, at least 30,000, or at least 50,000 microorganisms. In some embodiments, the set of microorganisms comprises no more than 80,000, no more than 50,000, no more than 10,000, no more than 1000, no more than 500, no more than 100, no more than 50, or no more than 20 microorganisms. In some embodiments, the set of microorganisms comprises from 3 to 10, from 8 to 30, from 20 to 80, from 75 to 200, from 100 to 1000, from 800 to 3000, from 2500 to 7500, or from 5000 to 20,000 microorganisms. In some embodiments, the set of microorganisms falls within another range starting no lower than 3 microorganisms and ending no higher than 80.000 microorganisms.

In some embodiments, a microorganism in the set of microorganisms comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 microorganisms selected from the lists provided above and/or selected from any one or more of the databases provided above. In some embodiments, a microorganism in the set of microorganisms comprises at least 100, at least 200, at least 300, at least 400, at least 500, at least 600, at least 700, at least 800, at least 900, at least 1000, at least 2000, at least 3000, at least 4000, at least 5000, at least 10,000 or at least 50,000 microorganisms selected from the lists provided above and/or selected from any one or more of the databases provided above. In some embodiments, a microorganism in the set of microorganisms comprises between 1 and 50, between 50 and 100, between 100 and 200, between 200 and 500, between 500 and 1000, between 1000 and 2000. between 2000 and 3000, between 3000 and 5000, between 5000 and 10,000, between 10,000 and 50,000, or more than 50,000 microorganisms selected from the lists provided above and/or selected from any one or more of the databases provided above.

In some embodiments, a microorganism in the set of microorganisms is a bacterium, fungus, protozoan (e.g., protozoan parasite), virus (e.g., DNA virus and/or RNA virus), and/or helminth. In some embodiments, the set of microorganisms comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 members of a respective type (e.g., taxonomic classification, genus, species, and/or strain, including bacteria, fungi, protozoa, viruses, and/or helminths) of microorganism selected from the lists provided above and/or selected from any one or more of the databases provided above. In some embodiments, the set of microorganisms comprises at least 100, at least 200, at least 300, at least 400, at least 500, at least 600, at least 700. at least 800, at least 900, at least 1000, at least 2000, at least 3000, at least 4000, at least 5000, at least 10,000, or at least 50,000 members of a respective type (e.g., taxonomic classification, genus, species, and/or strain, including bacteria, fungi, protozoa, viruses, and/or helminths) of microorganism selected from the lists provided above and/or selected from any one or more of the databases provided above. In some embodiments, the set of microorganisms comprises between 1 and 50, between 50 and 100, between 100 and 200, between 200 and 500, between 500 and 1000, between 1000 and 2000. between 2000 and 3000, between 3000 and 5000, between 5000 and 10,000, between 10,000 and 50,000, or more than 50,000 members of a respective type (e.g., taxonomic classification, genus, species, and/or strain, including bacteria, fungi, protozoa, viruses, and/or helminths) of microorganism selected from the lists provided above and/or selected from any one or more of the databases provided above.

In some embodiments, the set of microorganisms comprises one or more microorganisms selected from at least 1, at least 2, at least 3, or at least 4 of the group consisting of: bacteria, fungi, parasites, and/or viruses.

In some embodiments, the method comprises identifying the presence of a subset of microorganisms comprising at least 1 microorganism from the set of microorganisms. In some embodiments, the method comprises identifying the presence of a subset of microorganisms comprising between 1 and 10. between 10 and 20, between 20 and 30, between 30 and 40, between 40 and 50, between 50 and 100, or more than 100 microorganisms from the set of microorganisms. In some embodiments, the method comprises identifying the presence of a subset of microorganisms comprising at least 1 microorganism selected from the lists provided above and/or selected from any one or more of the databases provided above. In some embodiments, the method comprises identifying the presence of a subset of microorganisms comprising between 1 and 10, between 10 and 20, between 20 and 30, between 30 and 40, between 40 and 50, between 50 and 100, or more than 100 microorganisms selected from the lists provided above and/or selected from any one or more of the databases provided above.

In some embodiments, a microorganism in the set of microorganisms is selected from the group consisting of bacteria, fungi, viruses, and a parasite (e.g., protozoan parasite). In some embodiments, a microorganism in the set of microorganisms is a pathogen. In some embodiments, the microorganism is a coronavirus. In some embodiments, the microorganism is severe acute respiratory syndrome coronavirus (e.g., SARS-CoV-2). In some embodiments, the microorganism is an influenza virus. In some embodiments, the microorganism is an influenza A virus.

In some embodiments, the method comprises displaying, on the display, an identifier for each microorganism in the set of microorganisms. In some embodiments, the identifier comprises a scientific name, a pathogenic status (e.g., pathogenic or nonpathogenic), an annotation (e.g., a medical relevance annotation, an associated disease, an associated antimicrobial resistance gene, an associated treatment, a number of publications used as evidence, a keyword, and/or a search term), and/or a class (e.g., bacterium, fungus, parasite, or virus).

In some embodiments, the set of microorganisms represents at least 3 reference sequences, at least 5 reference sequences, at least 10 reference sequences, at least 50 reference sequences, at least 100 reference sequences, at least 1000 reference sequences, at least 1×10⁴ reference sequences, at least 5×10⁴ reference sequences, at least 1×10⁵ reference sequences, at least 1×10⁶ reference sequences, at least 2×10⁶ reference sequences, at least 5×10⁶ reference sequences. or at least 1×10⁷ reference sequences.

Accordingly, in some embodiments, the plurality of reference sequences corresponding to the set of microorganisms (e.g., at least 3, at least 5, at least 10, or at least 100 microorganisms) collectively comprises at least 0.5, at least 0.8, at least 1, at least 1.5, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 100, at least 200, at least 500, or at least 1000 megabases. In some embodiments, the plurality of reference sequences corresponding to the set of microorganisms (e.g., at least 3, at least 5, at least 10, or at least 100 microorganisms) collectively comprises no more than 2000, no more than 1000, no more than 500, no more than 100, no more than 80, no more than 60, no more than 40, no more than 20, no more than 10, no more than 5, no more than 3, no more than 2, or no more than 1 megabases. In some embodiments, the plurality of reference sequences corresponding to the set of microorganisms (e.g., at least 3, at least 5, at least 10, or at least 100 microorganisms) collectively comprises from 0.5 to 10, from 1 to 6, from 2 to 5, from 4 to 15, from 8 to 20, from 12 to 30, from 10 to 60, from 20 to 100, from 75 to 500, from 100 to 1000, from 300 to 800, or from 500 to 2000 megabases. In some embodiments, the plurality of reference sequences corresponding to the set of microorganisms (e.g., at least 3, at least 5, at least 10, or at least 100 microorganisms) collectively comprises another range of megabases of the respective reference sequences starting no lower than 0.5 megabases and ending no higher than 2000 megabases.

In some embodiments, the method further includes displaying a plurality of nucleotide sequences mapped against the reference sequences of an organism other than a microorganism. For example, in some embodiments, the method further includes displaying a plurality of nucleotide sequences mapped against a human reference genome.

In some embodiments, the mapping is performed against one microorganism reference sequence. In some embodiments, the mapping is performed against at least 3, at least 5, at least 10, at least 20, at least 50, at least 100, at least 1000, at least 10,000, or at least 50,000 microorganism reference sequences. In some embodiments, the mapping is performed against any number of reference sequences corresponding to the set of microorganisms (e.g., at least 3, at least 5, at least 10, or at least 100 microorganisms).

In some embodiments, the reference sequences of the set of microorganisms are obtained from a nucleotide sequence database. A nucleotide sequence database can be, for example, a global genome database or a microorganism-specific genome database. For example, in some embodiments, reference sequences of the set of microorganisms are obtained from NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. See, for example, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054: and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

As illustrated in FIG. 1, in some embodiments, a plurality of reference sequences corresponding to a respective plurality of microorganisms in the set of microorganisms is stored in a reference sequence data store 132. In some embodiments, the plurality of reference sequences stored in the reference sequence data store 132 is modified (e.g., reset). In some embodiments, resetting the reference sequence data store 132 retains one or more reference sequences in the plurality of reference sequences corresponding to the respective plurality of microorganisms in the set of microorganisms. In some embodiments, resetting the reference sequence data store 132 removes some or all of the reference sequences in the plurality of reference sequences corresponding to the respective plurality of microorganisms in the set of microorganisms.

In some embodiments, as described elsewhere herein, the method comprises specifying a subset of the set of microorganisms (e.g., a subset of the set of at least 3, at least 5, or at least 10 microorganisms). In some embodiments, a respective subset of the set of microorganisms is any integer value less than or equal to the number of microorganisms in the set of microorganisms. For instance, where the set of microorganisms comprises at least 10 microorganisms, a respective subset of the set of microorganisms can be 1, 2, 3, 4, 5, 6, 7, 8, 9, 10, or more than 10 microorganisms, up to the number of microorganisms in the set of microorganisms. In another example, where the set of microorganisms comprises at least 3 microorganisms, a respective subset of the set of microorganisms can be 1, 2, 3, or more than 3 microorganisms, up to the number of microorganisms in the set of microorganisms.

In some embodiments, a subset of the set of microorganisms comprises one or more microorganisms that are grouped together based on a microorganism type (e.g., taxonomic classification, genus, species, and/or strain, including bacteria, fungi, protozoa, viruses, and/or helminths) and/or an associated disease condition. In some embodiments, a subset of the set of microorganisms comprises one or more microorganisms that are grouped together based on another parameter or filtering criterion (e.g., an evidence score, AMR gene, study type, etc.). In some embodiments, a subset of the set of microorganisms comprises one or more microorganisms that are selected and/or specified by a first customizable diagnostic template to be applied to the result set, as described below (see, e.g., the sections entitled “Features of the analysis,” “Parameters for feature selection,” “Customizable analysis of presence of microorganisms,” and “Administrator control: Test profiles,” below).

In some embodiments, an antimicrobial resistance marker is a gene. In some embodiments, an antimicrobial resistance marker is a nucleic acid sequence obtained from a reference genome. In some embodiments, an antimicrobial resistance marker is any of the embodiments described herein (see Definitions: “Antimicrobial resistance markers,” above). In some embodiments, an antimicrobial resistance marker is selected from Table 1 and/or selected from one or more databases, including but not limited to the National Database of Antibiotic Resistant Organisms (NDARO), the Comprehensive Antibiotic Resistance Database (CARD), ResFinder, PointFinder, ARG-ANNOT, ARGs-OSP, PlasmoDB, the Mycology Antifungal Resistance Database (MARDy), DBDiaSNP, the HIV Drug Resistance Database, the Virus Pathogen Resource (ViPR), and/or any of the databases used for selecting one or more microorganisms, as disclosed above.

In some embodiments, the method comprises identifying at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 antimicrobial resistance markers in a biological or non-biological sample of a subject.

In some embodiments, the method comprises identifying at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 antimicrobial resistance markers listed in Table 1 and/or selected from a database as disclosed herein, in a biological or non-biological sample of a subject.

In some embodiments, the method comprises displaying, on the display, an indication of any one or more features for the respective antimicrobial resistance marker (e.g., gene identifier, gene name, intervention (drug) information, intervention (drug) classes, associated organisms, gene families, and/or resistance mechanisms).

Sequencing Statistics.

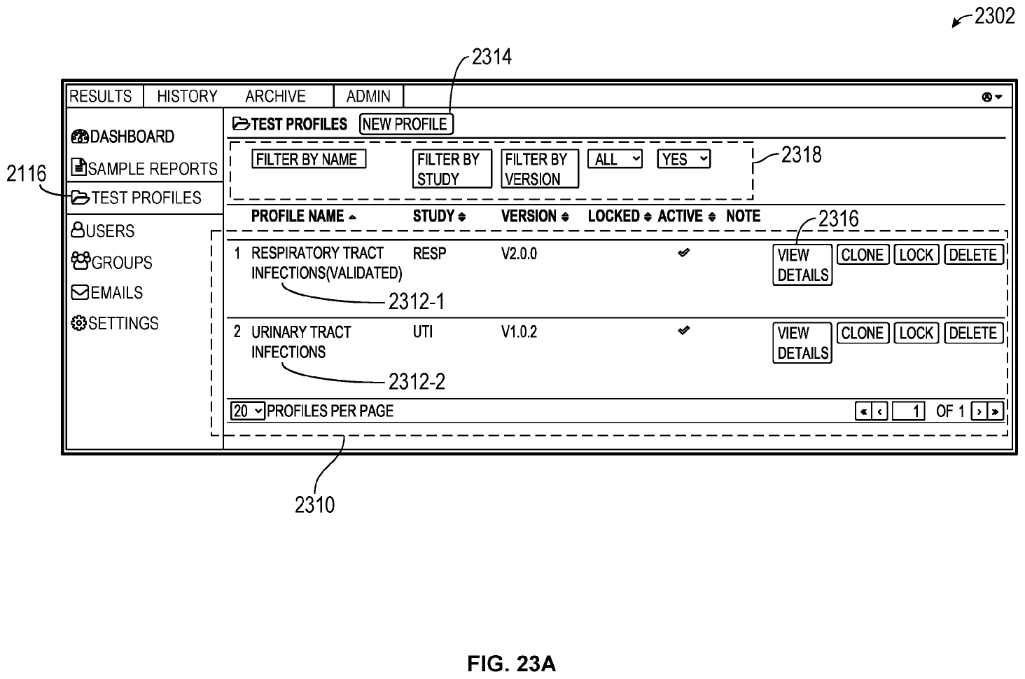

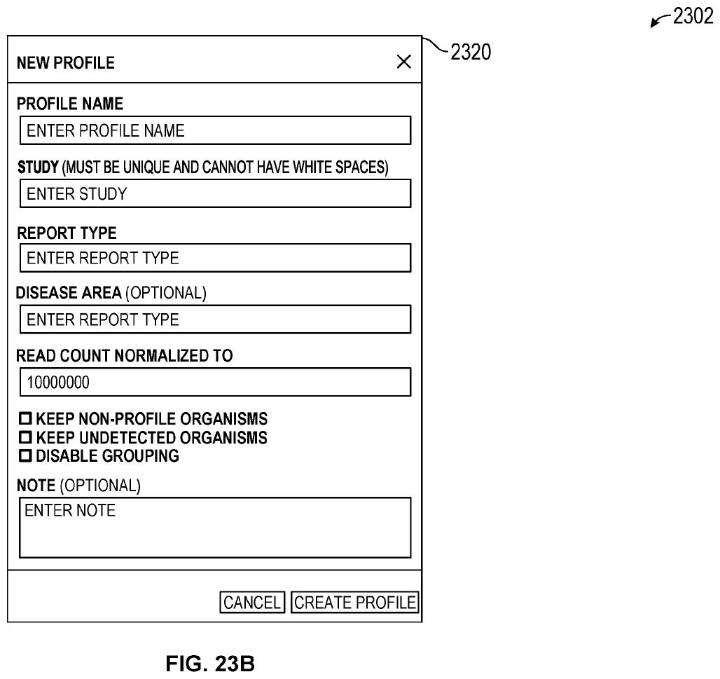

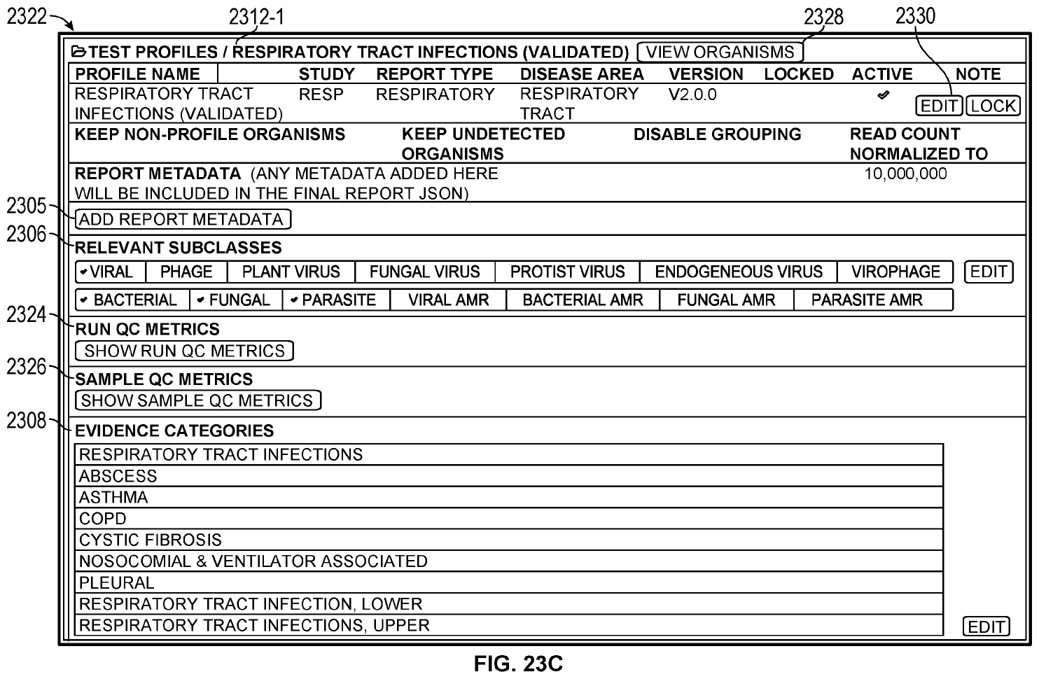

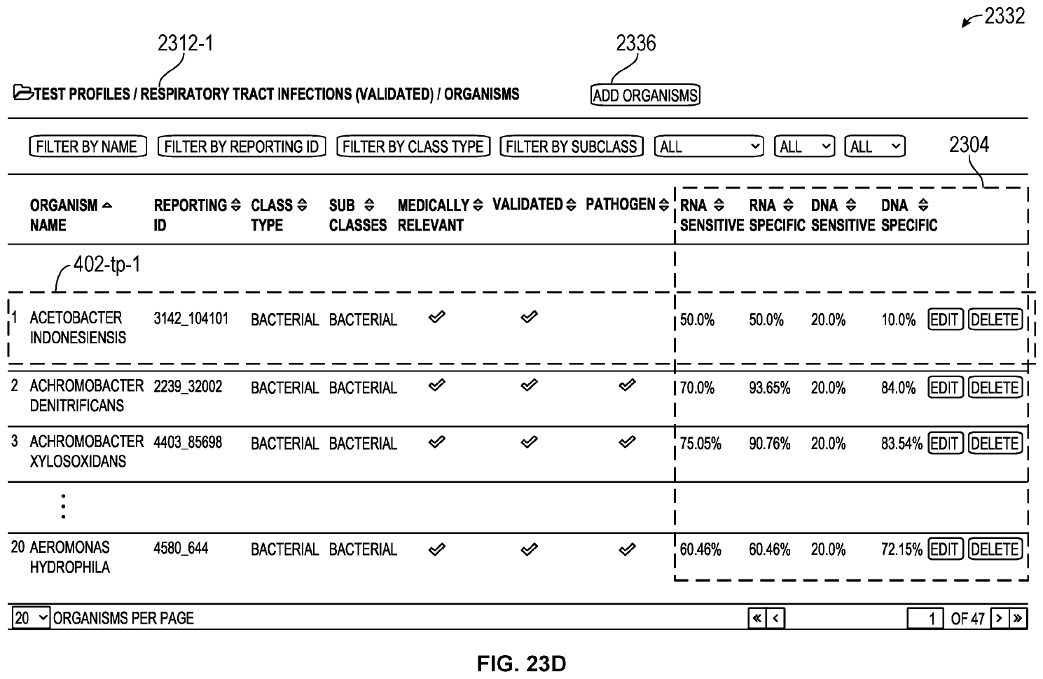

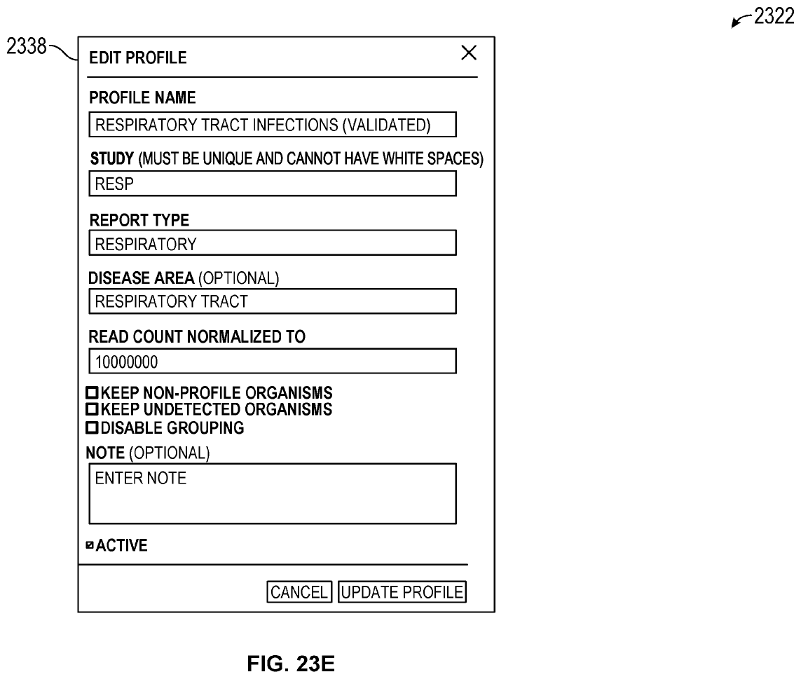

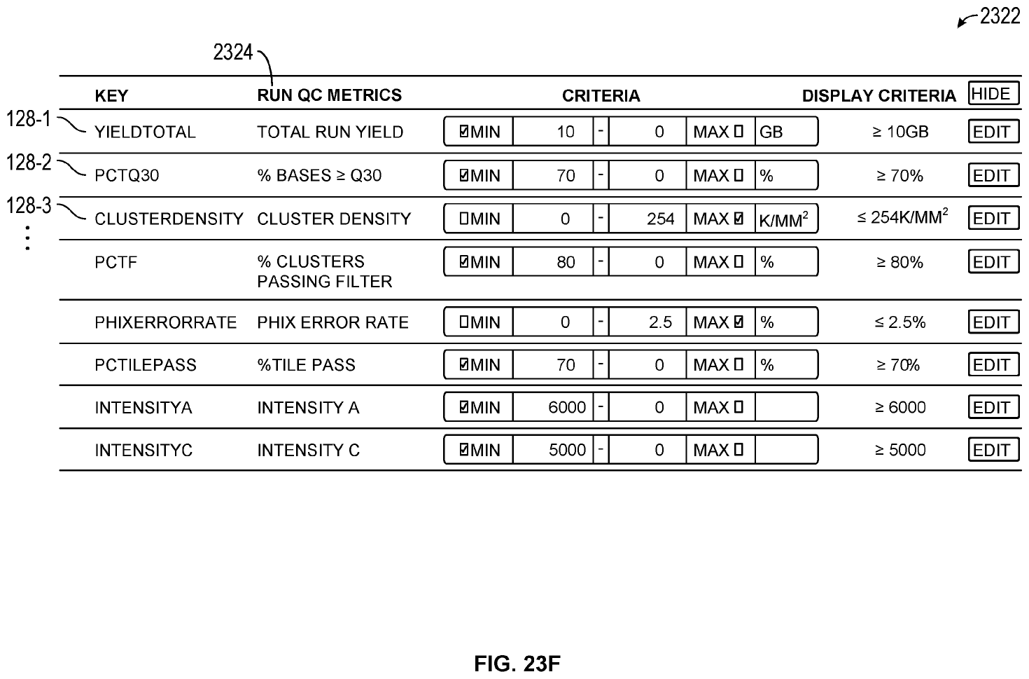

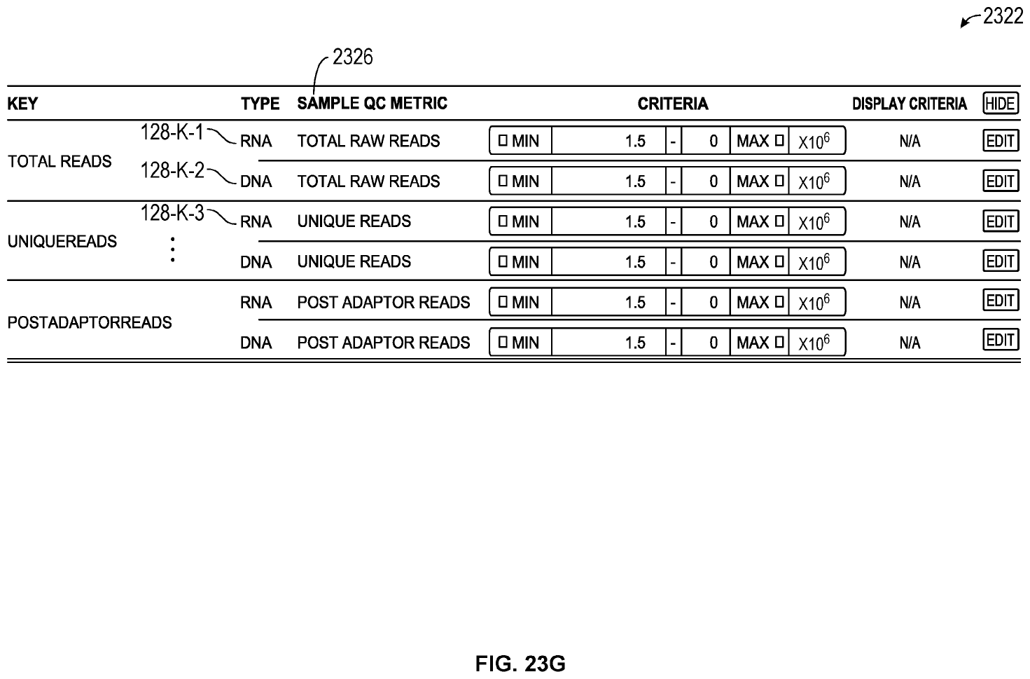

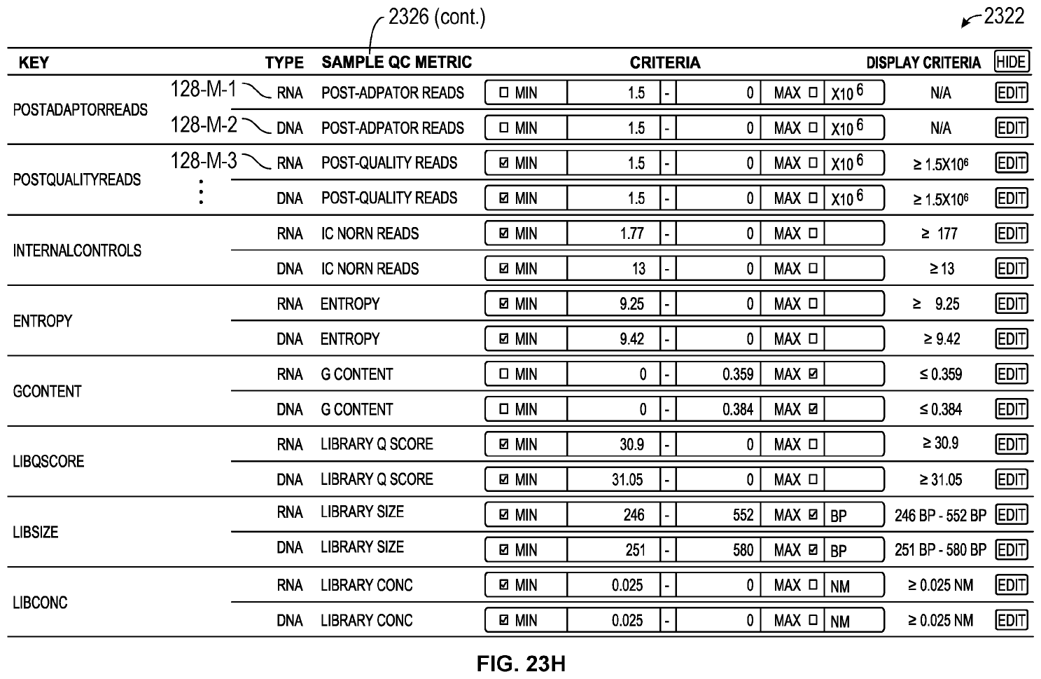

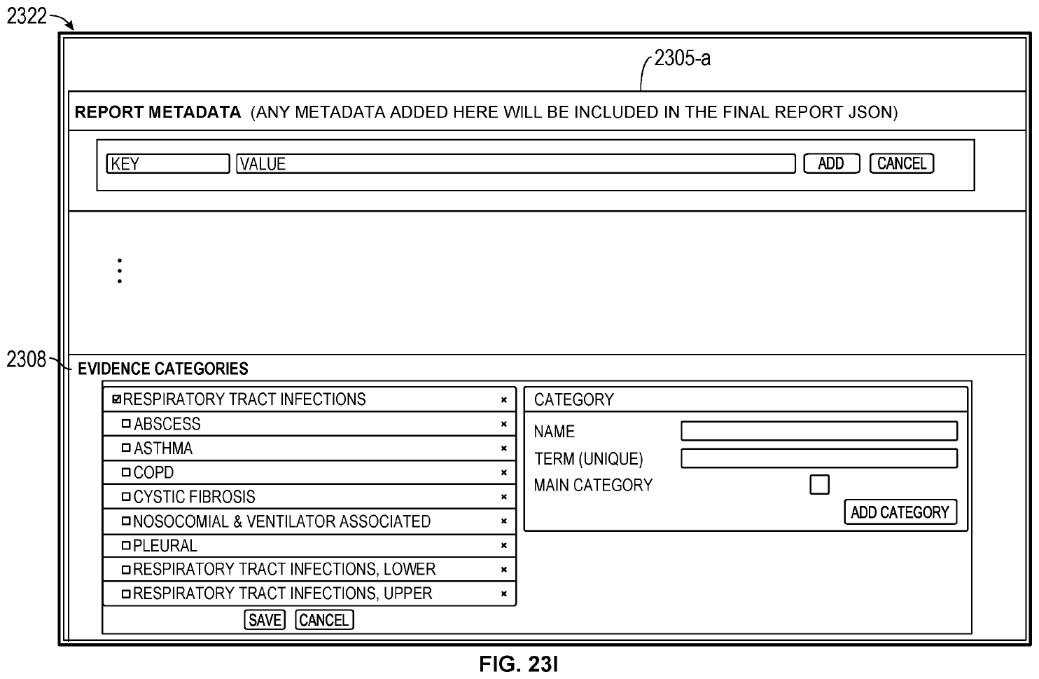

As illustrated in FIGS. 23F-H, in some embodiments, a sequencing statistic in the plurality of sequencing statistics 128 (e.g., 128-1, 128-2, 128-3, 128-K-1, 128-K-2, 128-K-3, 128-M-1, 128-M-2, 128-M-3, etc.) is a total count of nucleotide sequences in the plurality of nucleotide sequences that map to the reference sequences of the set of microorganisms (e.g., a total count of nucleotide sequences that align to the genome of one, at least one, and/or all microorganisms in the set of microorganisms).

In some embodiments, a sequencing statistic 128 is a count of unique nucleotide sequences in the plurality of nucleotide sequences that map to the reference sequences of the set of microorganisms.

In some embodiments, a sequencing statistic 128 is a count of nucleotide sequences in the plurality of nucleotide sequences that satisfy a pre-processing criterion (e.g., post-adaptor, post-quality, and/or IC norm).

In some embodiments, a sequencing statistic 128 is a quality control metric (e.g., library quality score, % Q30, and/or library Q score). For disclosure on Q scores see, for example, Illumina, 2011, “Quality Scores for Next Generation Sequencing,” Publication No. 770-2011-030, available online at illumina.com/documents/products/technotes/technote_Q-Scores.pdf; and Lopopolo and Lonie, 2017, “Sequencing Quality Control,” Oxford Genomics Centre, available online at well.ox.ac.uk/ogc/sequencing-quality-monitoring-run. The term % Q30 refers to the percentage of bases that have a Q score>=30.

In some embodiments, a sequencing statistic 128 is a measure of length for one or more nucleotide sequences in the plurality of nucleotide sequences (e.g., a read length and/or a measure of central tendency of a read length (mean, median, and/or mode)).

In some embodiments, a sequencing statistic 128 is an entropy for one or more nucleotide sequences in the plurality of nucleotide sequences. For disclosure on the entropy of a nucleic acid sequence see, for example, Schmitt and Herzel, 1997, “Estimating the Entropy of DNA Sequences,” J, theor. Biol. 1888, pp. 369-377, which is hereby incorporated by reference.

In some embodiments, a sequencing statistic 128 is a base composition for one or more nucleotide sequences in the plurality of nucleotide sequences (e.g., percent A, T, C or G content).

In some embodiments, a sequencing statistic 128 is a size of a sequencing library, a quantity of a sequencing library (e.g., a library concentration), and/or an adaptor sequence (e.g., a sequence of a sample index).

In some embodiments, the plurality of sequencing statistics 128 includes, for each sequencing statistic in the plurality of sequencing statistics, a comparison of the respective result set obtained from the respective sequencing reaction to one or more stored result sets. In some embodiments, the comparison is a distribution. For example, in some embodiments, the plurality of sequencing statistics includes a distribution plot comprising values for a sequencing statistic across a plurality of analyses and/or a plurality of samples obtained from a run history. In some such embodiments, the distribution plot illustrates the position of the current run in the distribution, thus indicating a relative quality of the current run compared to previous runs. In some embodiments, the distribution plot comprises a distribution of nucleic acid reads (e.g., RNA and/or DNA) across a plurality of samples comprising one or more control samples, where the distribution illustrates the position of the current run in the distribution, thus indicating a relative quality of the current run compared to control samples.

Other non-limiting examples of sequencing statistics include, for each nucleotide base, a count of the respective nucleotide base for each respective nucleotide sequence in the plurality of nucleotide sequences (e.g., a base composition). In some embodiments, the count of a respective nucleotide base in each respective nucleotide sequence in the plurality of nucleotide sequences is performed using RNA. In some embodiments, the count of a respective nucleotide base in each respective nucleotide sequence in the plurality of nucleotide sequences is performed using DNA.

Mapping Statistics.

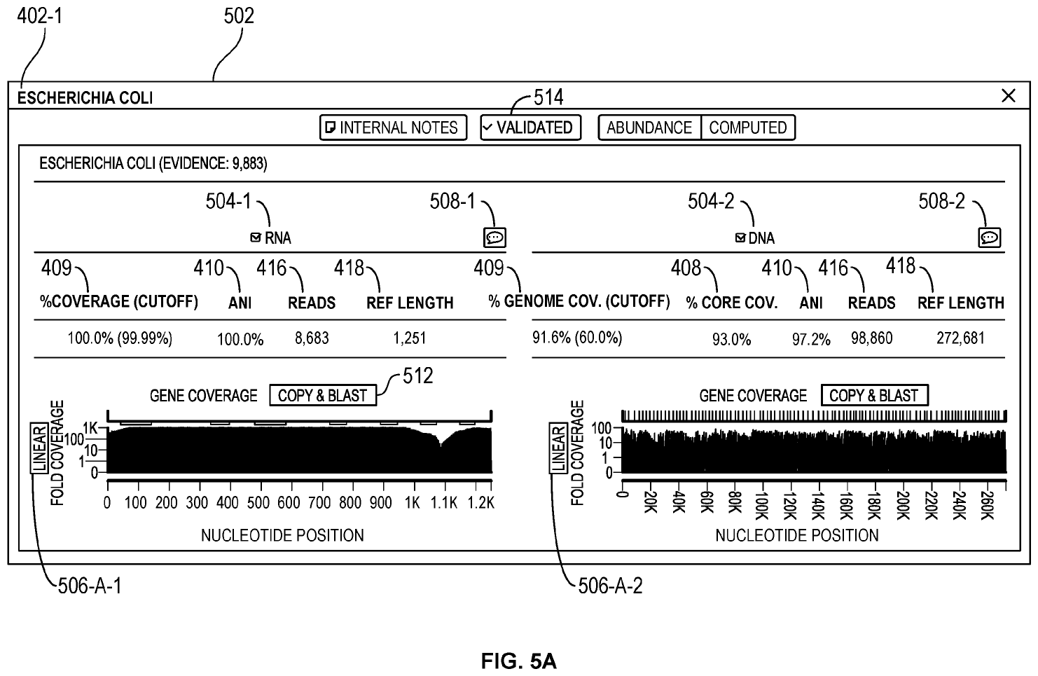

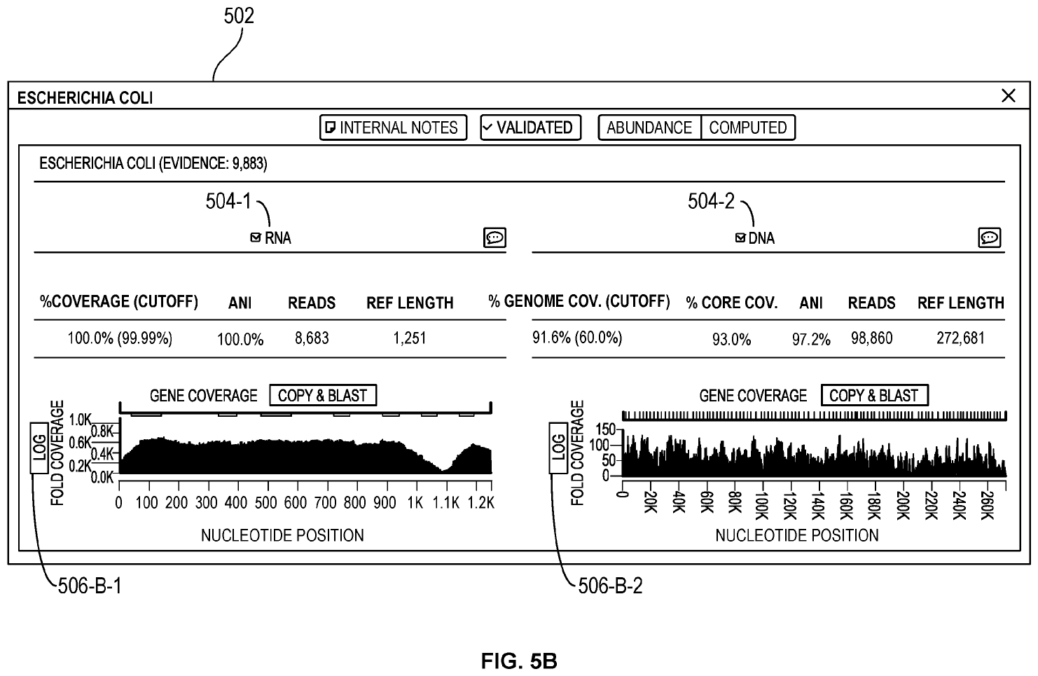

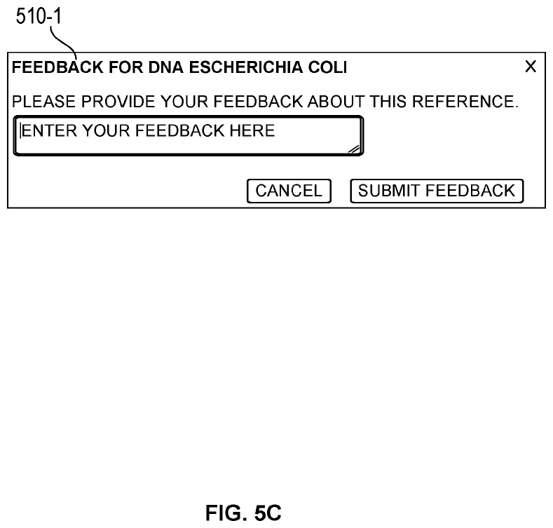

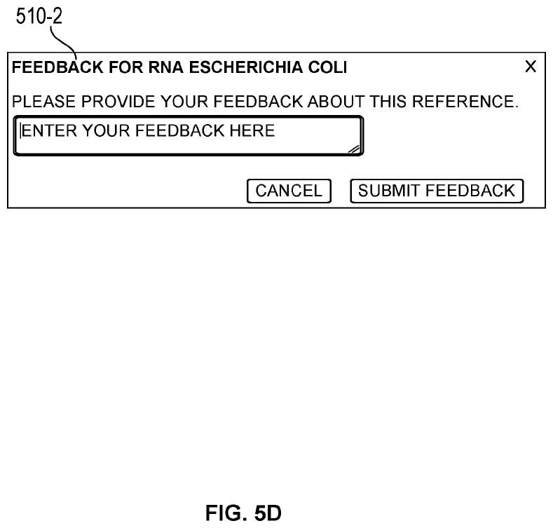

As illustrated in FIGS. 4 and 5A-D, in some embodiments, a mapping statistic in the corresponding plurality of mapping statistics 126 is an alert status 424 (e.g., N: no call; A: alert. C: critical), a pathogen status 426, an organism name 428, and/or an organism class (e.g., B: bacterium: F: fungus; V: virus; P: parasite).

In some embodiments, a mapping statistic is an annotation frequency (e.g., a medical relevance annotation, an associated disease, an associated antimicrobial resistance gene, an associated treatment, a number of publications used as evidence, a keyword, and/or a search term). For example, in some embodiments, an annotation indicating “evidence” (e.g., 404) is a number of times the microorganism is reported in a database, including publications. scientific or medical journal articles, abstracts, and/or presentations. In some embodiments, an annotation indicating “evidence” is a frequency that a microorganism reported in a database co-occurs with a disease condition of interest that is also reported in the respective database. In some embodiments, evidence annotations are used to filter putative candidates for diagnosis and therapeutic action, such as by using a filter in the second customizable diagnostic template (e.g., a test profile).

In some embodiments, a mapping statistic is a nucleic acid type 406 (e.g., RNA and/or DNA).

In some embodiments, a mapping statistic is a coverage 408. In some embodiments, coverage refers to a percent coverage of the mapping of the plurality of nucleotide sequences against the reference sequence of the microorganism. In some embodiments, coverage is presented as a graphical representation (e.g., a plot). In some such embodiments, the coverage plot is plotted as a function of depth vector and reference strength.

In some embodiments, a mapping statistic is an average nucleotide identity 410 (e.g., ANI), a quantity of the nucleic acids from the biological or non-biological sample 416 (e.g., a quantity in genome equivalents (GE) per milliliter), a length of a genome of the respective microorganism 418 (e.g., in RNA or DNA), and/or a sequence alignment score (e.g., a bit score 430 and/or a percent sequence identity (PID) 432).

In some embodiments, the plurality of mapping statistics includes a count of nucleotide sequences that map to the reference sequence of the respective microorganism 414 (e.g., RNA and/or DNA).

In some embodiments, the plurality of mapping statistics includes a ratio of (i) a count of nucleotide sequences that map to the reference sequence of the respective microorganism and (ii) a total count of nucleotide sequences in the plurality of nucleotide sequences. For example, in some such embodiments, a mapping statistic is a measure of quantitative detection based on the relative amount of microorganism-originating nucleic acids. In some embodiments, a mapping statistic measures the proportional compositions of nucleic acids in the sample (e.g., the relative abundance of human and non-human nucleotide sequences).

In some embodiments, the plurality of mapping statistics includes a depth 412 of the mapping of respective nucleotide sequences to the reference sequence of the respective microorganism. In some embodiments, the depth of the mapping of the subset of the plurality of nucleotide sequences that maps to the reference sequence of the respective microorganism is a measure of central tendency of the depth of the mapping at a plurality of regions across the reference sequence. For example, in some such embodiments, the plurality of regions includes each base position in the reference sequence of the respective microorganism. In some embodiments, a region spans at least 1 base, at least 2 bases, at least 3 bases, at least 4 bases, at least 5 bases, at least 6 bases, at least 7 bases, at least 8 bases, at least 9 bases, at least 10 base, at least 20 bases, at least 50 bases, at least 100 bases, at least 1000 bases, at least 10,000 bases, or at least 100,000 bases. In some embodiments, the measure of central tendency is a mean, median, or mode.

In some embodiments, a mapping statistic is obtained for RNA and/or DNA. For example, as illustrated in FIG. 5A, mapping statistics can be visualized for RNA (504-1) and DNA (504-2). In some embodiments, as illustrated in FIGS. 4 and 5A-D, the customizable user interface further comprises, for each respective microorganism in the set of microorganisms, an affordance 434 for displaying one or more mapping statistics for the respective microorganism (e.g., alert status, pathogen status, organism name, evidence, type, coverage, ANI, depth, reads, quantity, and/or reference length). Example embodiments for displaying one or more mapping statistics are further described in the section entitled “Viewing features,” below. In some embodiments, the plurality of mapping statistics includes a consensus sequence for the mapping of respective nucleotide sequences to the reference sequence of the respective microorganism.

In some embodiments, the plurality of mapping statistics includes an antimicrobial resistance status 422 (e.g., 422-1) detected by determining, for the respective microorganism. a locus annotated for antimicrobial resistance, and when the mapping of the respective nucleotide sequences in the plurality of nucleotide sequences to the reference sequence for the respective microorganism at the respective locus indicates the presence of an antimicrobial resistance marker (e.g., an AMR gene), including the antimicrobial resistance marker in the subset of the plurality of mapping statistics. In some embodiments, the inclusion of the antimicrobial resistance marker (e.g., the AMR gene) in the subset of the plurality of mapping statistics is further dependent on the detection of a microorganism 402 (e.g., 402-1), in the biological or non-biological sample, that is associated with the antimicrobial resistance marker. For example, in some embodiments, an antimicrobial resistance marker will not be detected and included in the plurality of mapping statistics where a microorganism that is associated with and/or that has been reported to express the respective antimicrobial resistance marker is not also detected.

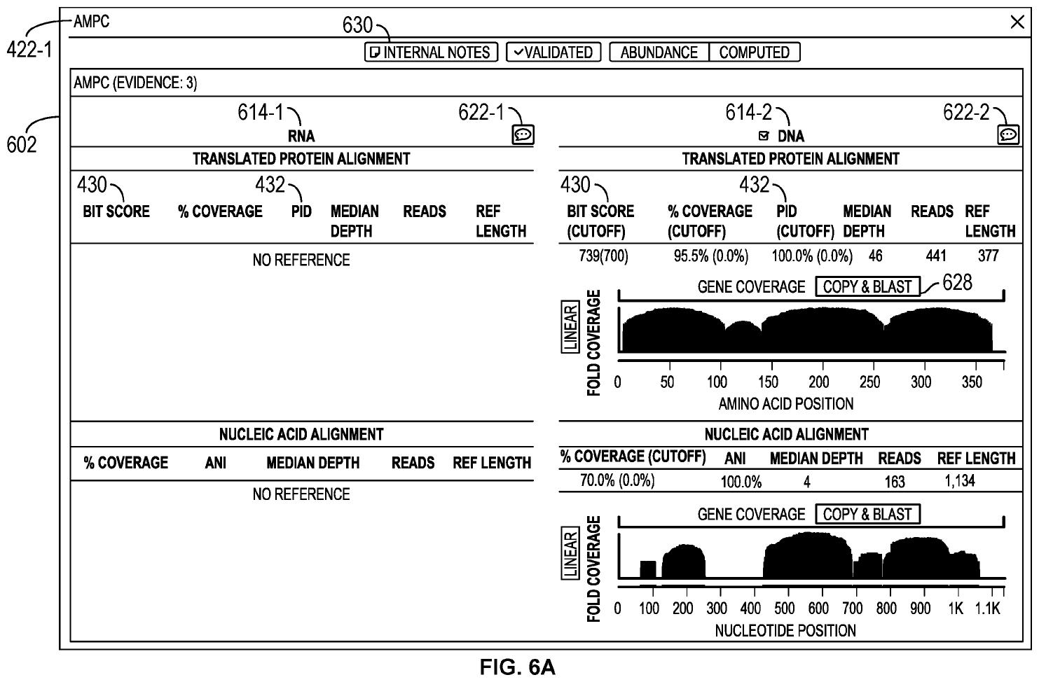

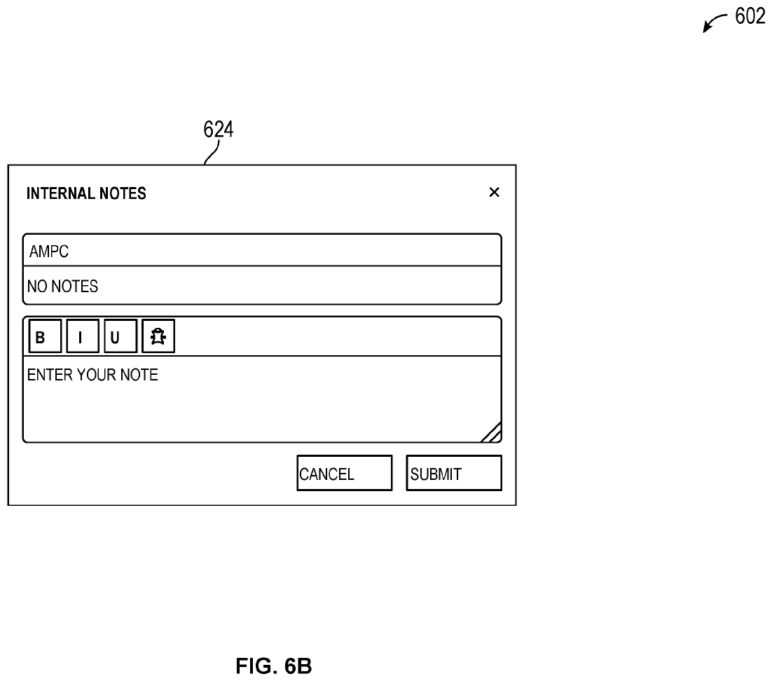

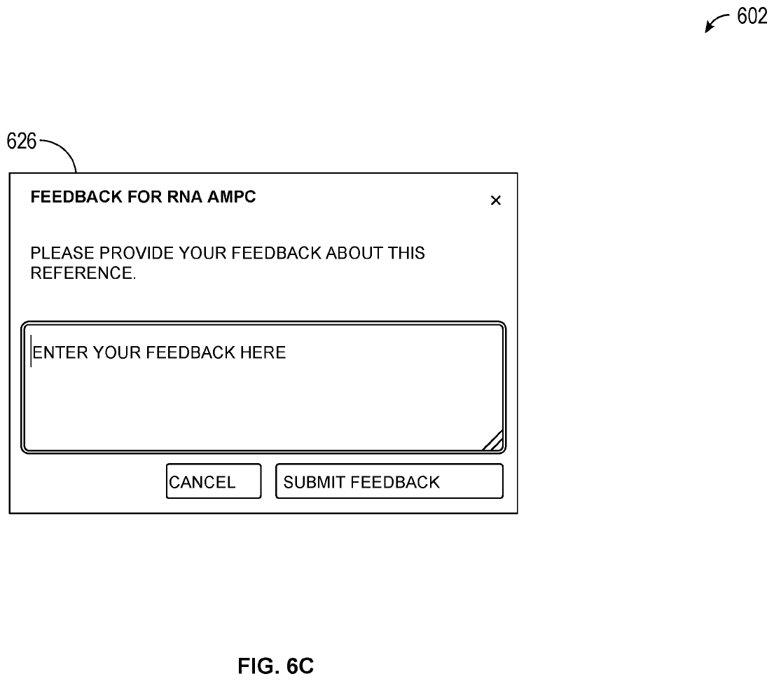

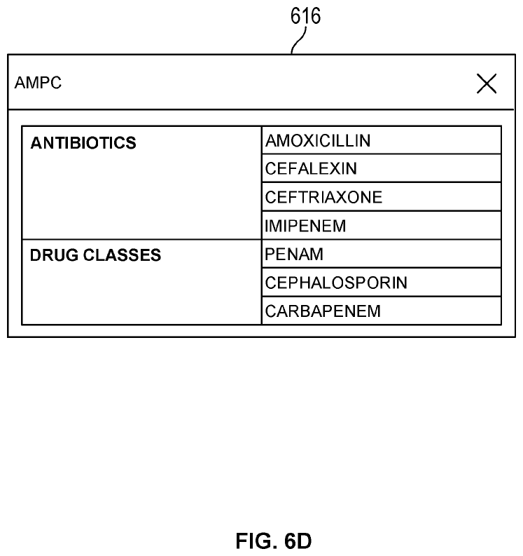

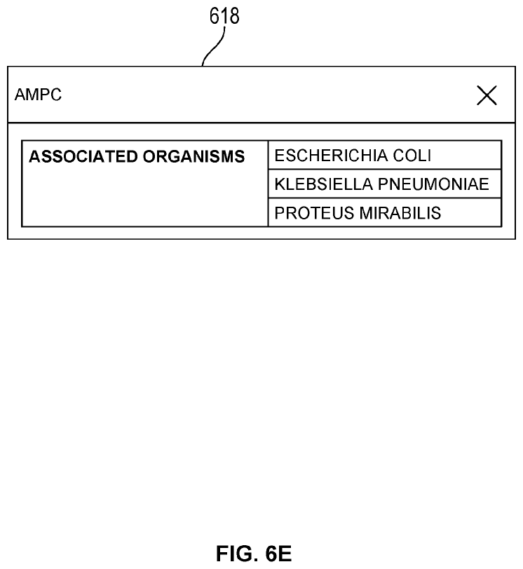

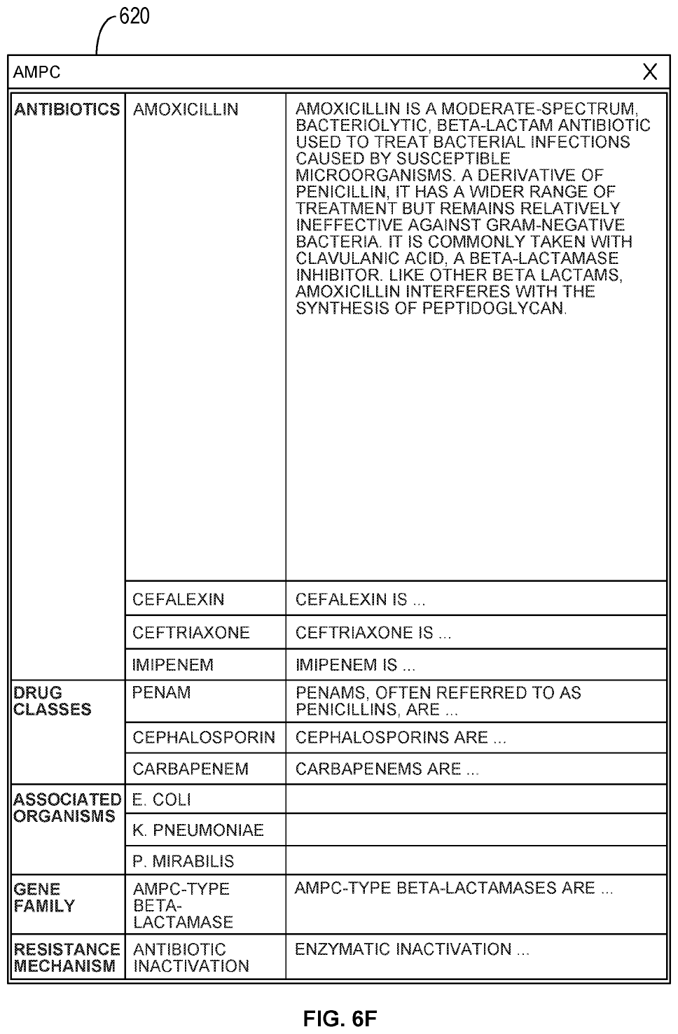

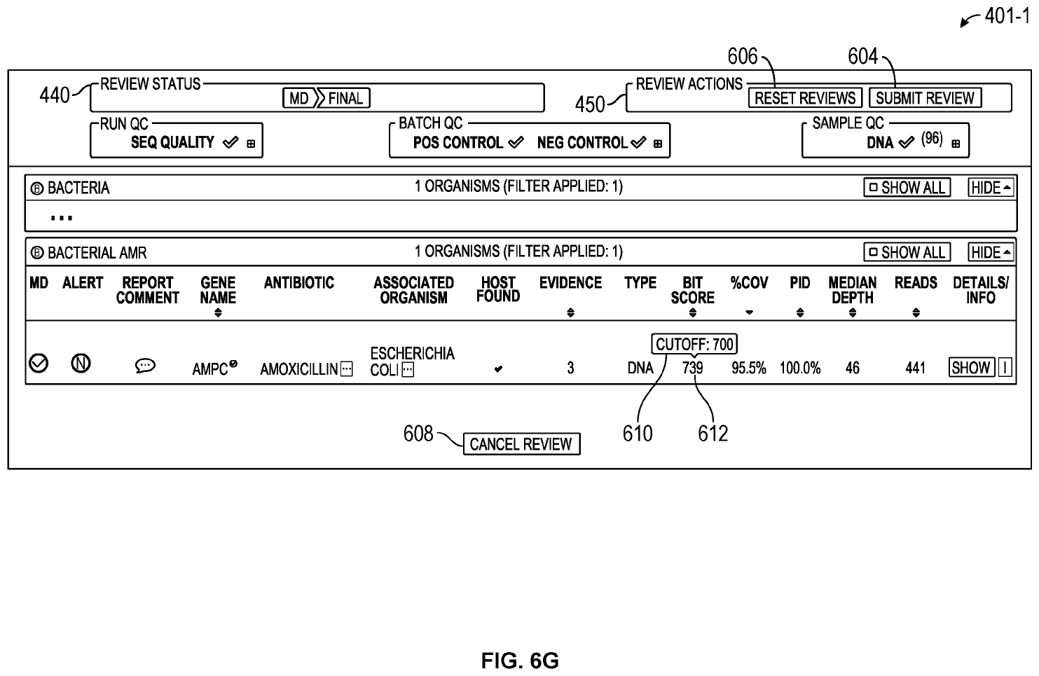

As illustrated in FIGS. 4 and 6A-G, in some embodiments, the customizable user interface further comprises, for each antimicrobial resistance marker (e.g., AMR gene) in the subset of the plurality of mapping statistics, an affordance 436 for displaying one or more features for the respective antimicrobial resistance marker (e.g., gene identifier, gene name, antibiotic information, drug classes, associated organisms, gene families, and/or resistance mechanisms). Example embodiments for displaying one or more features for antimicrobial resistance markers are further described in the section entitled “Viewing features,” below.

Other Metrics.

Referring to FIGS. 3A-B and 4, in some embodiments, the result set further comprises a plurality of additional run, batch, and/or sample-level metrics, including metadata.

For example, in some embodiments, a respective result set 122 for a biological or non-biological sample 304 in an index of biological or non-biological samples for the user (e.g., the results dashboard 302 and/or sample queue 306) further comprises a review status 316, an accession code, a sample name, a sample type (e.g., sample descriptor, a tissue of origin, a type of biopsy sample, etc.), a sample description (e.g., descriptors for sample handling and/or processing), a test profile, a sample summary 318 (e.g., an overview of the run and/or mapping statistics), a run identifier 314, a batch identifier 310. a run directory (e.g., an identifier for a location of a digital result set in a local or cloud-based computing infrastructure), a run completion time, an analysis platform version, a review platform version, a pipeline version, an analysis version, an analysis completion time, an identity of a user, and/or an identity of a reviewing user (e.g., a medical director and/or a final reviewer). In some embodiments, one or more additional metrics are displayed on a visualization system such as results dashboard 302, where selection of the one or more additional metrics for display is performed using an affordance 326. For example, in FIGS. 3A-B. selection of affordance 326 expands a dropdown list 328, from which any of the additional metrics can be selected or deselected for viewing in results dashboard 302.

Other non-limiting examples of run, batch, and/or sample metrics include a review status, a run accession number, a positive control identifier, a negative control identifier, a total number of samples in an index of biological or non-biological samples, a number of samples in a batch, a number of batches in a run, sequencing protocol metrics (e.g., RNAseq, whole transcriptome, panel enriched, and/or shotgun workflows), mapping protocol metrics, positivity rates (e.g., positive hits in patients compared to controls), a reference genome identifier (accession number), a uniqueness (e.g., specificity of an alignment of a nucleotide sequence to a region of a genome), and/or an annotation status (e.g., based on a database, published data, etc.). In some embodiments, additional metrics and/or metadata for a sample 304 is displayed upon receiving a request to display an analysis (e.g., customizable user interface 401-1) of a result set for the respective sample. For example, as illustrated in FIG. 4, in some implementations, additional metrics and/or metadata for the sample are displayed as a header 438 in customizable user interface 401-1.

Quality Control Data.

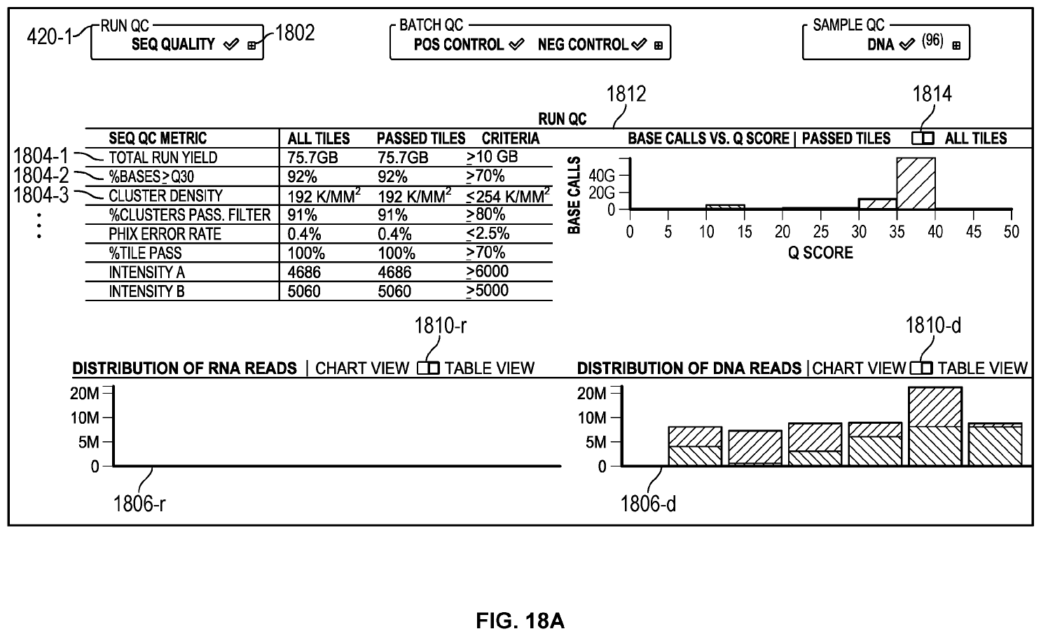

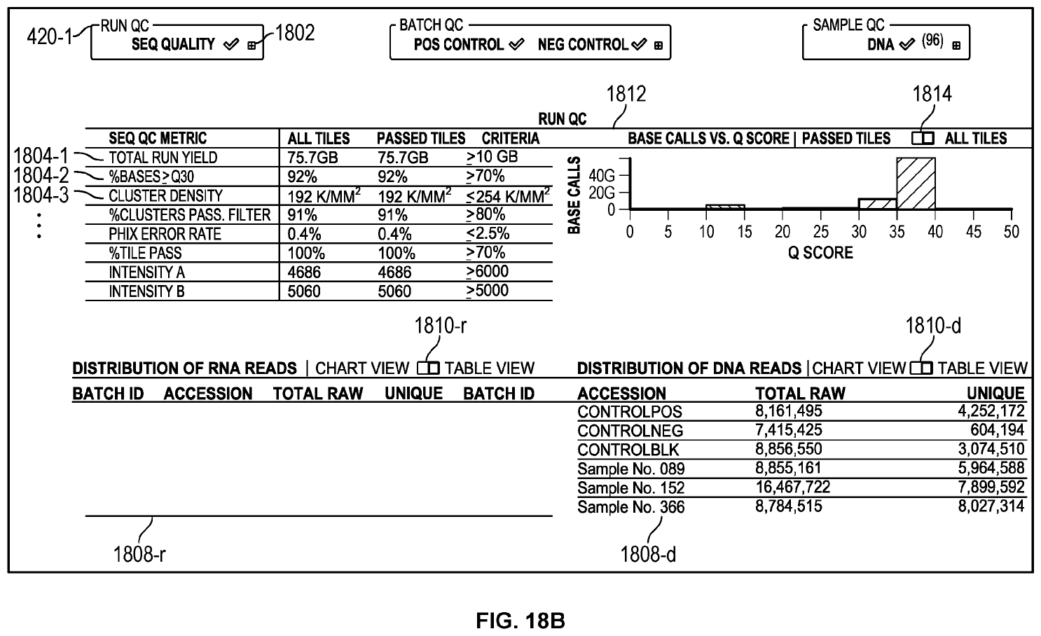

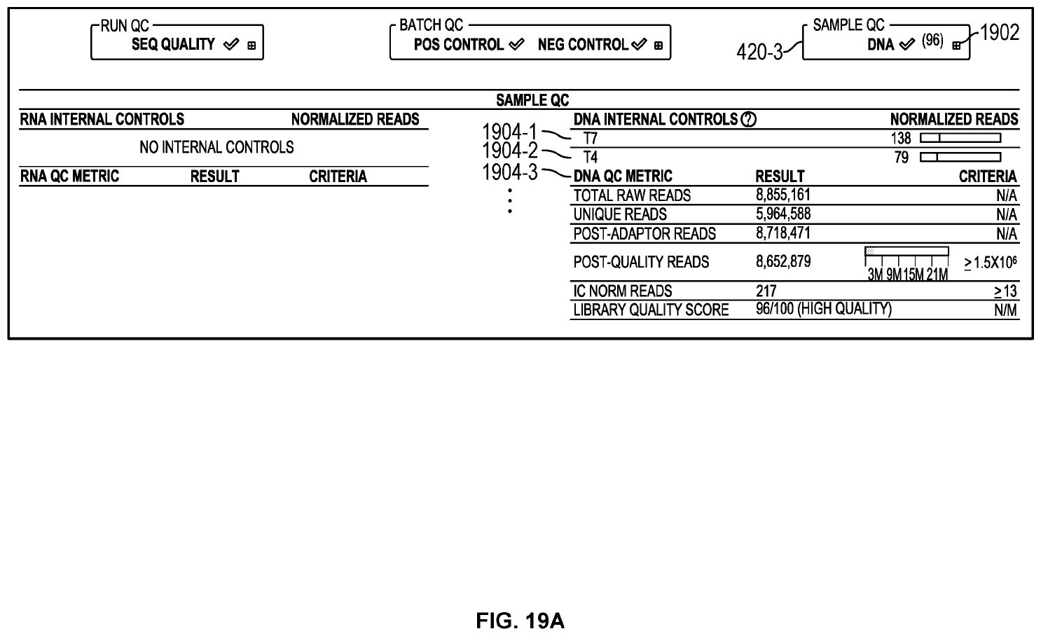

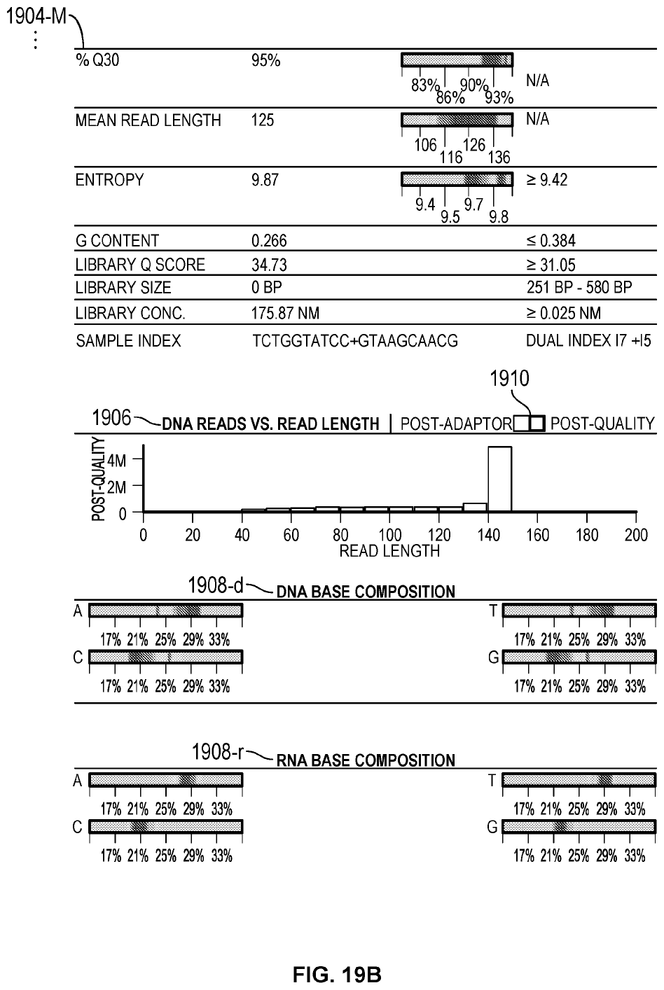

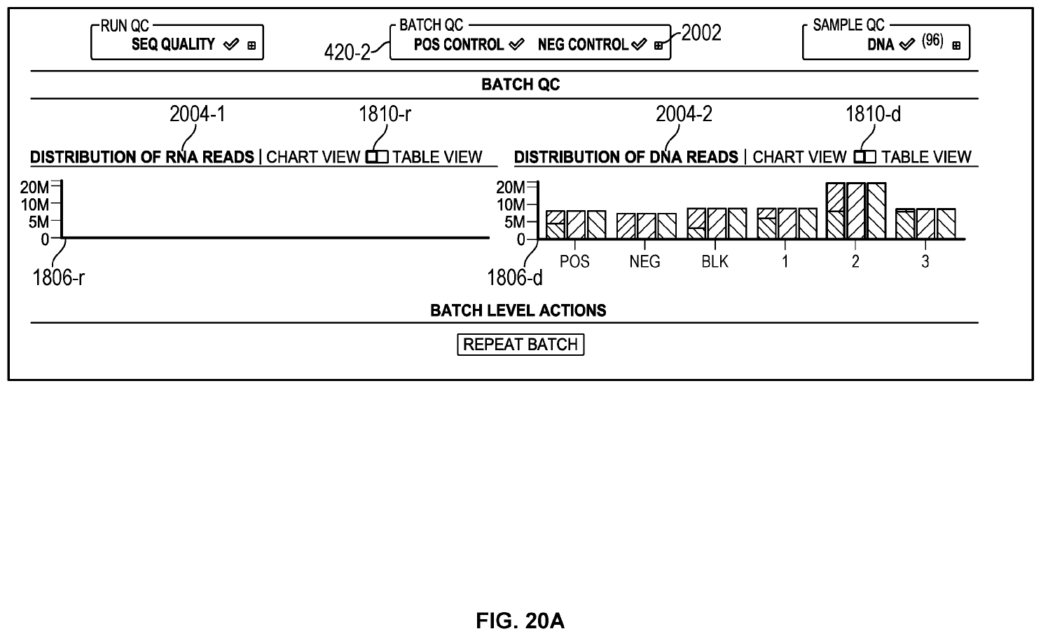

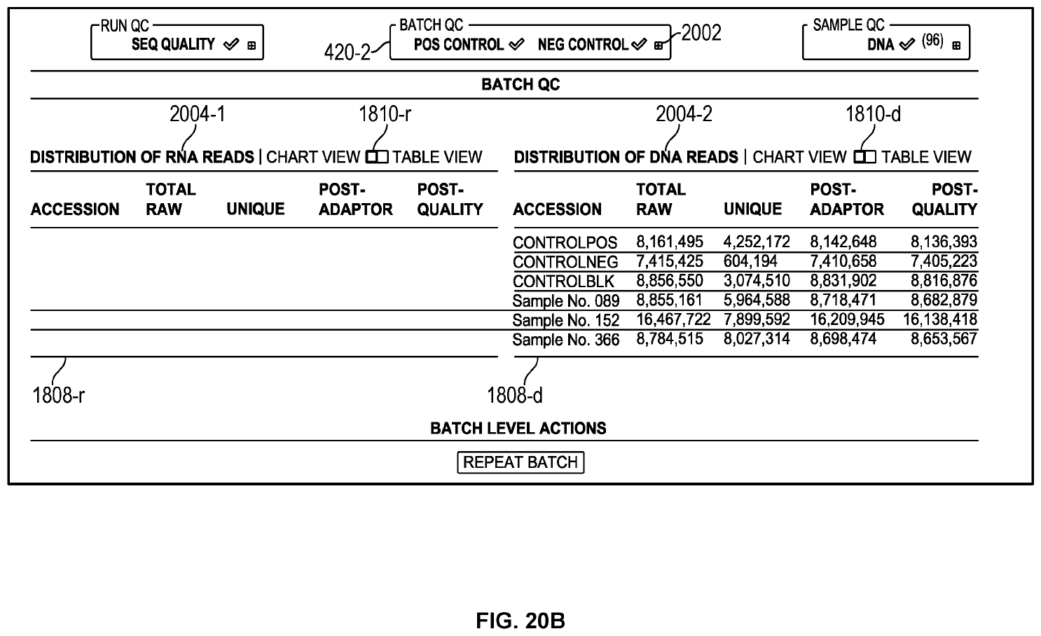

As illustrated in FIGS. 18A-B, 19A-B, and 20A-B, selection of a sample can also include displaying, on the display, a plurality of quality control data, such as sequencing and mapping quality control data. For example, presentation of quality control data allows a user to assess whether a sequencing and/or mapping has been performed successfully before determining whether the output of the analysis is accurate and meaningful. Confirmation that control and analysis samples have passed quality control checks provides assurance that any subsequent analytical results and/or interpretations are reliable at least based on the performance of the sequencing and mapping. In some embodiments, quality control data is displayed on a sample level (e.g., 420-3), a batch level (e.g., 420-2), and/or a run level (e.g., 420-1). In some embodiments, quality control data is displayed upon selection of an affordance. For example, as illustrated in FIGS. 4 and 18A-B, selection of an affordance 1802 for run-level quality control data 420-1 in customizable user interface 401-1 expands a display window that includes quality control data 1804 (e.g., 1804-1, 1804-2, 1804-3, etc.). Similarly, as illustrated in FIGS. 4 and 19A-B, selection of an affordance 1902 for sample-level quality control data 420-3 in customizable user interface 401-1 expands a display window that includes quality control data 1904 (e.g., 1904-1, 1904-2, 1904-3 . . . . , 1904-M, etc.). FIGS. 4 and 20A-B further illustrate selection of an example affordance 2002 for batch-level quality control data 420-2 in customizable user interface 401-1 that expands a display window including quality control data 2004 (e.g., 2004-1, 2004-2, etc.).

For example, non-limiting examples of sequencing and/or mapping quality control metrics (e.g., 1804, 1904, and/or 2004) include an error rate (e.g., a PhiX error rate), a Q score, a fluorescence intensity (e.g., intensity A and/or intensity C), a measure of reagent fluorescence, a cluster density, a Q score passing metric that includes a count (e.g., a percentage) of bases that pass a Q30 threshold value, a filter passing metric that includes a count (e.g., a percentage) of clusters that pass a quality control filter, one or more adapter dimer metrics, internal controls (e.g., for DNA and/or RNA), a count (e.g., a percentage) of sequencing tiles that passed some or all of the quality control checks, and/or a presence or absence of IC failure. In some embodiments, quality control data is displayed for a positive control sample, a negative control sample, a blank control samples, and/or an analysis sample.

Selection and/or visualization of quality control data, in some embodiments, also includes displaying, on the display, the cutoff thresholds for one or more quality control metrics (e.g., criterion or criteria). For example, in some such embodiments, a score meeting and/or exceeding the cutoff threshold for a quality control metric is required to pass a respective quality control check.

In some embodiments, quality control data is displayed as a text-based representation, a graphical representation, and/or a table. For example, referring to FIGS. 18A-B and 20A-B, in some embodiments, the quality control data displayed on the display is viewed as a chart view mode 1806 (e.g., RNA 1806-r and/or DNA 1806-d) and/or a table view mode 1808 (e.g., RNA 1808-r and/or DNA 1808-d). In some embodiments, the selection of the chart view mode and/or the table view mode is operated via a user affordance, e.g., a toggle button. In FIGS. 18A-B and 20A-B, selection of an affordance 1810 (e.g., 1810-r and/or 1810-d) adjusts the representation of the quality control data 1804 from a chart view 1806 to a table view 1808.

In some embodiments, quality control data is plotted as a bar chart. For example, FIGS. 18A and B illustrate a plot 1812 of base calls versus Q score and a plot 1806 of the distribution of DNA reads. FIG. 19B illustrates a plot 1906 of DNA reads versus read length and plots 1908 of DNA and RNA base compositions (e.g., 1908-d and/or 1908-r). Graphical representations of quality control data can be manually adjusted to display data corresponding to all sequencing tiles included in the sequencing reaction (e.g., via a user affordance such as toggle button 1814). Alternatively, graphical representations of quality control data can be manually adjusted to display only data corresponding to sequencing tiles in the sequencing reaction that passed one or more quality control thresholds (e.g., via a user affordance such as toggle button 1814). Other user affordances for switching between alternative views are possible, such as an affordance for applying a pre-processing criterion filter to the plurality of nucleotide sequences used for generating the quality control data (e.g., a post-adaptor/post-quality toggle button 1910). In some embodiments, quality control data for a plurality of samples is aggregated prior to presentation and visualization. In some such embodiments, the quality control data includes batch-level quality control data. Aggregated quality control data (e.g., batch quality control data) can also be presented as a graphical representation.

Features of the Analysis.

Referring again to FIG. 2 at Block 204, the method further comprises applying, responsive to the request (e.g., to display an analysis of the result set 122 obtained from a sequencing reaction of nucleic acids from the biological or non-biological sample 304), a first customizable diagnostic template 138-1 to the result set 122, where the customizable diagnostic template specifies (i) a subset of the plurality of sequencing statistics 128, (ii) a subset of the set of microorganisms, and (iii) a subset of the plurality of mapping statistics 126.

In some embodiments, the request to display the analysis of the result set is afforded by a selection (e.g., a user selection) of a run (e.g., 314) in an index of runs, a batch (e.g., 310) in an index of batches, and/or a sample (e.g., 304) in an index of samples. For example, in some embodiments, the run, batch, and/or sample is selected from an index of runs, batches, and/or samples displayed on a user-interactive results dashboard (e.g., 302). In some embodiments, the method comprises applying, responsive to the request, a first customizable diagnostic template 138-1 to each respective result set 122 corresponding to each respective sample in a batch. In some embodiments, the method comprises applying, responsive to the request, a first customizable diagnostic template 138-1 to each respective result set 122 corresponding to each respective sample in a run group. For example, in some embodiments, the method further comprises a customizable diagnostic template 138-1 that can be applied during batch processing.

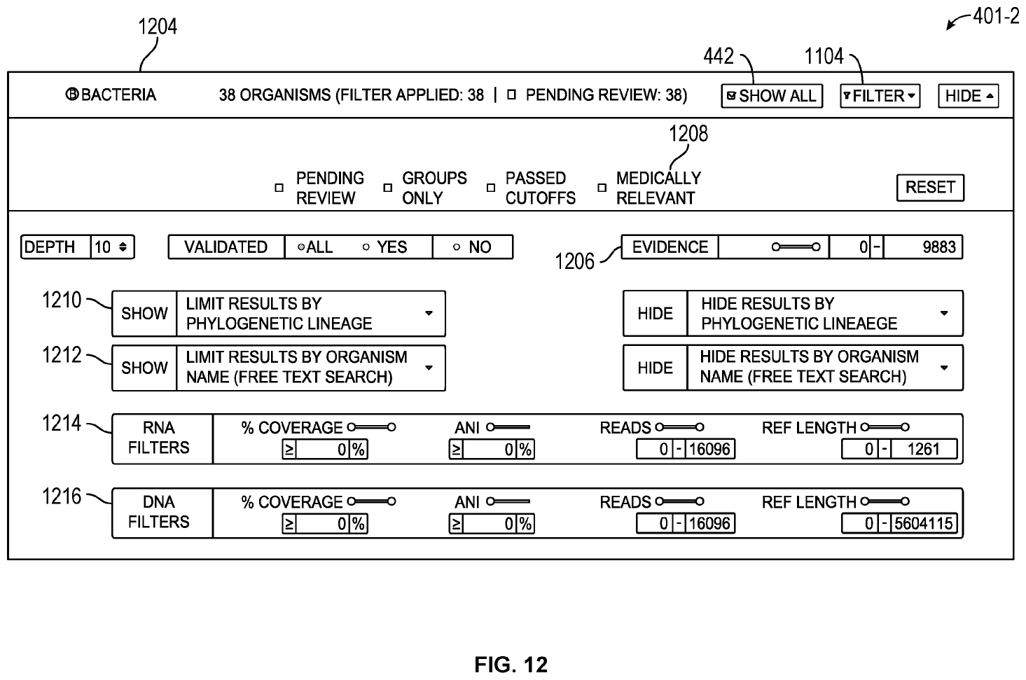

In some embodiments, the specifying the subset of the plurality of sequencing statistics, the subset of the set of microorganisms, and the subset of the plurality of mapping statistics is based on a plurality of parameters that are used as selection criteria applied to the plurality of sequencing statistics, the set of microorganisms, and the plurality of mapping statistics. In some embodiments, the plurality of parameters is predefined (see: Parameters for feature selection, below). In some embodiments, the plurality of parameters is user-specified (see, Customizable analysis of presence of microorganisms. below). Parameters for selection criteria are further illustrated, for example, in FIGS. 12 and 23A-I.

In some embodiments, the applying the first customizable diagnostic template to the result set generates a plurality of features including but not limited to the subset of the plurality of sequencing statistics, the subset of the set of microorganisms, and the subset of the plurality of mapping statistics specified by the first customizable diagnostic template. For example, as will be described in further detail below, in some embodiments the plurality of features includes additional features relating to the viewing, review, visualization, modification, validation, and/or reporting of the analysis of presence of microorganisms.

As used herein, the term “features” refers to any of the information and/or data included in or relating to viewing, review, visualization, modification, validation, and/or reporting of the analysis of presence of microorganisms in the result set. In some embodiments, the plurality of features includes the information and/or data presented in the result set after application of the first customizable diagnostic template. In some such embodiments, the plurality of features includes the subset of the plurality of sequencing statistics, the subset of the set of microorganisms, and/or the subset of the plurality of mapping statistics.

For example, in some embodiments, the subset of the plurality of sequencing statistics includes any one or more sequencing statistics as described herein (see, Sequencing statistics, above), and/or any combination thereof. Similarly, in some embodiments, the subset of the set of microorganisms can include any one or more microorganisms as described herein (see, Microorganisms, above), and/or any combination thereof, and the subset of the plurality of mapping statistics includes any one or more mapping statistics as described herein (see, Mapping statistics, above), and/or any combination thereof. In addition, in some embodiments, the first customizable diagnostic template further specifies a subset of the plurality of additional metrics (e.g., run, batch, and/or sample-level metrics and/or metadata), which can include any one or more of the plurality of additional metrics as described herein (see, Other metrics, above).

In some embodiments, the plurality of features comprises metadata for the result set prior to or after the application of the first customizable diagnostic template, including run metrics, QC metrics, sample metadata, user interaction metadata (e.g., time-stamps, user logs, user history), review status, alert status, pathogen status, annotations. etc.

In some embodiments, features also refer to any predefined or customizable parameters for the analysis of the result set, including predefined or customizable parameters for the customization of the first customizable diagnostic template, predefined or customizable parameters for the customization of the second customizable diagnostic template, predefined or customizable parameters for the presentation of information (e.g., sample information, result set analysis data, detected or putatively detected microorganisms, sequencing statistics, mapping statistics, run metrics, QC metrics, and/or result set metadata), and/or predefined or customizable parameters for performing actions related to the presentation or analysis of the result set, including selecting, viewing, reviewing, visualizing, modifying, validating, and/or reporting any of the abovementioned features, and/or any affordances for performing the same (e.g., via user interaction).

Visual Indicators and Graphical Representations.

In some embodiments, features also refer to any visual indicators displayed, on the display, for the presentation of any of the abovementioned features, including sample information, result set analysis data, detected or putatively detected microorganisms, sequencing statistics, mapping statistics, run metrics, QC metrics, and/or result set metadata.

In some embodiments, visual indicators include affordances for performing actions, including selection, viewing, review, visualization, modification, validation, and/or reporting of any of the abovementioned features. For example, in some embodiments, an affordance is a text-based or graphical hyperlink that opens a new display. In some embodiments, an affordance is a text-based or graphical operator that performs an action (e.g., an analysis of a result set, an application of a filter to a result set, an approval of a review, a generation of a report, a transmission of a generated report, etc.). In some embodiments, an affordance is an adjustable interactive feature, such as a slider bar and/or a scroll bar (e.g., for adjusting a threshold of a detection threshold). In some embodiments, an affordance is a clickable interactive feature, such as a button or a hyperlink. In some embodiments, an affordance is a toggle button, a checkbox, a radio button, and/or a dropdown list.

In some embodiments, visual indicators include graphical representations of any of the abovementioned features.

In some embodiments, visual indicators include text-based representations of any of the abovementioned features.

In some embodiments, a visual indicator is an alphanumeric character, a string of alphanumeric characters, a shape, an image, a color, and/or a pattern.

In some embodiments, visual indicators include a plurality of other metrics and/or metadata, including sequencing statistics, mapping statistics, and/or quality control data that are displayed, on the display, as a text-based or graphical representation, responsive to a detection of a selection of a biological or non-biological sample.

In some embodiments, a graphical representation includes heatmaps, bar graphs, density plots, dot plots, line graph, area graph, scatter plot, box and whisker plot, violin plot, histogram, pie chart, and/or any form of graphical representation as will be apparent to one skilled in the art.

Viewing Features.

Referring to FIG. 2 at Block 206, the method further comprises displaying, on the display, a customizable user interface comprising (i) a review status for the nucleic acid sequencing data, (ii) a first affordance for updating the review status for the nucleic acid sequencing data, (iii) a summary of the subset of the plurality of sequencing statistics 128, (iv) for each respective microorganism in the subset of the set of microorganisms (e.g., the subset of the set of at least 3, at least 5, or at least 10 microorganisms) satisfying a minimum mapping threshold in the result set, a corresponding summary of the subset of the plurality of mapping statistics 126 for the respective nucleotide sequences in the plurality of nucleotide sequences mapped to the reference sequence of the respective microorganism, and (v) a second affordance for applying a second customizable diagnostic template 138-2 to the result set. For example, FIGS. 4, 6G, and 11 illustrate an example customizable user interface 401-1 including (i) a review status for the nucleic acid sequencing data 440, (ii) a first affordance 604 for updating the review status 440 for the nucleic acid sequencing data, (iii) a summary of the subset of the plurality of sequencing statistics 420 (e.g., 420-1, 420-2, 420-3), (iv) for each respective microorganism 402 (e.g., 402-1) in the subset of the set of microorganisms satisfying a minimum mapping threshold in the result set, a corresponding summary of the subset of the plurality of mapping statistics (e.g., 404-418, 424-428), and (v) a second affordance (e.g., affordances 442 and/or 1104) for applying a second customizable diagnostic template 138-2 to the result set 122.

In some embodiments, the customizable user interface comprises any visual indicators and/or text-based or graphical representations as described above to convey information for one or more features of the analysis.

In some embodiments, in addition to displaying features (i) through (v) above, the customizable user interface further includes a corresponding summary of the subset of the plurality of additional metrics (e.g., run, batch, and/or sample-level metrics and/or metadata) specified by the first customizable diagnostic template.

FIG. 4 illustrates an example of a customizable user interface 401-1 that is displayed upon application of a first customizable diagnostic template to the result set for a sample 304-1 (e.g., sample no. 5958).

In some embodiments, the review status 440 for the nucleic acid sequencing data indicates the current review status and the next following review status. For example, in FIG. 4, the current review status 440-1 is marked as “MD” and the next following review status 440-2 (e.g., following submission of the current review) is marked as “Final.” in some embodiments, the review status is selected from the group consisting of first review, second review, medical director (e.g., “MD”) review, final review, passed, and approved.

In some embodiments, as illustrated in FIG. 6G, the first affordance for updating the review status is selected from one or more review actions 450. For example, in some embodiments, selection of an affordance for submitting a review (e.g., Submit Review 604) updates the review status by submitting the current review. In some such embodiments, after submission of the current review (e.g., an MD review), the result set is available to be reviewed by the next reviewer (e.g., a final review). In some embodiments, the customizable user interface further includes an affordance for resetting a review 606 (e.g., to a default state). In some embodiments, the customizable user interface further includes an affordance for cancelling a review 608. In some embodiments, e.g., returning to FIG. 4, selection of the affordance for updating the review status is dependent upon completion of a review, such that the review status cannot be updated while the review is pending (e.g., “Final Review Pending”). In some embodiments, the summary of the subset of the plurality of sequencing statistics 128 includes any of the sequencing statistics disclosed herein.

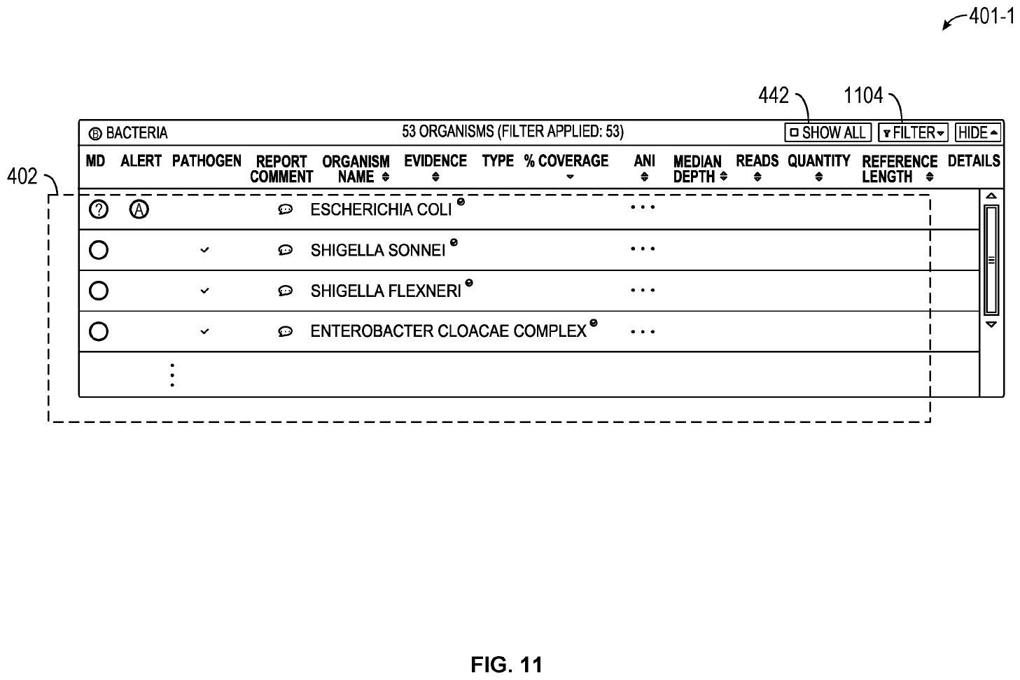

FIGS. 3A-B and 4 further illustrate example summaries of the subset of the plurality of sequencing statistics 128; for instance, in some embodiments, the summary of the subset of sequencing statistics includes a summary of one or more quality control metrics 420 and/or a visual indication of one or more quality control metrics 320 and/or 322. In some embodiments, for each respective microorganism in the subset of the set of microorganisms satisfying a minimum mapping threshold in the result set, a corresponding summary of the subset of the plurality of mapping statistics for the respective nucleotide sequences mapped to the reference sequence of the respective microorganism includes any of the embodiments for mapping statistics 126 disclosed herein (e.g., 404-418, 422-432). In some embodiments, selection of the second affordance for applying a second customizable diagnostic template 138-2 to the result set modifies one or more of (i) the subset of the plurality of sequencing statistics 128, (ii) the subset of the set of microorganisms, and (iii) for each respective microorganism in the subset of the set of microorganisms, the corresponding subset of the plurality of mapping statistics 126. For example, as illustrated in FIGS. 4 and 11, an example second affordance 442 (e.g., “Show All”) expands the customizable user interface 401-1 to display an expanded subset of the set of microorganisms. In some embodiments, selection of the second affordance 442 (e.g., “Show All”) expands the customizable user interface 401-1 to display all of the microorganisms in the set of microorganisms. In other embodiments, as illustrated in FIGS. 11 and 12, an example second affordance 1104 (e.g., “Filter”) displays one or more filters that are applied to the (i) the subset of the plurality of sequencing statistics 128, (ii) the subset of the set of microorganisms, and/or (iii) for each respective microorganism in the subset of the set of microorganisms, the corresponding subset of the plurality of mapping statistics 126. For example, in some implementations, selection of the second affordance 1104 applies the second customizable diagnostic template 138-2 to the result set, where the second customizable diagnostic template includes one or more parameters 1204 for filtering the sequencing statistics, microorganisms, and/or mapping statistics for display. Application of the second customizable template, including filters and parameters. are further described herein, such as in the sections entitled “Filters” and “Parameters for feature selection,” below.

In some embodiments, the customizable user interface further comprises a count of microorganisms detected in the biological or non-biological sample 304. In some embodiments, the customizable user interface further comprises an identity of each microorganism 402 detected in the biological or non-biological sample 304. In some embodiments, the customizable user interface further comprises an identity of an AMR gene 422 detected in the biological or non-biological sample 304.

For example, FIG. 4 illustrates a display including a count of microorganisms detected in a sample 304-1 (e.g., 1 organism), an identity of a bacterial microorganism (e.g., Escherichia coli 402-1), and a bacterial antimicrobial resistance gene (e.g., ampC 422-1) detected in the sample.

In some embodiments, the subset of the set of microorganisms satisfying the minimum mapping threshold is a threshold number of microorganisms with the highest values for a percent sequence alignment, based on an alignment of respective nucleotide sequences to the reference sequence of the respective microorganism. For example, in some embodiments, the subset of the set of microorganisms (e.g., the subset of the set of at least 3, at least 5, or at least 10 microorganisms) is the top N microorganisms with the highest percent sequence alignment. In some such embodiments, N is a positive integer. In some embodiments, Nis 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30.

In some embodiments, the subset of the set of microorganisms satisfying the minimum mapping threshold is a threshold number of microorganisms with the highest values for a sequencing coverage, based on the mapping of respective nucleotide sequences to the reference sequence of the respective microorganism. For example, in some embodiments, the subset of the set of microorganisms is the top N microorganisms with the highest sequencing coverage. In some such embodiments, N is a positive integer. In some embodiments, N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30.

In some embodiments, the minimum mapping threshold is determined based on a minimum confidence score obtained using at least a coverage, a uniqueness metric, and an annotation metric for each respective nucleotide sequence in the plurality of nucleotide sequences that maps to the reference sequence for the respective microorganism.

In some embodiments, the minimum mapping threshold is user-customizable. In some embodiments, the minimum mapping threshold is predefined.

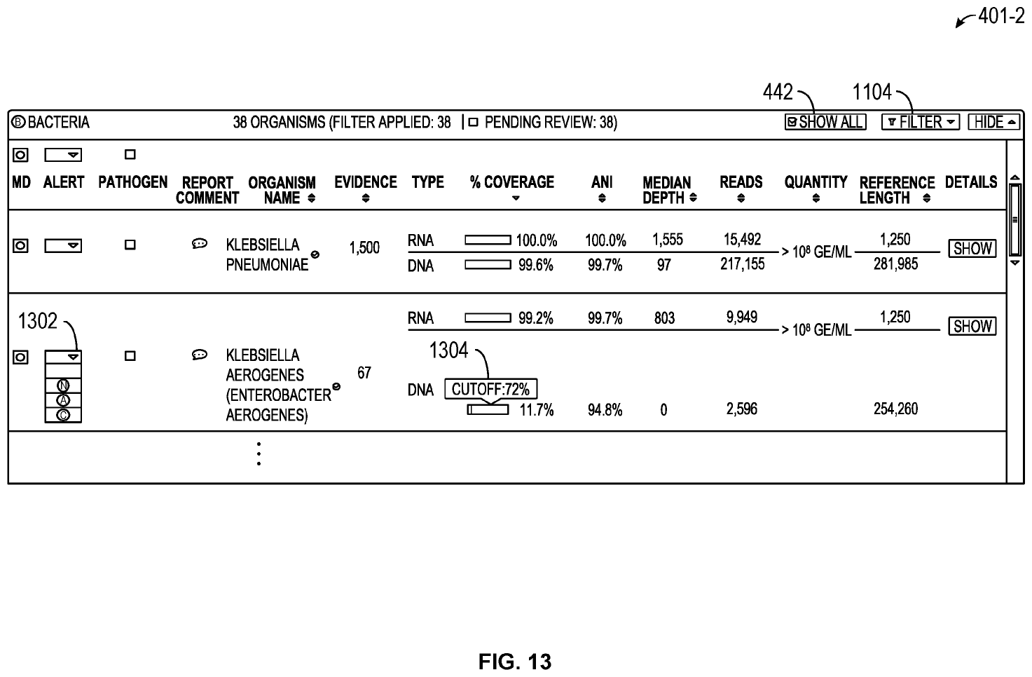

In some embodiments, a user interaction is used to view and/or display one or more features in the customizable user interface 401. In some embodiments, a user interaction includes clicking on a feature (e.g., an organism name) to view expanded feature information. In some embodiments, a user interaction includes hovering a pointer (e.g., a mouse) over a feature to view expanded feature information. For example, FIG. 6G illustrates an example of a user interaction for displaying an expanded feature information in a customizable user interface 401-1. As illustrated in FIG. 6G, hovering a pointer over a mapping statistic marker (e.g., a bit score) displays an overlay display 610 of the cutoff threshold for determining whether the respective feature (e.g., an antimicrobial resistance (AMR) gene) is detected in the sample and thus whether it will be displayed in the customizable user interface. For instance, for a predefined or user-specified bit score cutoff threshold (e.g., 700), in some embodiments, an AMR gene is displayed when the bit score of the AMR gene 612 exceeds the cutoff threshold (e.g., 739). As an alternate example, FIG. 13 illustrates a similar user interaction for displaying an expanded feature information. As shown in FIG. 13, hovering a pointer over a percent coverage mapping statistic displays an overlay display 1304 of the cutoff threshold for detection for percent coverage (e.g., 72%). Note that in this instance, the percent coverage mapping statistic for the respective microorganism Kiebsiella aerogenes fails to satisfy the cutoff threshold for detection, and thus the respective microorganism and its corresponding mapping statistics are not displayed upon application of the first customizable diagnostic template.

In some embodiments, the viewing expanded feature information generates a new display (e.g., a new window, a new tab, or an overlay display such as a popup window). In some embodiments, the new display has an affordance for canceling the new display of the expanded feature information (e.g., a close-out or exit button, a back button, etc.). In some embodiments, the new display of the expanded feature information is canceled by user interaction (e.g., clicking a mouse) on a portion of the display that does not contain the expanded feature information (e.g., for an overlay display or popup window, the display of the expanded feature information can be canceled by clicking anywhere on the screen outside of the popup window).

In some embodiments, the viewing expanded feature information is displayed as a transitory display where visibility is dependent on instant or present user interaction. For example, in some embodiments, the expanded feature information is presented as an overlay only when a user directs a pointer (e.g., a mouse) to a specific location on the display. When the user moves the pointer to a different location on the display, the overlay is removed. For example, as shown in FIGS. 6G and 13, hovering a mouse over a statistic marker displays an overlay display (e.g., 610, 1304) of a detection threshold, which is removed when the pointer is moved away from the respective marker.

In some embodiments, upon receiving a user interaction, the display displays a change in a visual indicator. For example, where a visual indicator is an alphanumeric character, a string of alphanumeric characters, a shape, an image, a color, and/or a pattern, a change in a visual indicator can include a change in the alphanumeric character, the string of alphanumeric characters, the shape, the image, the color, and/or the pattern. In some such embodiments, the change in the visual indicator includes a change in the intensity, size, thickness, and/or formatting of any of the above visual indicators. In some embodiments, the change in a visual indicator upon receiving a user interaction includes displaying a visual indicator where a visual indicator was not previously displayed.

Referring to FIG. 2 at Block 208, in some embodiments, the customizable user interface further comprises (vi) a third affordance for expanding upon the corresponding summary of the subset of the plurality of sequencing statistics, and (vii) a fourth affordance for expanding upon a summary of a plurality of values for the subset of the plurality of mapping statistics.

In some embodiments, as illustrated in FIGS. 4, 18A-B, 19A-B, and 20A-B, the corresponding summary of the subset of the plurality of sequencing statistics includes one or more quality control metrics, and the third affordance (e.g., 1802, 1902, and/or 2002) expands the display accordingly. In some embodiments, upon user selection of the third affordance, the method further comprises displaying, on the display, a graphical representation of a sequencing statistic in the subset of the plurality of sequencing statistics. In some embodiments, expanding the summary of the subset of the plurality of sequencing statistics displays the subset of the plurality of sequencing statistics. In some embodiments, the display is provided in a new display window (e.g., a popup window). In some such embodiments, the displaying the subset of the plurality of sequencing statistics displays one or more visual indicators and/or text-based or graphical representations for each sequencing statistic in the subset of the plurality of sequencing statistics. In some embodiments, the graphical representation is in the form of a heatmap.

In some embodiments, upon user selection of the fourth affordance, the method further comprises displaying, on the display, a graphical representation of a mapping statistic in the subset of the plurality of mapping statistics. In some embodiments, the expanding the summary of the subset of the plurality of mapping statistics displays the subset of the plurality of mapping statistics. In some embodiments, the display is provided in a new display window (e.g., a popup window). For example, selection of an example fourth affordance 434 (“Show”) illustrated in FIG. 4 provides a new display window 502 for the microorganism 402-1. In some embodiments, the displaying the subset of the plurality of mapping statistics displays one or more visual indicators and/or text-based or graphical representations for each mapping statistic in the subset of the plurality of mapping statistics. In some embodiments, the graphical representation is in the form of a heatmap. In some embodiments, the graphical representation is in the form of a bar graph. In some embodiments, the graphical representation can be viewed in either linear or log scale.

In some embodiments, user selection of the fourth affordance for expanding upon a summary of a plurality of values for the subset of the plurality of mapping statistics comprises selecting a respective microorganism in the subset of the set of microorganisms that satisfies a minimum mapping threshold in the result set.

In some embodiments, each microorganism in the subset of the set of microorganisms that satisfies a minimum mapping threshold in the result set that is displayed in the customizable user interface can be selected by a user, thereby expanding upon the summary of the subset of the plurality of mapping statistics for the respective microorganism.

See, for example, FIGS. 5A and 5B, which illustrate an example of a graphical representation of a mapping statistic (e.g., fold coverage versus nucleotide position) for an alignment of RNA (left panels 504-1) and DNA (right panels 504-2) nucleotide sequences to the genome of a microorganism. The popup display window 502 is overlaid on the customizable user interface 401-1, responsive to a user interaction (e.g., clicking a pointer on the microorganism name). Graphical representations can be toggled between linear scale (FIG. 5A) and log scale (FIG. 5B) via a user interaction (e.g., clicking a pointer) with the visual indicator “Linear” (e.g., 506-A-1, 506-A-2) and/or “Log” (e.g., 506-B-1, 506-B-2). The expanded summary of the subset of the plurality of mapping statistics further includes additional mapping statistics, including percent coverage 408, percent coverage cutoff 409, average nucleotide identity (ANI) 410, number of reads 416, and length of reference genome 418.

In some embodiments, expanding the summary of the subset of the plurality of sequencing statistics (e.g., via user selection of the third affordance) and/or expanding the summary of the subset of the plurality of mapping statistics (e.g., via user selection of the fourth affordance) further comprises displaying a comment appended to a sequencing statistic and/or a mapping statistic. For instance, in some embodiments, as illustrated in FIGS. 5A, 5C, and 5D, the customizable user interface includes an affordance 508 (e.g., 508-1, 508-2) that, upon selection or user interaction, displays a comment window 510 for a respective microorganism. In some embodiments, the comment window further includes an affordance for adding, editing, submitting and/or removing a comment for the respective microorganism. In some embodiments, as illustrated in FIGS. 5C and 5D, the comment is appended to a metric specific to a nucleic acid type (e.g., DNA 510-1 and/or RNA 510-2). In some embodiments, the comment is appended to a summary of a microorganism but is not specific to a nucleic acid type. In some embodiments, the customizable user interface further includes one or more affordances for displaying, adding. modifying, submitting, and/or removing one or more internal notes associated with a respective microorganism. In some embodiments, the customizable user interface further includes one or more affordances for displaying and/or modifying one or more of a validation status, an abundance status, and a computation status associated with a respective microorganism.

In some embodiments, the expanding the summary of the subset of the plurality of sequencing statistics (e.g., via user selection of the third affordance) and/or the expanding the summary of the subset of the plurality of mapping statistics (e.g., via user selection of the fourth affordance) further comprises displaying expanded feature information for an antimicrobial resistance marker 422 (e.g., an AMR gene). For instance, selection of an example affordance 436 (“Show”) illustrated in FIG. 4 provides a new display window 602 for the antimicrobial resistance marker ampC 422-1. As shown in FIG. 6A, in some embodiments, the expanded feature information in new display window 602 for the AMR gene 422-1 includes a text-based and/or a graphical representation (e.g., in togglable linear and/or log scale) of a mapping statistic for the gene. In some embodiments, the representation of one or more mapping statistics (e.g., bit score 430, PID 432, fold coverage versus nucleotide position, etc.) is displayed for an alignment of RNA (left panels 614-1) and DNA (right panels 614-2) nucleotide sequences to the sequence of the antimicrobial resistance marker in the reference genome of the microorganism. In some such implementations, the representation indicates the alignment of translated protein and/or nucleic acids. As shown in FIG. 6D, in some embodiments, the expanded feature information for the AMR gene includes an annotation 616 of the gene with one or more of a therapeutic treatment and a drug class associated with the gene (e.g., displayed upon selection of an antibiotic affordance 444). As shown in FIG. 6E, in some embodiments, the expanded feature information for the AMR gene includes an annotation 618 of the AMR gene with a microorganism associated with the gene (e.g., displayed upon selection of an associated organism affordance 446). As shown in FIG. 6F, in some embodiments, the expanded feature information for the AMR gene includes an annotation of the gene with aggregated information 620 (e.g., antibiotics information, drug class information, associated organisms, gene family, and/or resistance mechanisms) associated with the gene (e.g., displayed upon selection of an information affordance 448). As shown in FIGS. 6B and 6C, in some embodiments, the displaying expanded feature information for an AMR gene 422-1 in new display window 602 includes displaying a comment appended to the gene. For instance, in some embodiments, the display window 602 includes an affordance 622 (e.g., 622-1, 622-2) that, upon selection or user interaction, displays a comment window (e.g., 626) for a respective antimicrobial resistance marker. In some embodiments, the comment window further includes an affordance for adding, editing. submitting and/or removing a comment for the respective antimicrobial resistance marker. In some embodiments, the comment is appended to a metric specific to a nucleic acid type (e.g., DNA and/or RNA 626). In some embodiments, the comment is appended to a summary of the AMR gene but is not specific to a nucleic acid type. In some embodiments, the display window 602 further includes one or more affordances 630 for displaying, adding, modifying, submitting, and/or removing one or more internal notes 624 associated with a respective antimicrobial resistance marker. In some embodiments, the display window 602 further includes one or more affordances for displaying and/or modifying one or more of a validation status, an abundance status, and a computation status associated with a respective antimicrobial resistance marker.

In some embodiments, the customizable user interface further comprises a summary of a subset of a plurality of sequencing quality control metrics, an affordance for expanding upon the corresponding summary of the subset of the plurality of sequencing quality control metrics, a summary of a subset of a plurality of mapping quality control metrics, an affordance for expanding upon the corresponding summary of the subset of the plurality of mapping quality control metrics, a summary of a subset of a plurality of run quality control metrics, and/or an affordance for expanding upon the corresponding summary of the subset of the plurality of run quality control metrics.

In some embodiments, the customizable user interface further comprises a summary of a subset of a plurality of sample-level quality control metrics, an affordance for expanding upon the corresponding summary of the subset of the plurality of sample-level quality control metrics, a summary of a subset of a plurality of batch-level quality control metrics, an affordance for expanding upon the corresponding summary of the subset of the plurality of batch-level quality control metrics, a summary of a subset of a plurality of run-level quality control metrics, and/or an affordance for expanding upon the corresponding summary of the subset of the plurality of run-level quality control metrics. See, for example, FIGS. 18A-B, 19A-B, and 20A-B. In some such embodiments, the affordance for expanding upon the respective summary of the subset of the plurality of quality control metrics is a binary affordance (e.g., a “plus” for expansion, a “minus” for minimization).

Parameters for Feature Selection.

In some embodiments, the first customizable diagnostic template comprises a plurality of parameters 140 for specifying and subsequently displaying, on the customizable user interface, (i) the subset of the plurality of sequencing statistics, (ii) the subset of the set of microorganisms, and (iii) the subset of the plurality of mapping statistics. In some embodiments, selection and display of the subset of the set of microorganisms represents at least a preliminary determination of a presence of the subset of microorganisms in the biological or non-biological sample. Therefore, to ensure accurate determination of the presence of microorganisms, in accordance with some embodiments of the present disclosure, the selection of parameters for applying the first customizable diagnostic template to the result set can be optimized as well. In some embodiments, one or more parameters is selected to specify a minimum mapping threshold in the result set for the respective nucleotide sequences in the plurality of nucleotide sequences that map to the corresponding reference sequence of one or more respective microorganisms in the set of microorganisms. Minimum mapping thresholds are further disclosed herein (see, for example, the section entitled “Viewing features,” above).

Non-limiting examples of parameters 140 used, in some embodiments, for applying the first customizable diagnostic template to the result set include any of the sequencing statistics, mapping statistics, additional metrics, quality control metrics, and/or additional features as disclosed herein and/or as illustrated in FIG. 12 (e.g., adjustable filter cutoffs 1204) and FIGS. 23D (e.g., adjustable mapping statistic cutoffs 2304), 23F (e.g., adjustable cutoffs for run-level sequencing statistics 128), 23G-H (e.g., adjustable cutoffs for sample-level sequencing statistics 128), and 23I (e.g., defined parameters for subclasses 2306 and/or evidence categories 2308). For instance, parameters can be adjusted for any metric, statistic, description, and/or metadata, as disclosed herein. Other parameters associated with, for example, sample metadata, nucleic acid type, sequencing data, sequencing protocol metadata, alignment data, alignment metadata, microorganisms, disease conditions, patient demographics, cohort data, study data, clinical annotations, research reports, therapeutic treatments, and/or antimicrobial resistance are possible, as well as any substitutions, modifications, additions, and/or combinations thereof as will be apparent to one skilled in the art.

In some embodiments, the values of the parameters of the first customizable diagnostic template are predefined (e.g., automated). In some embodiments, the values of the parameters of the first customizable diagnostic template are user-specified (e.g., customizable). Customization of parameters (e.g., for feature selection and determination of presence of microorganisms) is described in detail in a following section (see, Customizable analysis of presence of microorganisms).

In some embodiments, a value of a parameter is a percentage value (e.g., a numeric value between 0 and 100). For example a cutoff threshold for a parameter (e.g., a coverage, an average nucleotide identity, an RNA sensitivity, an RNA specificity, a DNA sensitivity, a DNA specificity, etc.) is between 0 and 10%, between 10 and 20%, between 20 and 30%, between 30 and 40%, between 40 and 50%, between 50 and 60%, between 60 and 70%, between 70 and 80%, between 80 and 90% or between 90 and 100%. In some embodiments, the cutoff threshold for a parameter is at least 70%, at least 80%, at least 90%, at least 95%, at least 96%, at least 97%, at least 98%, or at least 99%/o.

In some embodiments, a value of a parameter is a binary status (e.g., a presence or an absence of a status and/or classification). For example, in some embodiments, a status is pathogen/not pathogen, medically relevant/not medically relevant, validated/not validated (e.g., a review status), and/or quality control check pass/fail.

In some embodiments, a parameter is selected from one or more finite classifications and/or annotations (e.g., microorganism classification (B, F, V, and/or P), etc.).

In some embodiments, a parameter is a keyword or alphanumeric string (e.g., a disease annotation, an organism name, and/or a phylogenetic lineage). In some embodiments, the parameter is a predefined keyword or alphanumeric string that is selected from a finite list of options (e.g., using a dropdown list and/or a checkbox).

In some embodiments, the parameter is a keyword or alphanumeric string that is specified using a free-text search (e.g., via a manual entry box).

In some embodiments, a value of a parameter is a minimum amount of evidence, where evidence is defined as a publication (e.g., in a medical journal, academic journal, and/or conference abstract), annotation (e.g., in a genome database), and/or co-occurrence of a microorganism with a feature of interest, such as a disease condition. In some such embodiments, the value of the parameter is between 0 and 100,000, between 0 and 50,000, between 0 and 20,000, or between 0 and 10.000.

In some embodiments, a parameter is an annotation (e.g., of a microorganism with a disease condition and/or other clinical or diagnostic feature of interest). For example, in some embodiments, a respective microorganism is annotated with an annotation if a co-occurrence of the microorganism and the feature of interest is observed at least a minimum number of times in, e.g., clinical or academic literature, pathogen databases, and/or other resources such as a digital library or a nucleic acid database.

Additionally, in some embodiments, the value of a parameter is any value for a respective feature that is known in the art to be standard or substantially standard for reliable sample processing and analysis, such as passing a quality control check. In some embodiments, the value of a parameter is empirically determined (e.g., based on laboratory experimentation). In some embodiments, the value of a parameter is optimized for detection of a specific microorganism, disease condition, and/or antimicrobial resistance marker of interest.

Other non-limiting parameters for feature selection include depth, read count, and/or reference length. For example, in some embodiments, the cutoff threshold for depth is at least 1, at least 2, at least 5, at least 10, at least 20, at least 100, at least 200, at least 500, or at least 1,000.

In some embodiments, the cutoff threshold for RNA read count is between 0 and 10 million, between 0 and 5 million, between 0 and 1 million, between 0 and 750,000, between 0 and 500,000, between 0 and 200,000, between 0 and 100,000 between 0 and 50,000, or between 0 and 20,000.

In some embodiments, the cutoff threshold for DNA read count is between 0 and 1 billion, between 0 and 100 million, between 0 and 10 million, between 0 and 5 million, between 0 and 1 million, between 0 and 750,000, between 0 and 500,000, or between 0 and 300,000.

In some embodiments, the cutoff threshold for RNA reference length is between 0 and 1 million, between 0 and 500,000, between 0 and 100,000, between 0 and 50,000, between 0 and 10,000 between 0 and 7000, between 0 and 5000, between 0 and 2,000, or between 0 and 1500.

In some embodiments, the cutoff threshold for DNA reference length is between 0 and 1 billion, between 0 and 100 million, between 0 and 10 million, or between 0 and 5 million.

Additional parameters and example ranges for the same are illustrated in FIGS. 23D, 23F, 23G, 23H, and 23I. For example, in some embodiments, a parameter is defined by specifying a minimum or maximum threshold for the respective parameter.

External Links to Databases.

In some embodiments, upon user selection of the fourth affordance, the method further comprises displaying, on the display, an affordance (e.g., 512 and/or 628; see FIGS. 5A and 6A) for accessing a reference sequence database (e.g., a genome database) comprising at least the reference sequence (e.g., genome) for the respective microorganism.

In some such embodiments, additional information for one or more features are accessible through external links, including sequences of reference sequences (e.g., BLAST, NCBI) and/or databases for detected or otherwise selected microorganisms (e.g., EMBL-EBI, GenBank, Ensembl, EuPathDB, The Human Microbiome Project, Pathogen Portal, RDP, SILVA, GREENGENES, EBI Metagenomics. EcoCyc, PATRIC. TBDB, PlasmoDB, the Microbial Genome Database (MBGD), and/or the Microbial Rosetta Stone Database). See, for example, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19: each of which is hereby incorporated by reference herein in its entirety, for additional databases that can be used for analyzing microorganisms.

In some embodiments, a selection of the affordance for accessing a reference sequence database transmits a nucleotide sequence (e.g., the sequence of a reference genome) corresponding to the respective microorganism to the reference sequence database. In some embodiments, the selection of the affordance for accessing a reference sequence database populates an affordance for a manual entry of a text string (e.g., a search box) with the nucleotide sequence (e.g., the sequence of a reference genome) corresponding to the respective microorganism.

Modes of Use.

The review and visualization tools disclosed herein include a plurality of different metrics that provide a user (e.g., a laboratory or medical practitioner) with a comprehensive suite of results in an accessible, streamlined format (e.g., sequencing validation, sequencing statistics, mapping validation, mapping statistics, microorganism detection, microbe-specific annotations, pathogen information, antimicrobial resistance gene expression, and therapeutic treatments, among others). As discussed above, such features allow the analysis and interpretation of nucleic acid sequencing data by users without advanced skills in each and every one of the various aspects of the analysis. In some embodiments, the provided review and visualization tools present a summary of the information relevant to analyzing the presence of microorganisms in a respective biological or non-biological sample such that it can be efficiently examined, understood, and/or reviewed by a practitioner. Further customization is also possible for situations that necessitate fine-tuning.

Automated Analysis of Presence of Microorganisms.

In some embodiments, detection of microorganisms is performed using an automated process, using predefined (e.g., default) thresholds for a plurality of parameters (see, Parameters for feature selection, above). However, these thresholds can be adjusted by a user or practitioner, as discussed in the following sections.

Customizable Analysis of Presence of Microorganisms.

In particular, in addition to an automated process for analysis of the presence of microorganisms, in some embodiments, any one of the parameters and/or detection thresholds can be adjusted based on user preference and/or a priori knowledge. In some embodiments, customizing occurs through a user interaction with one or more affordances.

For example, in some embodiments, an affordance is a text-based or graphical hyperlink that generates a new display (e.g., affordance 434). In some embodiments, an affordance is a text-based or graphical operator that performs an action (e.g., an analysis of a result set, an application of a filter to a result set, an approval of a review, a generation of a report, and/or a transmission of a generated report). In some embodiments, an affordance is an adjustable interactive feature, such as a slider bar and/or a scroll bar (e.g., for adjusting a threshold of a detection threshold). In some embodiments, an affordance is a clickable interactive feature, such as a button or a hyperlink. In some embodiments, an affordance is a toggle button, a checkbox, a radio button, and/or a dropdown list. In some embodiments, an affordance is a manual entry box (e.g., that accepts a user-inputted alphanumeric character and/or an alphanumeric text string).

In some embodiments, the customizable user interface also comprises an affordance for storing the parameters (e.g., as a profile). Storing parameters as test profiles is further described, for instance, in the section entitled “Administrator control,” below, with reference to FIGS. 23A-I.

In some embodiments, one or more mapping statistics in the subset of the plurality of mapping statistics can be modified. In some such embodiments, the method further comprises displaying, on the display, an affordance for amending the subset of the plurality of mapping statistics. For example, in some embodiments, upon user selection of the fourth affordance (e.g., for expanding the subset of the plurality of mapping statistics), the method further comprises displaying, on the display, an affordance for amending the subset of the plurality of mapping statistics. Modifications to the subset of the plurality of mapping statistics, for a respective microorganism, to be displayed on the display can be performed by adjusting one or more parameters, such as test profile parameters 2304 illustrated in FIG. 23D and/or filter parameters 1204 illustrated in FIG. 12.

In some embodiments, one or more sequencing statistics in the subset of the plurality of sequencing statistics can be modified. In some embodiments, the method further comprises displaying, on the display, an affordance for amending the subset of the plurality of sequencing statistics. Modifications to the subset of the plurality of sequencing statistics to be displayed on the display can be performed by adjusting one or more parameters. such as adjustable cutoffs for sequencing statistics 128-1 . . . . , 128-M-3 illustrated in FIG. 23F-H.

In some embodiments, one or more microorganisms in the subset of the set of microorganisms can be modified. In some embodiments, the method further comprises displaying, on the display, an affordance for amending the subset of the set of microorganisms. Modifications to the subset of the set of microorganisms to be displayed on the display can be performed by adjusting one or more parameters. such as selection of relevant subclasses 2306 and/or evidence categories 2308 illustrated in FIGS. 23C and 23I.

In some embodiments, the method further comprises displaying, on the display, an affordance for amending any additional features as described above.

In some embodiments, the method further comprises displaying, on the display, an affordance for amending any of the visual indications as described above (e.g., on the system for review and visualization, the dashboard, and/or the customizable user interface). Amendments to features of the result set analysis, including mapping statistics, sequencing statistics, and/or subsets of microorganisms, are further described herein, e.g., in the sections entitled “Filters” and “Administrator control,” below.

Review Status and Approvals.

As described above, in some embodiments, the customizable user interface 401 comprises an affordance for updating the review status for the nucleic acid sequencing data. In some embodiments, the method further comprises obtaining an approval for the customizable user interface. In some embodiments, the method further comprises obtaining a plurality of approvals for the customizable user interface. For example, in some such embodiments, the analysis of the result set includes accepting one or more approvals (e.g., by a laboratory or medical technician, supervisor and/or director) prior to final approval of the analysis of the results set.

In some embodiments, the customizable user interface comprises an affordance for submitting a review (e.g., for a sample) (e.g., affordance 604). In some embodiments, the customizable user interface comprises an affordance for canceling a review (e.g., for the sample) (e.g., affordance 608). In some embodiments, the customizable user interface comprises an affordance for resetting a review (e.g., to a default state) (e.g., affordance 606). In some embodiments, the affordance for updating the review status is an affordance for initiating a review of the analysis (e.g., review affordance 332 in results dashboard 302).

In some embodiments, each approval stage for a respective sample is indicated by a review status (e.g., review status 440 in customizable user interface 401 and/or status 316 in results dashboard 302). Furthermore, in some embodiments, selection and/or approval at any stage of the approval process (e.g., first, second, third, and/or final approval) can be tagged with a user identity, an access time-stamp, and/or a record of each change made in the respective sample.

In some embodiments, submission of the review updates the review status from a first review status to a second review status. For example, in some embodiments, submission of a review for a sample with a “first review” status updates the review status to “second review”. Similarly, in some embodiments, additional submissions of reviews sequentially change the review status from “second review” to “medical director review,” “final review,” and “approved.” For example, in FIG. 4, a first review status 440-1 is a medical director review (“MD”), where submission of the first review status changes the review status 440 to a second review status 440-2 that is a final review (“Final”).

In some embodiments, final approval of a sample (e.g., a control and/or an analysis sample) removes the sample 304 from the index of biological or non-biological samples 306 (e.g., the list of one or more pending samples). In some such embodiments, when the review status is finally approved, the sample is displayed in a second index of biological or non-biological samples (e.g., a “results history” page) and is no longer visible in the first index of biological or non-biological samples (e.g., the “pending samples” dashboard). In some embodiments, the customizable user interface further comprises a result history comprising at least the first customizable diagnostic template applied to the result set, wherein the review status of the result set is approved.

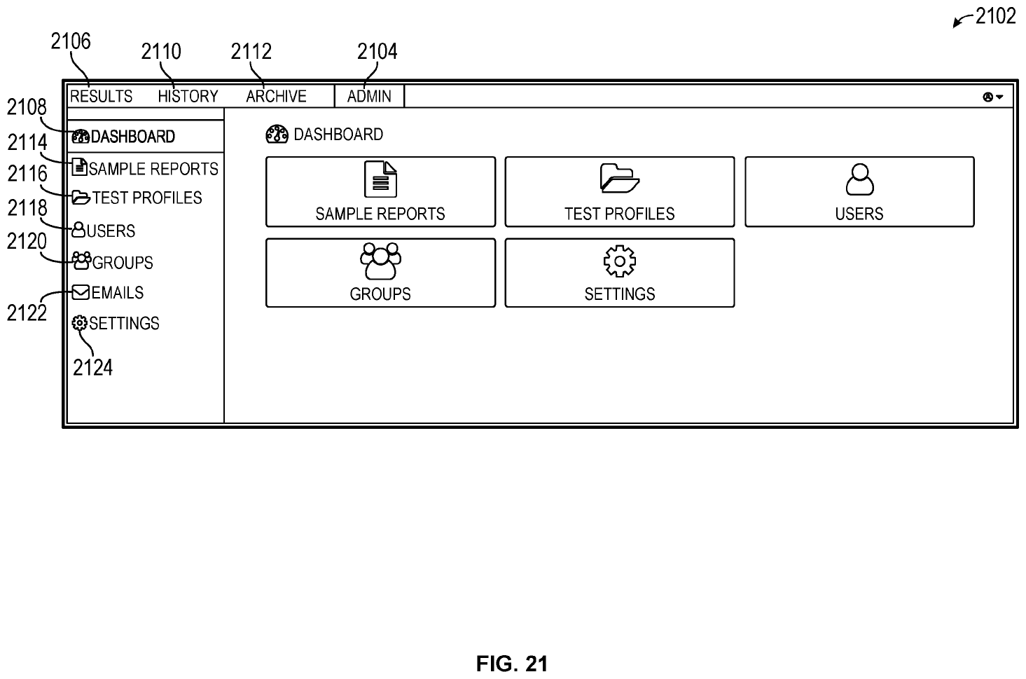

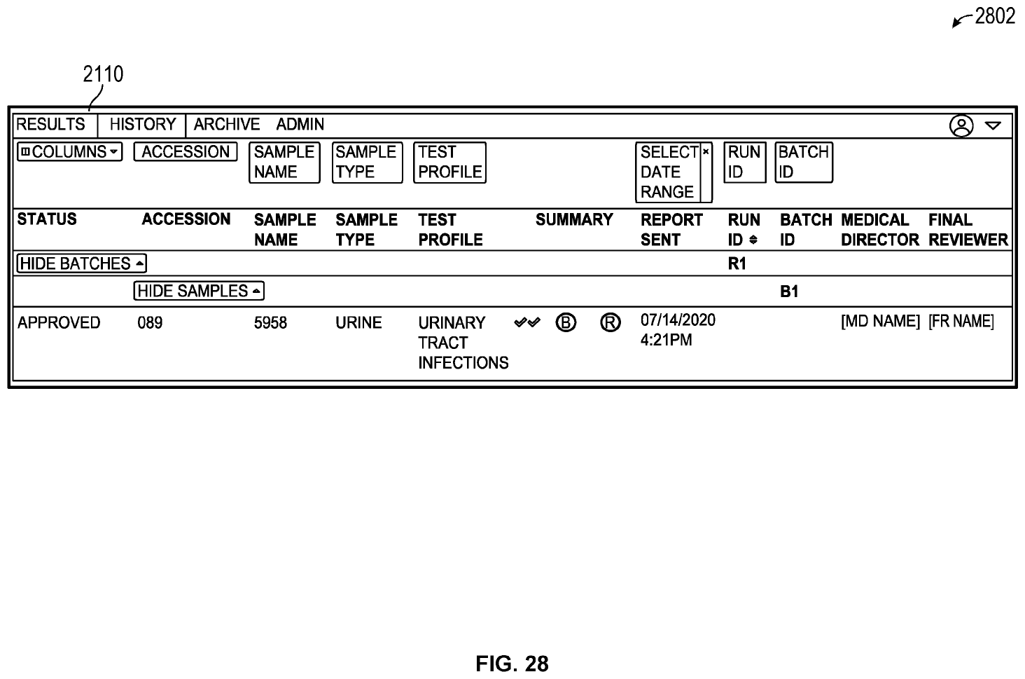

As illustrated in FIG. 21, in some embodiments, samples displayed in the index of biological or non-biological samples 306 (e.g., in a results dashboard 302) can be viewed by selection of a results affordance (e.g., results tab) 2106. Moreover, in some embodiments. samples displayed in a second index of biological or non-biological samples (e.g., a “results history” page 2802) can be viewed by selection of a history affordance (e.g., history tab) 2110. FIG. 28 illustrates a display of an index 2802 of approved samples (e.g., a result history of a review and visualization system) for which review of nucleic acid sequencing data is completed, in accordance with some embodiments of the present disclosure. In some embodiments, the second index of biological or non-biological samples (e.g., the results history page 2802) includes any of the features of the first index of biological or non-biological samples (e.g., the results dashboard 302) disclosed herein, including column headings, run list, batch list, sample list, summary, search boxes, and/or affordances, and/or any substitutions, modifications, additions, deletions, and/or combinations thereof as will be apparent to one skilled in the art. In some embodiments, one or more samples is archived (e.g., stored) in an archive for later viewing and/or modifying. As illustrated in FIG. 21, in some embodiments, samples displayed in the archive can be viewed by selection of an archive affordance 2112.

In some embodiments, any one of the results in the results set can be separately approved or rejected, including the presence or absence of a detected microorganism (e.g., “validated” and/or “passed”), a passing score for a quality control metric (e.g., “passed”), and/or a passing score for a sequencing or mapping statistic compared to a filtering threshold (e.g., “passed”).

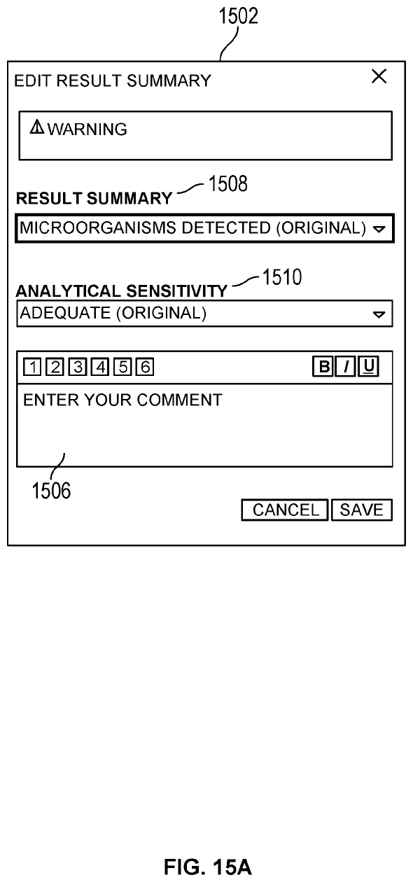

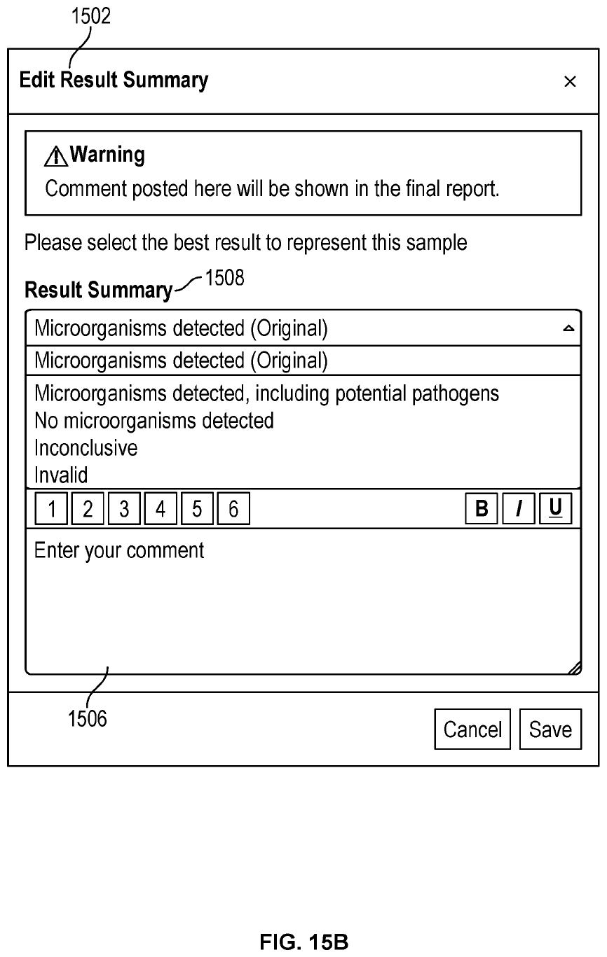

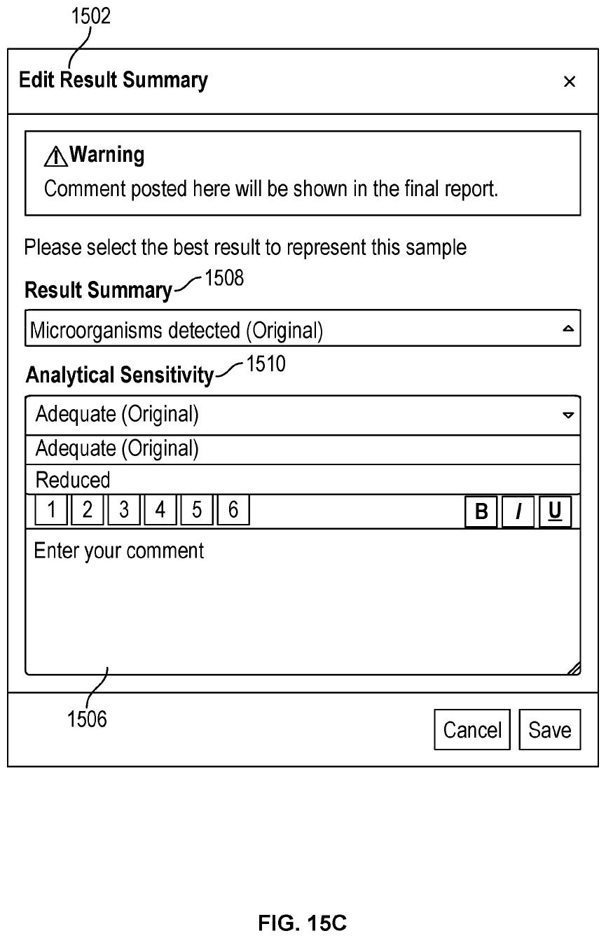

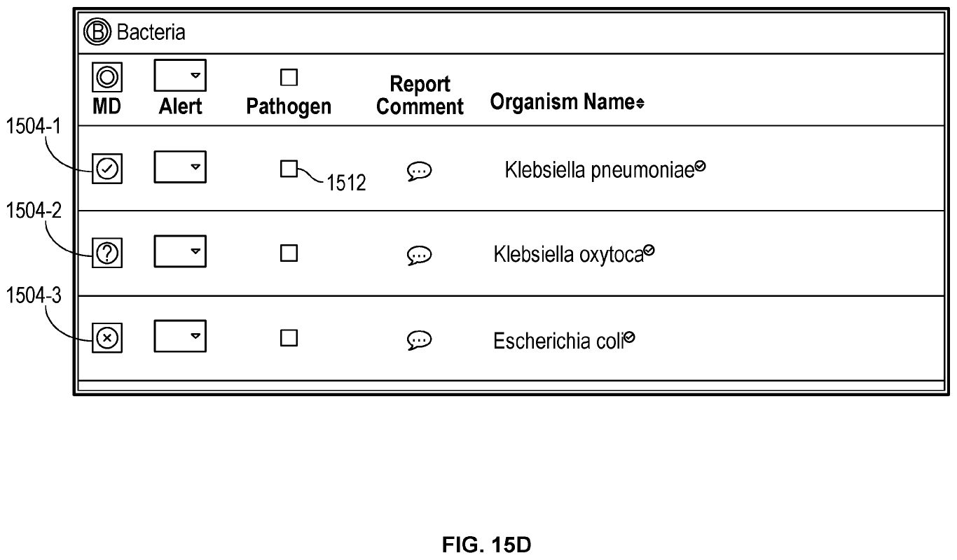

For example, FIG. 15D shows a review status for a review of a microorganism in a customizable user interface for a sample. Visual indicators include representations of review status (e.g., check mark and/or green color to indicate pass 1504-1, X-mark and/or red color to indicate fail 1504-3, and/or question mark and/or yellow color to indicate inconclusive 1504-3).

In some embodiments, upon user selection of the third affordance (e.g., for expanding the summary of the subset of the plurality of sequencing statistics), the method further comprises displaying, on the display, an affordance for validating (e.g., approving or rejecting) the subset of the plurality of sequencing statistics. In some embodiments, upon user selection of the fourth affordance (e.g., for expanding the summary of the subset of the plurality of mapping statistics), the method further comprises displaying, on the display, an affordance for validating (e.g., approving or rejecting) the subset of the plurality of mapping statistics. In some embodiments, a mapping statistic (e.g., a display comprising expanded microorganism information) in the subset of the plurality of mapping statistics can be individually validated. In some embodiments, one or more samples, results, or metrics can be flagged for further review. For example, an affordance 514 for validating and/or displaying a validation status of a subset of the plurality of sequencing statistics 128 and/or a subset of the plurality of mapping statistics 126 for a respective microorganism 402 is illustrated in FIG. 5A.

Comment Function.

In some embodiments, upon user selection of the fourth affordance, the method further comprises displaying, on the display, an affordance for appending a user-inputted text string (e.g., a comment and/or note) to the subset of the plurality of mapping statistics.

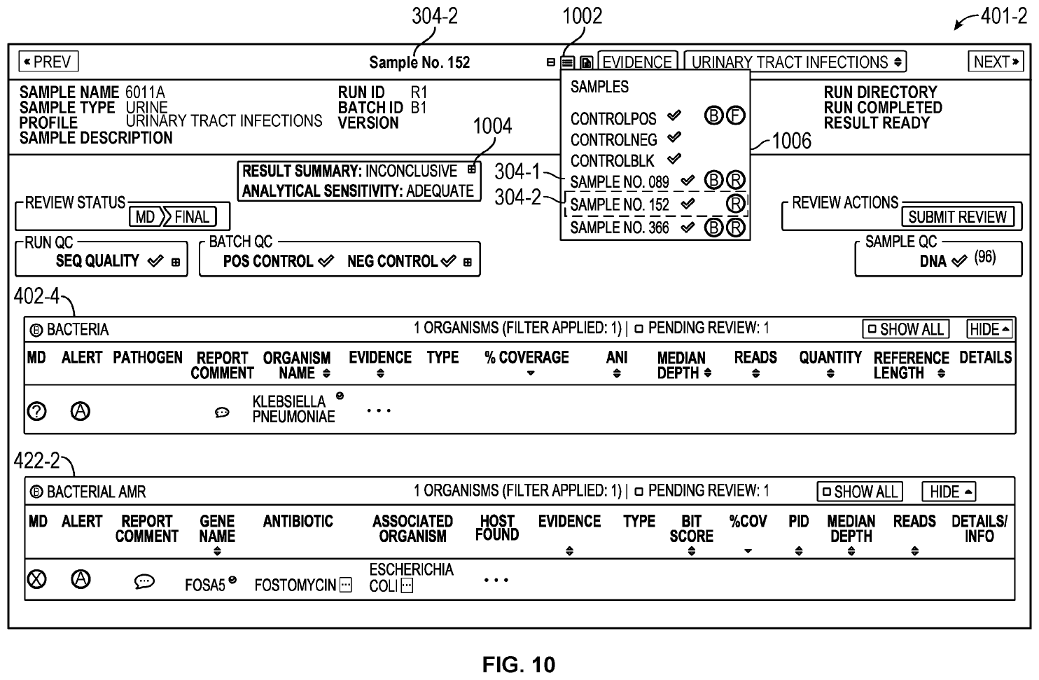

For example, as illustrated in FIGS. 5C, 5D, 6B, 6C, and 15A-C, in some embodiments, the method further comprises displaying, for any of the features displayed in the customizable user interface, an affordance for appending a user-inputted text string. In some embodiments, a user-inputted text string can be appended to, e.g., the customizable user interface, the expanded summary of the subset of the plurality of mapping statistics (e.g., the microorganism viewer), a nucleic acid type for a mapping statistic (e.g., DNA and/or RNA), a nucleic acid type for a sequencing statistic (e.g., DNA and/or RNA), an expanded summary of a subset of a plurality of quality control metrics, and/or an antimicrobial resistance marker (e.g., AMR gene). For example, as described above, in some implementations. an affordance for appending a user-inputted text string includes an affordance for appending a microorganism-associated text string (e.g., microorganism DNA-associated feedback entry affordance 510-1 and/or microorganism RNA-associated feedback entry affordance 510-2). In some implementations, an affordance for appending a user-inputted text string includes an affordance for appending an antimicrobial resistance marker-associated text string (e.g., AMR gene-associated internal notes entry affordance 624 and/or AMR gene RNA-associated feedback entry affordance 626). In some such implementations, the affordance for appending the user-inputted text string is a new display window that is accessed via selection of a comment affordance (e.g., comment affordance 508-1, 508-2, 622-1, 622-2, and/or internal notes affordance 630). In some embodiments, comments can also be added to the result set 122 by selection of a result summary editing affordance (e.g., editing affordance 1004 in FIG. 10). As illustrated in FIGS. 15A-C, selection of editing affordance 1004 displays a new display window, within which an affordance 1506 for appending a user-inputted text string is provided. Embodiments for editing a results summary are further described herein, e.g., in the section entitled “Edit results summary,” below.

In some embodiments, the user-inputted text string is a feedback or an internal note. In some embodiments, affordance for appending a user-inputted text string is accessible to a reviewer e.g., a first, second, third or final reviewer. In some embodiments, a user-inputted text string can be edited. In some embodiments, a user-inputted text string is visible to other users, e.g., a comment provided by a first reviewer is visible to a final reviewer.

Alert Status.

In some embodiments, the customizable user interface includes an alert status indicator (e.g., N: no call: A: alert; C: critical). In some embodiments, the alert status indicator is applied to a microorganism in the subset of the set of microorganisms to flag the respective microorganism for review. FIG. 13 illustrates an affordance 1302 for updating an alert status using a dropdown list.

Quick Access Tools.

In some embodiments, the customizable user interface comprises an affordance for viewing and/or selecting a biological or non-biological sample 304 for analysis of presence of microorganisms. In some such embodiments, the affordance for viewing and/or selecting biological or non-biological samples is accessible from a first customizable user interface of a first biological or non-biological sample. In some embodiments, a selection of a second biological or non-biological sample, using the affordance for viewing and/or selecting biological or non-biological samples, applies the first customizable diagnostic template 138-1 to the selected second biological or non-biological sample and displays a corresponding second customizable user interface for the second biological or non-biological sample. For example, FIG. 10 illustrates an affordance (e.g., dropdown list 1002) for selecting a run, batch, and/or sample for viewing and/or selection, in accordance with some embodiments of the present disclosure. In FIG. 10, the affordance 1002 can be selected from the first customizable user interface 401-2 of a first sample 304-2 (e.g., sample no. 6011A). Selection of the affordance 1002 displays a dropdown list including a plurality of possible samples that can be selected for viewing (e.g., positive control, negative control, blank control, sample 304-1, sample 304-2, sample 304-3, etc.). Accordingly, selection of a second sample 304-1 (e.g., sample no. 5958) from the dropdown list applies the first customizable diagnostic template 138-1 to the selected second sample 304-1 and displays the corresponding second customizable user interface 401-1 for the second sample. In some embodiments, each respective customizable user interface includes a corresponding (i) respective review status for the nucleic acid sequencing data, (ii) a first affordance for updating the review status for the nucleic acid sequencing data, (iii) a respective summary of the subset of the plurality of sequencing statistics 128, (iv) for each respective microorganism in the subset of the set of microorganisms (e.g., the subset of the set of at least 3, at least 5, or at least 10 microorganisms) satisfying a minimum mapping threshold in the respective result set, a corresponding summary of the subset of the plurality of mapping statistics 126 for the respective nucleotide sequences in the plurality of nucleotide sequences mapped to the reference sequence of the respective microorganism, and (v) a second affordance for applying a second customizable diagnostic template 138-2 to the result set. Accordingly, as illustrated in FIGS. 4 and 10, customizable user interfaces 401-1 and 401-2 display respective unique subsets of the set of microorganisms (e.g., 402-1 and 402-4) and respective unique subsets of AMR genes (e.g., 422-1 and 422-2) identified by the analysis.

Customization of User Inter/Ace.

Additional elements that can be customized include specific parameters or metrics to be presented on the display for each sample, batch, and/or run. In some embodiments, an affordance is provided for modifying the display.

For example, in some embodiments, e.g., as illustrated in FIGS. 3A and 3B, modifying the display causes display, for each sample in the index of samples for the user (e.g., the results dashboard 302 and/or sample queue 306), of one or more features 328 for the respective sample selected from the group consisting of a review status, an accession code, a sample name, a sample type, a sample description, a test profile, a sample summary, a run identifier, a batch identifier, a run directory, a run completion time, an analysis platform version, a review platform version, a pipeline version, an analysis version, and an analysis completion time.

In accordance with some embodiments of the present disclosure, FIG. 3B shows an affordance 326 for modifying the display. The affordance can be a dropdown list and/or an overlay display comprising checkboxes. In FIG. 3B, the affordance provides for the selection of one or more features 328 to be displayed on the display. In some embodiments, visibility of various features and/or metrics on the user interface is individually toggled (e.g., by left or right clicking). In some embodiments, expansion of specific subsets of features is performed by clicking on a “+” or “show” button to expand a summary into a detailed view. In some embodiments, minimization of specific subsets of features is performed by clicking on a “−” or “hide” button to minimize a detailed view into a summary.

In some embodiments, an affordance is provided for modifying the subset of the set of microorganisms that is displayed on the customizable user interface. For example, in some embodiments, a user interaction with the affordance causes display, on the customizable user interface, for a microorganism in the set of microorganisms. In some embodiments, a user interaction with the affordance causes display of all of the microorganisms in the set of microorganisms. For example, FIG. 11 shows a customizable user interface 401-1 where an affordance 442 for displaying all of the microorganisms 402 in the set of microorganisms is selected (e.g., “show all” affordance 442). In some embodiments, for each microorganism displayed, the user interface includes a summary of each microorganism in the set of microorganisms. In some embodiments, the summary of each microorganism includes a summary of the plurality of sequencing statistics and, for each microorganism in the set of microorganisms, a summary of the plurality of mapping statistics corresponding to the plurality of nucleotide sequences that map to the reference sequence of the respective microorganism (e.g., statistics 404-418, 422-432).

In some embodiments, a user interaction with the affordance (e.g., “show all” affordance 442) displays all of the microorganisms included in the result set (e.g., all microorganisms to which the plurality of nucleotide sequences were mapped). For example, FIG. 11 shows the plurality of microorganisms in the result set, where the plurality of microorganisms in the result set is not filtered by applying the first customizable diagnostic template 138-1. In some embodiments, displaying all of the microorganisms included in the result set displays one or more microorganisms that fail to satisfy the cutoff threshold for detection applied by the first customizable diagnostic template, in addition to the microorganisms that satisfy the cutoff threshold for detection. For example, FIGS. 12 and 13 illustrate a customizable user interface 401-2 where an affordance 442 for displaying all of the microorganisms 402 in the set of microorganisms is selected (e.g., “show all”). In this instance, the percent coverage mapping statistic (e.g., 11%) for the respective microorganism Kiebsiella aerogenes fails to satisfy the cutoff threshold for detection (e.g., 72%), displayed as an overlay display 1304. In some such embodiments, microorganisms that fail to satisfy the cutoff thresholds for one or more statistics are displayed such that the respective statistics are visually distinct from those that satisfy the cutoff thresholds (e.g., a different shade, color, texture, etc.) and are removed from display when the affordance 442 (“Show All”) is deselected. Thus, selecting the affordance to display all of the microorganisms (e.g., affordance 442) displays the set of microorganisms including those that do and do not satisfy one or more cutoff thresholds for detection.

Search Function.

In some embodiments, an affordance is provided for selecting, from the one or more biological or non-biological samples in the index of biological or non-biological samples, a biological or non-biological sample based on an input (e.g., a value) for a respective feature in one or more features of the biological or non-biological sample.

For example, in some embodiments, the user interface includes, for each feature in the one or more features, an affordance for applying a filter to the index of biological or non-biological samples, based on an input for the respective feature.

In an example, FIG. 3A illustrates a search function using manual entry boxes 330 (e.g., 330-1, 330-2, 330-3, etc.), which can be used to filter the plurality of samples by searching for a value or a text-string in any desired feature of the sample, such as a sample accession number, sample type, run identifier, batch identifier, and/or date range. The feature of the sample can be any one of the plurality of sequencing statistics, the plurality of mapping statistics, the plurality of additional metrics, and/or the plurality of quality control metrics. The search function can be performed to search for specific runs, batches, and/or samples displayed on the dashboard.

Addition of Microorganisms.

In some embodiments, an affordance is provided for adding a microorganism to the set of microorganisms. In some embodiments, an affordance is provided for adding a microorganism to the subset of the set of microorganisms.

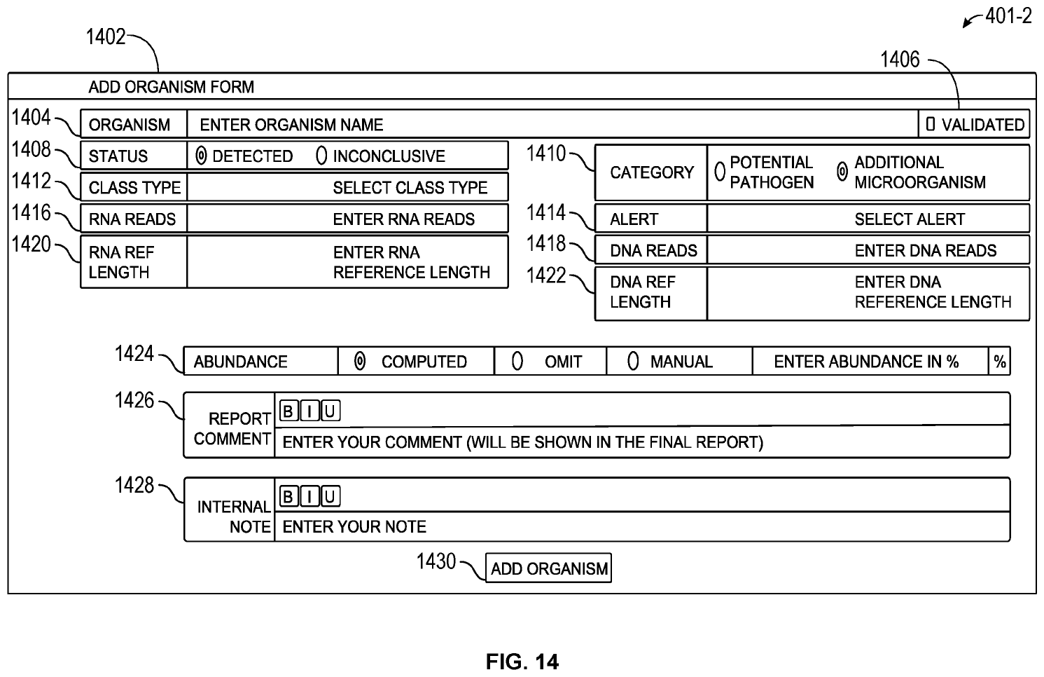

FIG. 14 illustrates an example of an affordance (“Add Organism Form”) 1402 for adding a microorganism to the subset of the set of microorganisms, which is displayed in the customizable user interface 401 (e.g., 401-2). In some embodiments, the organism name is added by a user selection of an entry in a list of entries (e.g., from a dropdown list and/or a checkbox list). In some embodiments, the organism name is added by manual entry of a text-string 1404.

In some embodiments, the affordance 1402 includes an affordance 1408 for assigning a detection status to the microorganism (e.g., detected and/or inconclusive). In some embodiments, the affordance 1402 includes an affordance for assigning a category 1410 to the microorganism (e.g., potential pathogen and/or additional microorganism). In some embodiments, the affordance 1402 includes an affordance 1406 for assigning a validation status to the microorganism (e.g., validated and/or not validated). In some embodiments, the affordance 1402 includes an affordance 1414 for assigning an alert to the microorganism (e.g., no alert, alert, and/or critical). In some embodiments, the affordance 1402 includes an affordance 1424 for assigning an abundance status to the microorganism (e.g., computed, omitted, and/or manual). In some embodiments, the affordance 1402 includes an affordance for assigning an abundance value to the microorganism (e.g., a percentage). In some embodiments, the affordance 1402 includes an affordance 1412 for assigning a class type to the microorganism. In some embodiments, the affordance 1402 includes an affordance 1416 for assigning a number of RNA reads to the microorganism. In some embodiments, the affordance 1402 includes an affordance 1420 for assigning an RNA reference length to the microorganism. In some embodiments, the affordance 1402 includes an affordance 1418 for assigning a number of DNA reads to the microorganism. In some embodiments, the affordance 1402 includes an affordance 1422 for assigning a DNA reference length to the microorganism. In some embodiments, the affordance 1402 includes an affordance 1426 for assigning a report comment to the microorganism. In some embodiments, the affordance 1402 includes an affordance 1428 for assigning an internal note to the microorganism.

In some embodiments, a feature and/or a value for the respective feature is added to the microorganism by a user selection of an entry in a list of entries (e.g., from a dropdown list and/or a checkbox list). In some embodiments, the feature and/or a value for the respective feature is added to the microorganism by manual entry of a text-string. In some embodiments, the affordance 1402 (e.g., “Add Organism Form”) for adding a microorganism to the subset of the set of microorganisms further includes an affordance 1430 for finalizing and submitting the added organism to the subset of the set of microorganisms (e.g., “Add Organism”).

Edit Results Summary.

In some embodiments, the customizable user interface includes a result summary including a status of the analysis of the result set based on the plurality of mapping statistics for each respective microorganism in the set of microorganisms, where the status is selected from the group consisting of: invalid (e.g., no organisms detected and/or failed total IC norm reads), inconclusive, microorganisms detected, microorganisms detected including potential pathogens, and no microorganisms detected. In some embodiments, the customizable user interface includes a status of an analytical sensitivity based on the mapping of the plurality of nucleotide sequences against the reference sequences of the set of microorganisms. In some embodiments, the analytical sensitivity status is adequate or reduced. FIG. 4 illustrates a customizable user interface 401-1 including a result summary 452 that indicates a status of the analysis of the result set (“inconclusive”) and a status of an analytical sensitivity (“adequate”). In some embodiments, the customizable user interface further includes an affordance for displaying and/or modifying a result summary. For example, affordance 1004 in FIG. 10 provides an example of an affordance for displaying and modifying a result summary. In some embodiments, selection of the affordance 1004 generates a new display window in which the result summary can be modified and/or in which an annotation or comment can be added to the result summary. For example, FIGS. 15A, 15B, and 15C illustrate an embodiment where selection of an affordance 1004 generates a display window 1502 for displaying and modifying a result summary. The display window 1502 includes one or more affordances for modifying the result summary 1508 and/or modifying the analytical sensitivity 1510. Selection of affordance 1508 displays a dropdown list of result summary options (e.g., invalid (e.g., no organisms detected and/or failed total IC norm reads), inconclusive, microorganisms detected, microorganisms detected including potential pathogens, and/or no microorganisms detected). Selection of affordance 1510 displays a dropdown list of analytical sensitivity options (e.g., adequate and/or reduced). In some embodiments, the result summary further comprises an affordance 1506 for adding a comment, feedback, and/or internal note to the result summary.

Pathogen Status.

In some embodiments, an affordance is provided for indicating a pathogen status (e.g., pathogen or not pathogen) for a microorganism in the subset of the set of microorganisms. For example, FIG. 15D illustrates an affordance for indicating a pathogen status for a microorganism in accordance with some embodiments of the present disclosure, where the customizable user interface displays an affordance (e.g., checkbox 1512) that is selected or unselected to indicate the pathogen status.

Export Results.

In some embodiments, the customizable user interface 401 further includes an affordance for exporting a summary of the analysis of the results set 122. In some embodiments, the customizable user interface 401 further includes an affordance for previewing an exported summary of the analysis of the results set 122. In some embodiments, the customizable user interface 401 further includes an affordance for generating a report of the analysis of the results set 122. In some embodiments, the customizable user interface 401 further includes an affordance for previewing a report of the analysis of the results set 122. In some embodiments, the exported results include results for a respective biological or non-biological sample 304. In some embodiments, the exported results include results fora respective organism (e.g., microorganism 402).

An exported summary or a report can be customized by selecting the features to be included. In some embodiments, the customizable user interface includes an affordance for selecting, for the previewing of the exported summary, the subset of the plurality of sequencing statistics and the subset of the plurality of mapping statistics from the results set. In some embodiments, the customizable user interface further includes an affordance for selecting, for the report, the subset of the plurality of sequencing statistics and the subset of the plurality of mapping statistics from the results set to be included in the report.

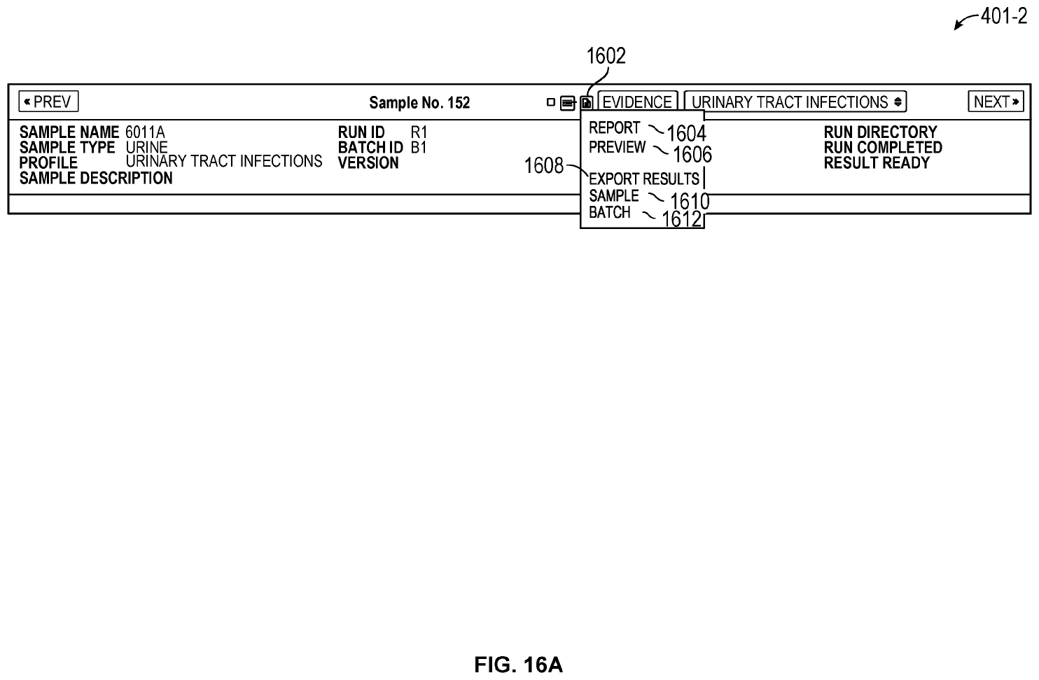

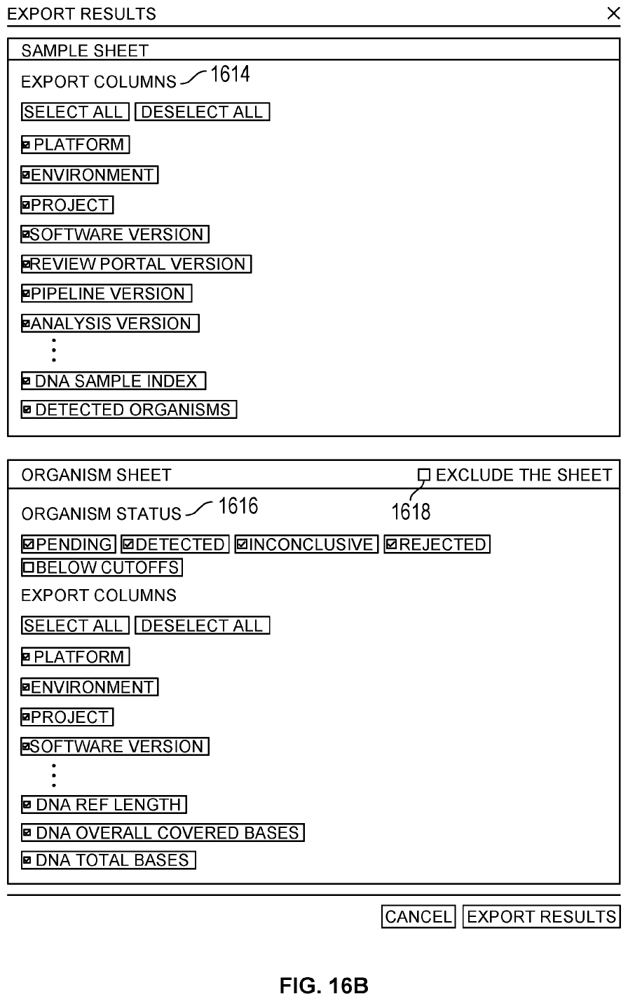

For example, FIG. 16A illustrates an affordance 1602 for selecting from a plurality of reporting actions, including an affordance 1608 for exporting a summary of the results, an affordance 1606 for previewing the exported summary, and/or an affordance 1604 for generating a report and/or previewing the report. FIG. 16A further illustrates affordances for selecting the features (e.g., sample-level features 1610 and/or batch-level features 1612) to be included in the exported results, the report, and/or the preview thereof. In some embodiments, the affordances for selecting the features to be included in the exported results and/or the report allows a user to select features for a biological or non-biological sample 1610. In some embodiments, the affordance for selecting the features to be included in the exported results and/or the report allows a user to select features for a batch of biological or non-biological samples 1612.

In some embodiments, as illustrated in FIG. 16B, upon receiving a user interaction with an affordance for a reporting action (e.g., exporting results affordance 1608), the method comprises displaying a plurality of features that can be selected or deselected. In some embodiments, a plurality of features can be selected or deselected for a respective sample 1614 (e.g., for a sample report). In some embodiments, a plurality of features can be selected or deselected for a respective organism 1616 (e.g., microorganism). In some embodiments, an affordance 1618 is provided for excluding one or more data sets from the exported results and/or the report.

In some embodiments, the plurality of features that can be selected or deselected for inclusion in the exported results and/or the report (e.g., using feature selection displays 1614 and/or 1616) include any one or more of the sequencing statistics, mapping statistics, additional metrics, quality control metrics, set of microorganisms, and/or other metadata associated with a respective sample, batch, or run as disclosed herein. In some embodiments, the plurality of features that can be selected or deselected for inclusion in the exported results and/or the report (e.g., using feature selection displays 1614 and/or 1616) include: a platform, environment, project, software version (e.g., Explify version), review portal version, analysis pipeline version, analysis version, run ID, run directory, run start time, run completion time, batch ID, results directory, total run yield, percent bases that pass a Q30 threshold, cluster density, percent clusters passing a filter, PhiX error rate, percent of sequencing tiles that pass a selection criterion, intensity A, intensity C, chemistry, instrument ID, accession number, sample ID, sample name, sample type, results ready time, MD review start time, MD review completion time, report transmission time, positive control ID, positive control lot, negative control ID, negative control lot, RNA IC ID, RNA IC lot, RNA MS2 norm reads, RNA MS2 raw reads, RNA Qbeta norm reads, RNA Qbeta raw reads, DNA IC ID, DNA IC lot, DNA T7 norm reads, DNA T7 raw reads, DNA PR772 norm reads, DNA PR772 raw reads, RNA library type, RNA library name, RNA seq sample, RNA total raw reads, RNA post-adaptor reads, RNA post-quality reads, RNA unique reads. RNA percent unique reads, RNA entropy, RNA G content, RNA library Q score, RNA library size, RNA library concentration, RNA sample index, DNA library type, DNA library name, DNA seq sample, DNA total raw reads, DNA post-adaptor reads, DNA post-quality reads, DNA unique reads, DNA percent unique reads. DNA entropy, DNA G content, DNA library Q score, DNA library size, DNA library concentration, DNA sample index, and/or detected organisms.

In some embodiments, the plurality of features that can be selected or deselected for inclusion in the exported results and/or the report (e.g., using feature selection displays 1614 and/or 1616) further include: an organism name, class type, subclasses, reporting ID, review information, positive control organism name, potential pathogen information, medically relevant information, validation information. passed cutoff information, nucleic acid information, antibiotic information, associated organisms, host detection status, RNA percent coverage, RNA sensitivity cutoff, RNA specificity cutoff, RNA bit score, RNA bit score cutoff, RNA average nucleotide identity. RNA median depth, RNA reads, RNA quantity, RNA reference length, RNA overall covered bases, RNA total bases, DNA percent coverage, DNA sensitivity cutoff, DNA specificity cutoff, DNA bit score, DNA bit score cutoff, DNA average nucleotide identity, DNA median depth, DNA reads, DNA quantity. DNA reference length, DNA overall covered bases, and/or DNA total bases.

In some embodiments, any of the features disclosed in the foregoing paragraphs can be modified or customized via user interaction.

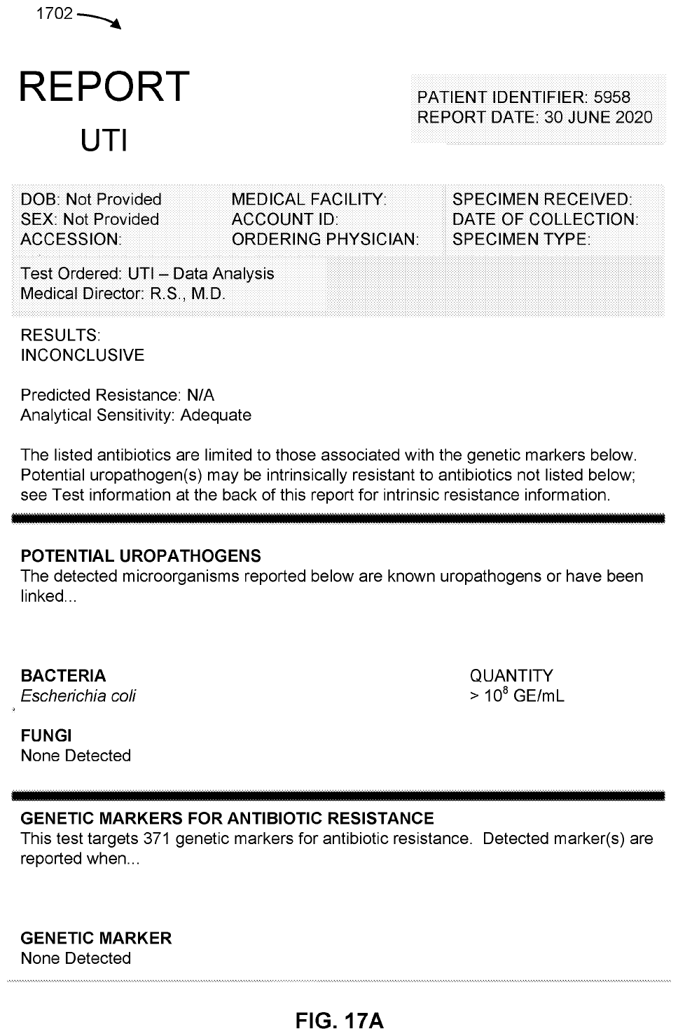

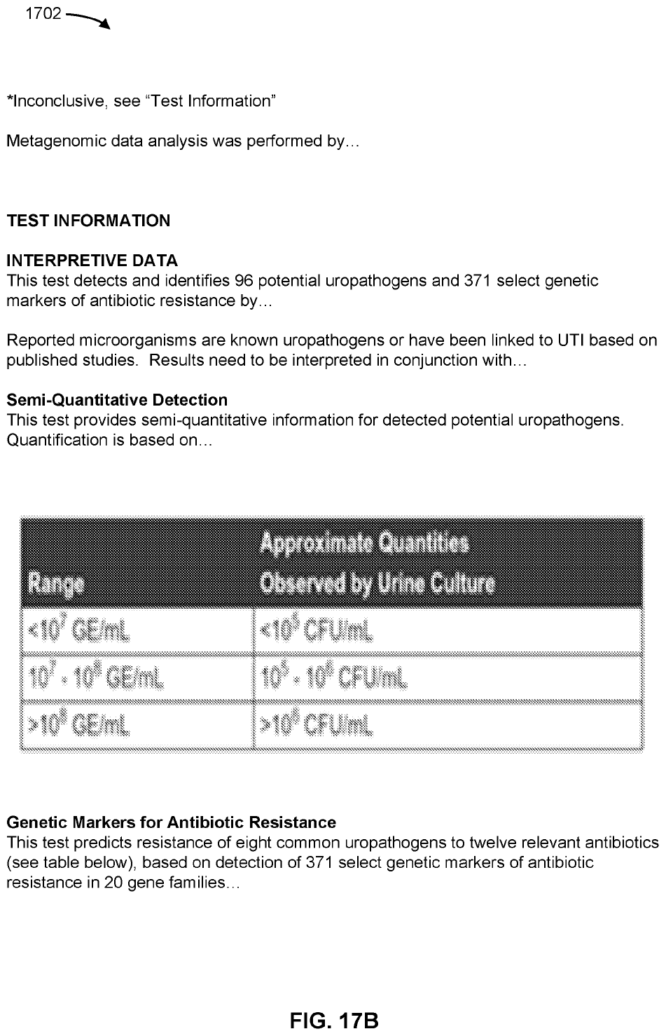

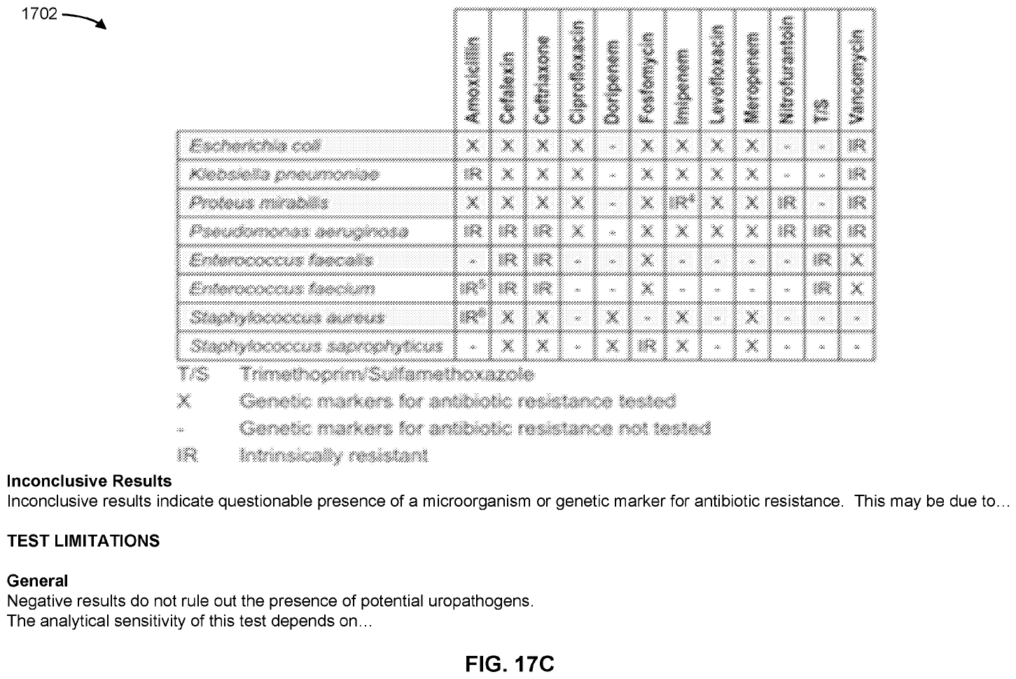

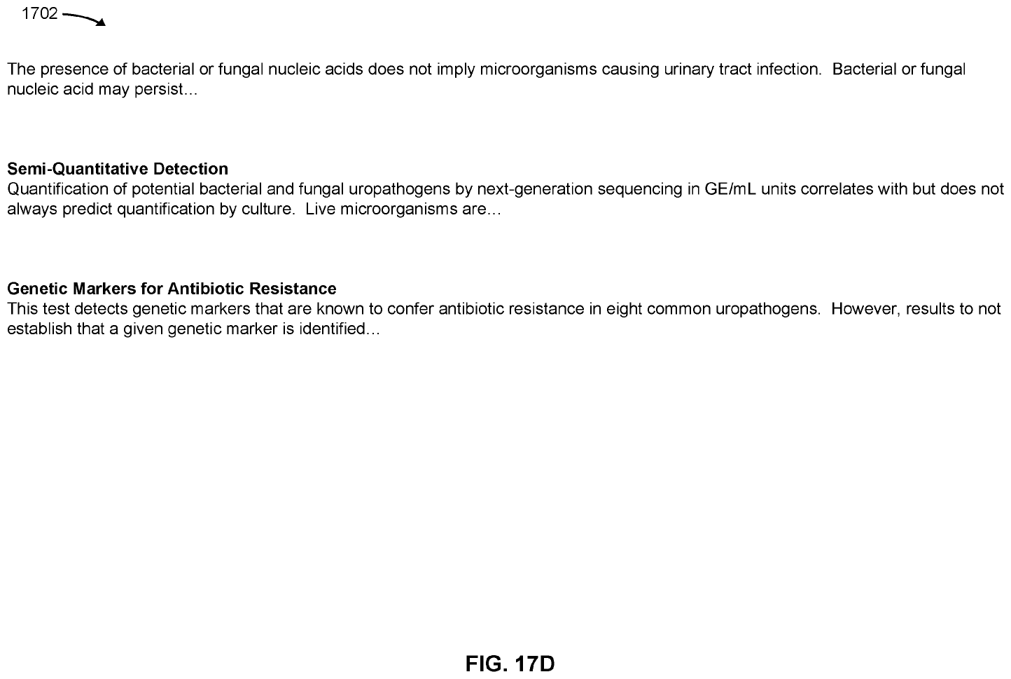

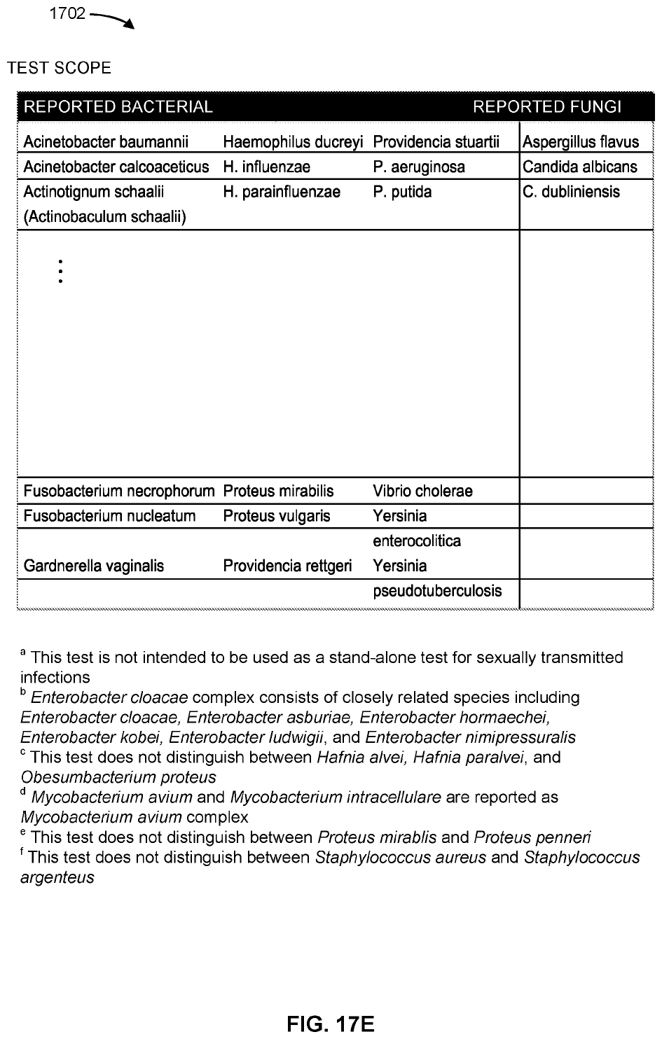

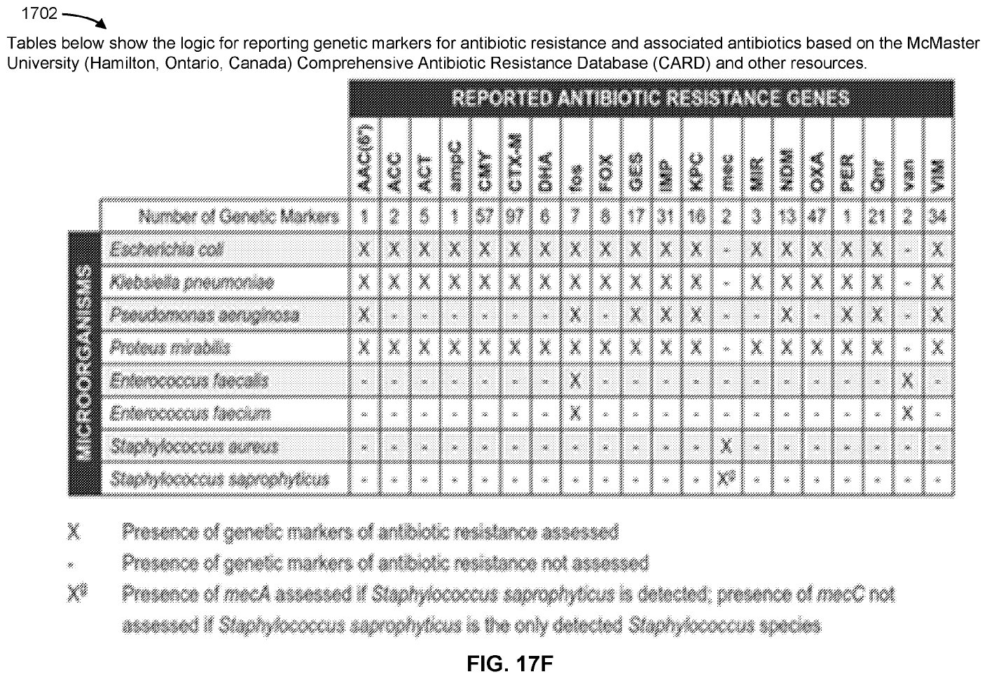

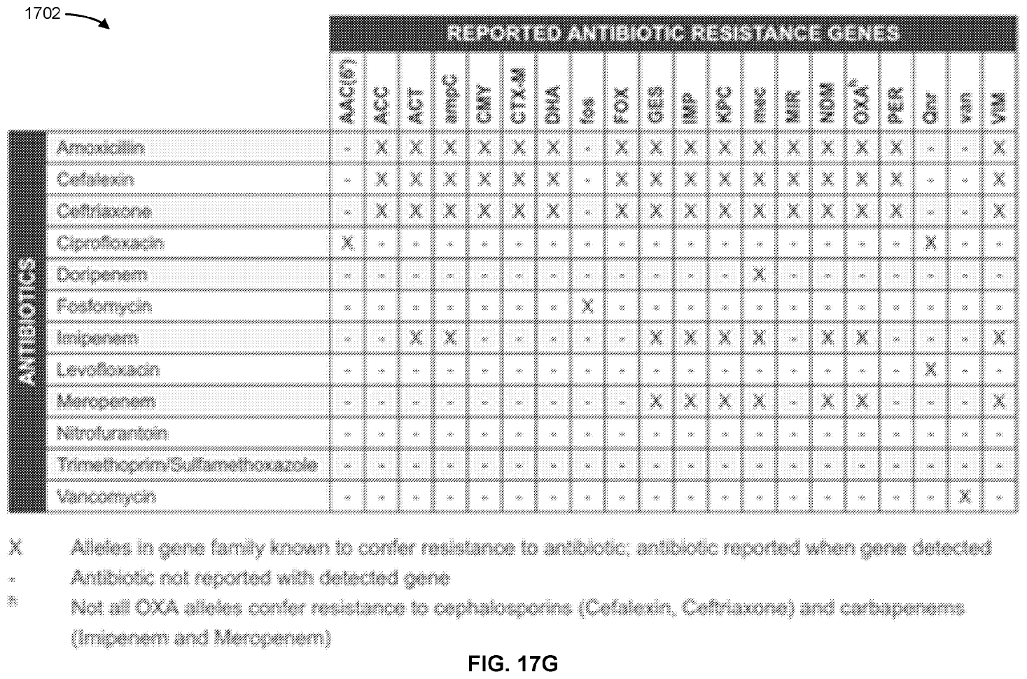

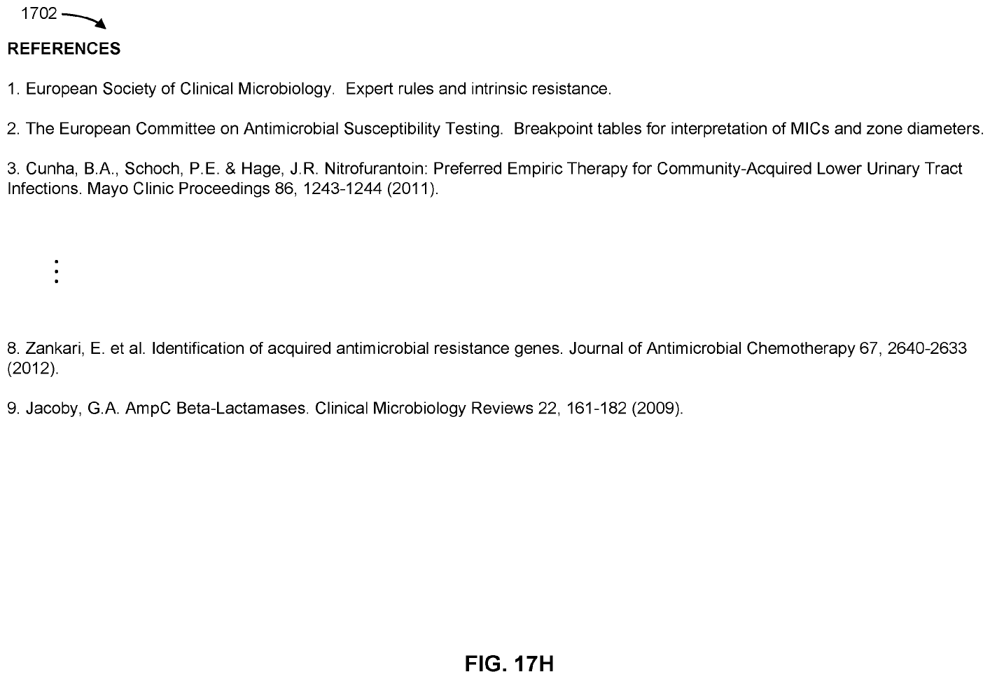

In some embodiments, other features are customizable and/or user interactive, as will be apparent to one skilled in the art. In some embodiments, the customization and/or user interaction is performed using any of the user inputs and/or affordances disclosed herein, and/or any substitutions, modifications, additions, deletions, and/or combinations thereof. In some embodiments, the method includes, upon selection of an affordance for a reporting action (e.g., report generation 1604 and/or exporting results 1608), generating a report. Report generation is further described herein, e.g., in the section entitled “Report generation,” below, with reference to FIGS. 17A-H.

Filters.

As described above, in some embodiments, the second customizable diagnostic template includes a plurality of filters for filtering the result set for the biological or non-biological sample, based on one or more features. For example, the second customizable template can be applied to the result set to further limit the result set to display information related to specific microorganisms, specific pathogens, specific disease conditions, and/or any other feature of interest. In some embodiments, the second customizable template can be applied to the result set to further limit the result set to display information that passes one or more cutoff thresholds. As illustrated in FIGS. 11 and 12, an example second affordance 1104 (e.g., “Filter”) displays one or more filters that are applied to the (i) the subset of the plurality of sequencing statistics 128, (ii) the subset of the set of microorganisms, and/or (iii) for each respective microorganism in the subset of the set of microorganisms, the corresponding subset of the plurality of mapping statistics 126. For example, in some implementations, selection of the second affordance 1104 applies the second customizable diagnostic template 138-2 to the result set, where the second customizable diagnostic template includes one or more parameters for filtering the sequencing statistics, microorganisms, and/or mapping statistics for display. In some embodiments, as illustrated in FIG. 12, selection of the second affordance 1104 (e.g., “Filter”) expands the customizable user interface 401-2 to display a plurality of filter criteria (e.g., filter parameters 1204). In some embodiments, as illustrated in FIG. 13, selection of the second affordance 1104 (e.g., “Filter”) minimizes the customizable user interface 401-2 to hide the plurality of filter criteria (e.g., filter parameters 1204).

In some embodiments, upon user selection of the second affordance, the method further comprises applying the second customizable diagnostic template to the result set by applying a filter to the subset of the plurality of sequencing statistics, the subset of the set of microorganisms, and the subset of the plurality of mapping statistics.

In some embodiments, the second customizable diagnostic template includes a disease condition filter, a microorganism in the set of microorganisms is annotated with the disease condition based on a threshold number of co-occurrences (e.g., evidence 1206) of the microorganism and the disease condition in a database (e.g., a disease annotation in a database), and the applying the filter selectively retains one or more microorganisms annotated with the disease condition.

In some embodiments, the threshold number of co-occurrences of the microorganism is at least 1, at least 2, at least 3, at least 4, at least 5, at least 6, at least 7, at least 8, at least 9, at least 10, at least 50, at least 100, at least 500, at least 1000, at least 2000, or at least 5000.

In some embodiments, the disease condition is an infectious disease. In some embodiments, the disease condition is a medically relevant condition (e.g., “Medically Relevant” affordance 1208). In some embodiments, the disease condition is a disease caused by a pathogen. In some embodiments, the disease condition is a disease caused by a microorganism. In some embodiments, the disease condition is a brain infection, urinary tract disease, respiratory disease, CNS, and/or cancer.

In some embodiments, the disease condition is influenza, common cold, measles, rubella, chickenpox, norovirus, polio, infectious mononucleosis (mono), herpes simplex virus (HSV), human papillomavirus (HPV). human immunodeficiency virus (HIV). viral hepatitis (e.g., hepatitis A, B, C, D, and/or E), viral meningitis, West Nile Virus, rabies, Ebola, strep throat, bacterial urinary tract infections (UTIs) (e.g., coliform bacteria), bacterial food poisoning (e.g., E. coli, Salmonella, and/or Shigella), bacterial cellulitis (e.g., Staphylococcus aureus (MRSA)), bacterial vaginosis, gonorrhea. chlamydia, syphilis, Clostridium difficile (C. diff), tuberculosis, whooping cough, pneumococcal pneumonia. bacterial meningitis. Lyme disease, cholera, botulism, tetanus, anthrax, vaginal yeast infection, ringworm, athlete's foot, thrush, aspergillosis, histoplasmosis, Cryptococcus infection, fungal meningitis, malaria, toxoplasmosis, trichomoniasis, giardiasis, tapeworm infection, roundworm infection, pubic and head lice, scabies, leishmaniasis, and/or river blindness.

In some embodiments, the disease condition is a viral respiratory disease. In some embodiments, the disease condition is a coronavirus infection. In some embodiments, the disease condition is a SARS-CoV-2 infection.

In some embodiments, the second customizable diagnostic template includes a target microorganism filter, and the applying the filter selectively retains one or more microorganisms that share at least a threshold sequence identity to the target microorganism. For example, in some embodiments, the threshold is customized to selectively retain, from the result set, a plurality of pathogens including a first pathogen and a second pathogen that is genetically similar to the first pathogen (e.g., based on a sequence identity, a class, a parentage, and/or a phylogenetic lineage). In some embodiments, the threshold sequence identity is between 0 and 10%. between 10 and 20%, between 20 and 30%, between 30 and 40%, between 40 and 50%, between 50 and 60%, between 60 and 70%, between 70 and 80%, between 80 and 90%, or between 90 and 100%. In some embodiments, the threshold sequence identity is at least 70%, at least 80%, at least 90%, at least 95%, at least 96%, at least 97%, at least 98%, or at least 99%. In some embodiments, the applying the filter comprises manually entering a numeric value for a threshold sequence identity. In some embodiments, the applying the filter comprises manually entering a microorganism class to be selectively retained. In some embodiments, the applying the filter comprises manually entering a microorganism name to be selectively retained (e.g., organism name search 1210). In some embodiments, the applying the filter comprises manually entering a microorganism parent name to be selectively retained. In some embodiments, the applying the filter comprises manually entering a phylogenetic lineage to be selectively retained (e.g., phylogenetic lineage search 1212).

In some embodiments, the second customizable diagnostic template includes an antimicrobial resistance filter, the applying the filter selectively retains one or more microorganisms, and the mapping of the respective nucleotide sequences in the plurality of nucleotide sequences to the reference sequence for the respective microorganism indicates the presence of an antimicrobial resistance marker (e.g., where an AMR gene is based on an annotation and/or a platform-curated genome library).

In some embodiments, the second customizable diagnostic template includes a mapping statistics filter (e.g., RNA filters 1214 and/or DNA filters 1216), and the applying the filter selectively retains one or more microorganisms having at least a threshold value for a mapping statistic in the plurality of mapping statistics (e.g., coverage, depth, sample type, tissue of origin, nucleic acid type, number of reads, reference length, ANI, bit score, and/or PID).

In some embodiments, the second customizable diagnostic template includes an annotation filter, where the result set is filtered by manually entering a text string (e.g., a search term) to be selectively retained.

In some embodiments, the second customizable diagnostic template includes a run metrics filter, where the result set is filtered based on one or more run metrics.

In some embodiments, the second customizable diagnostic template includes a mapping statistics filter, where the result set is filtered based on one or more mapping statistics in the plurality of mapping statistics.

In some embodiments, the second customizable diagnostic template includes a sequencing statistics filter, where the result set is filtered based on one or more sequencing statistics in the plurality of sequencing statistics.

In some embodiments, the second customizable diagnostic template includes an additional metrics filter, where the result set is filtered based on one or more additional metrics in the plurality of additional metrics.

In some embodiments, the second customizable diagnostic template includes a quality control metrics filter, where the result set is filtered based on one or more quality control metrics in the plurality of quality control metrics.

In some embodiments, the filter is based on any of the features disclosed herein, that are displayed on a display, a dashboard (e.g., results dashboard 302), a sample viewer (e.g., customizable user interface 401), an organism viewer (e.g., expanded microorganism display 502), a sequencing statistics viewer (e.g., expanded sequencing statistics display), a mapping statistics viewer (e.g., expanded mapping statistics display), a quality control metrics viewer (e.g., expanded quality control display), and/or an AMR gene viewer (e.g., expanded AMR gene display 602).

In some embodiments, the filter is based on one or more features, including: a platform, environment, project, software version (e.g., Explify version), review portal version, analysis pipeline version, analysis version, run ID, run directory, run start time, run completion time, batch ID, results directory, total run yield, percent bases that pass a Q30 threshold, cluster density, percent clusters passing a filter, PhiX error rate, percent of sequencing tiles that pass a selection criterion, intensity A, intensity C, chemistry, instrument ID, accession number, sample ID, sample name, sample type, results ready time, MD review start time, MD review completion time, report transmission time, positive control ID, positive control lot, negative control ID, negative control lot, RNA IC ID, RNA IC lot, RNA MS2 norm reads, RNA MS2 raw reads, RNA Qbeta norm reads, RNA Qbeta raw reads, DNA IC ID, DNA IC lot, DNA T7 norm reads, DNA T7 raw reads, DNA PR772 norm reads, DNA PR772 raw reads, RNA library type, RNA library name, RNA seq sample, RNA total raw reads, RNA post-adaptor reads, RNA post-quality reads. RNA unique reads, RNA percent unique reads, RNA entropy, RNA G content, RNA library Q score, RNA library size, RNA library concentration, RNA sample index, DNA library type, DNA library name, DNA seq sample, DNA total raw reads, DNA post-adaptor reads, DNA post-quality reads, DNA unique reads, DNA percent unique reads, DNA entropy, DNA G content, DNA library Q score, DNA library size, DNA library concentration, DNA sample index, and/or detected organism.

In some embodiments, the filter is based on one or more features, including: an organism name, class type, subclasses, reporting ID, review information, positive control organism name, potential pathogen information, medically relevant information, validation information, passed cutoff information, nucleic acid information, antibiotic information, associated organisms, host detection status, RNA percent coverage, RNA sensitivity cutoff, RNA specificity cutoff. RNA bit score, RNA bit score cutoff, RNA average nucleotide identity. RNA median depth, RNA reads, RNA quantity. RNA reference length, RNA overall covered bases, RNA total bases, DNA percent coverage, DNA sensitivity cutoff, DNA specificity cutoff, DNA bit score, DNA bit score cutoff. DNA average nucleotide identity, DNA median depth, DNA reads, DNA quantity. DNA reference length, DNA overall covered bases, and/or DNA total bases.

In some embodiments, a parameter (e.g., a parameter in the plurality of filtering parameters 1204) for filtering the plurality of sequencing statistics, the set of microorganisms, and the plurality of mapping statistics is selected using an affordance (e.g., a user-interactive affordance). In some embodiments, the affordance is a slider bar, a scroll bar, a dropdown list, a checkbox, a manual entry box (e.g., number, percentage, and/or an alphanumeric text string), a radio button, and/or a toggle button.

In some embodiments, the second customizable diagnostic template includes one or more stored parameters (e.g., filtering parameters 1204) specifying the filter, the subset of the set of microorganisms, and the subset of the plurality of mapping statistics.

In some embodiments, the one or more parameters (e.g., filtering parameters 1204) are stored as a template (e.g., a profile), such as a customizable diagnostic template. In some embodiments, a template is applied to a plurality of result sets (e.g., for a corresponding plurality of samples). For example, a template can be applied to one or more control samples and one or more analysis samples in a batch, thus creating consistency in the analysis between the control samples and the analysis samples. Similarly, a template can be applied to a plurality of analysis samples obtained from a single patient, or from a plurality of patients enrolled in a clinical study.

In some embodiments, the customizable user interface comprises at least 1, at least 2, at least 3, at least 4, at least 5, at least 6, at least 7, at least 8, at least 9, at least 10, at least 20, at least 50, at least 100, at least 200, or more than 200 customizable diagnostic templates. In some embodiments, a respective customizable diagnostic template is stored as a test profile (e.g., as is further described in the section entitled “Administrator control.” below, with reference to FIGS. 23A-1).

In some embodiments, a plurality of analyses is performed for a respective biological or non-biological sample, where for each different analysis in the plurality of analyses. a corresponding different template in a plurality of templates is applied to the biological or non-biological sample (e.g., multiple profiles can be applied to a single result set).

Administrator Control.

In some instances, further customization is also possible through an administrator access account (e.g., administrator account 2502-1), by controlling and managing filters, profiles (e.g., test profiles 2116), user accounts (e.g., users 2118), groups (e.g., groups 2120), and/or permissions for specific users (e.g., granting review and/or approval access). For example, in some implementations, a production workflow can be established by restricting access to analysis samples until one or more control samples are finally approved. In some embodiments, specific filters or profiles can be established for specific scenarios, such as in instances where it is desirable to develop, optimize and validate a user-modified, custom set of parameters and detection thresholds that is subsequently applied, consistently, to all future samples in the workflow.

Dashboard In some embodiments, the method further comprises displaying, on the display, a user interface 2102 for an administrator access account 2502-1. For example, in some embodiments, the receiving a request to display an analysis of a result set 122 obtained from a sequencing reaction of nucleic acids from the biological or non-biological sample 304 comprises receiving log-in credentials for an administrator account 2502-1 and displaying a user interface 2102 for the administrator account. In some embodiments, the receiving a request to display an analysis of a result set 122 obtained from a sequencing reaction of nucleic acids from the biological or non-biological sample 304 comprises receiving log-in credentials for an administrator account 2502-1, displaying an index of biological or non-biological samples associated with the administrator account, and detecting selection of an affordance (e.g., admin tab affordance 2104) for displaying a user interface 2102 for the administrator account. In some embodiments, the user interface for the administrator account comprises a dashboard 2108, including a plurality of affordances for accessing sample reports (e.g., affordance 2114), test profiles (e.g., affordance 2116), users (e.g., affordance 2118), groups (e.g., affordance 2120), emails (e.g., affordance 2122), and/or settings (e.g., affordance 2124). FIG. 21 illustrates a customizable user interface comprising a dashboard for an administrator account, in accordance with some embodiments of the present disclosure.

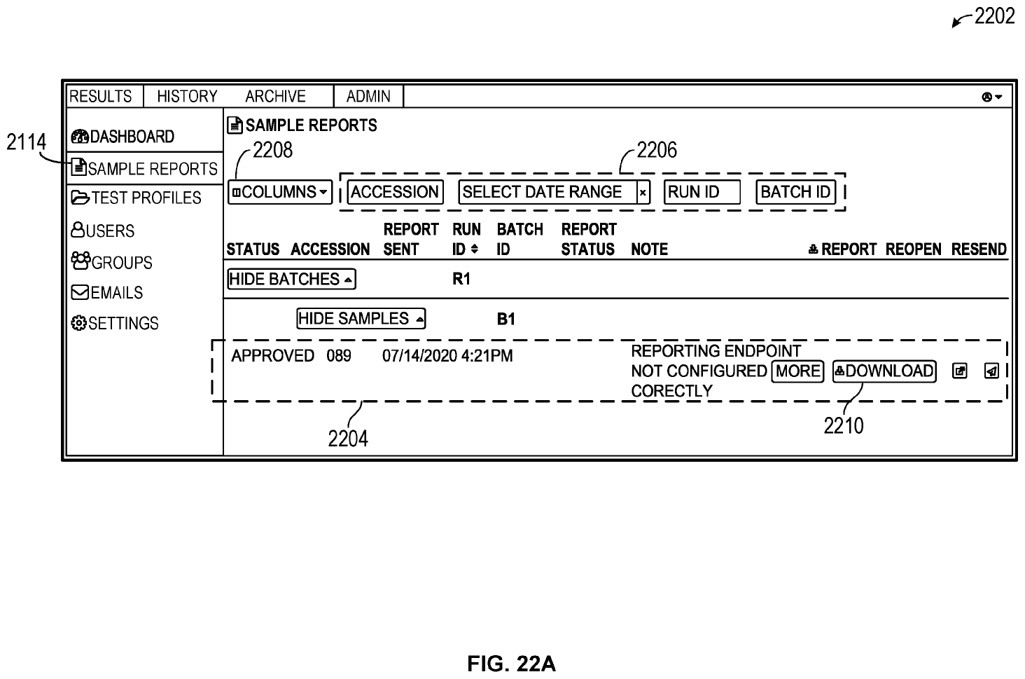

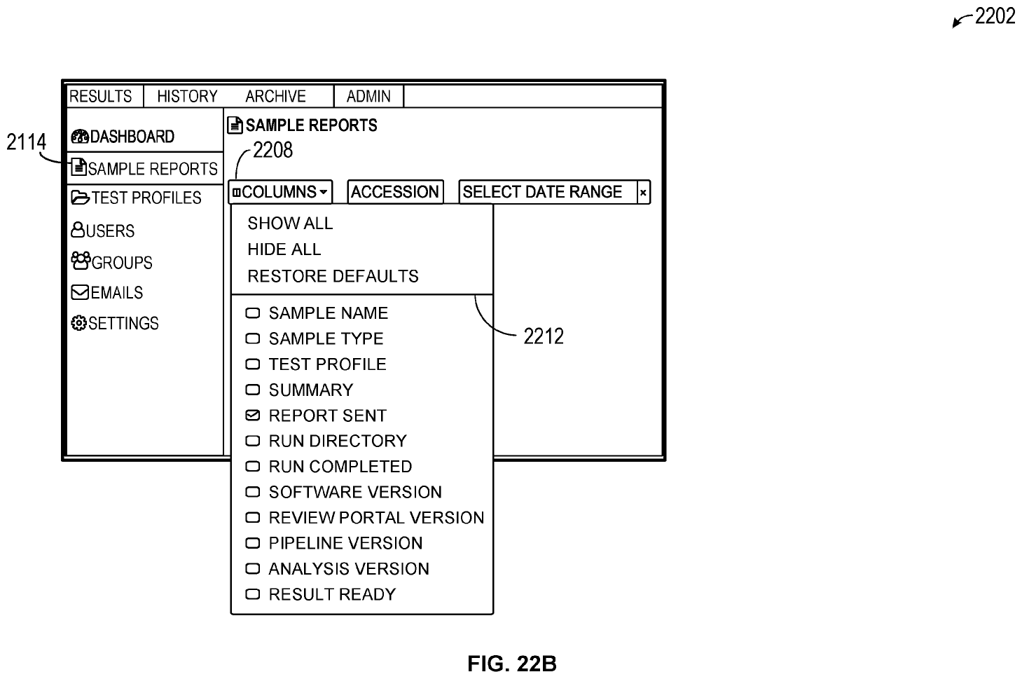

Sample reports. In some embodiments, the method further comprises, upon detecting a selection of the affordance for accessing sample reports 2114, displaying a user interface for sample reports 2202 comprising an index of sample reports 2204. In some embodiments, the user interface for sample reports 2202 comprises a plurality of features 2206 for searching, filtering, and/or sorting the index of sample reports. In some embodiments, the user interface for sample reports comprises an affordance for customizing the user interface 2208 (e.g., by selecting the plurality of features to be displayed on the user interface). In some embodiments, the user interface for sample reports 2202 comprises, for each sample report in the index of sample reports 2204, a summary of the sample report. In some embodiments, the user interface for sample reports 2202 comprises, for each sample report in the index of sample reports, an affordance 2210 for downloading the report, sending the report, opening the report, and/or expanding upon the summary of the sample report. FIGS. 22A and 22B illustrate an example customizable user interface 2202 comprising an index of sample reports 2204, in accordance with some embodiments of the present disclosure. For example, selection of the affordance 2208 for customizing the user interface for sample reports 2202 displays a dropdown list 2212 including a plurality of features for display. In some embodiments, features can be selected or deselected for display. In some embodiments, features include, but are not limited to, sample name, sample type, test profile, summary, report sent, run directory, run completed. analysis software version. review portal version, pipeline version, analysis version, and/or result ready.

Test profiles. As illustrated in FIG. 23A, in some embodiments, the method further comprises, upon detecting a selection of the affordance 2116 for accessing test profiles, displaying a user interface for test profiles 2302 comprising an index of test profiles 2310 (e.g., test profiles 2312-1, 2312-2, etc.). For instance, in some embodiments, the first customizable diagnostic template is stored as a test profile, such that the (i) subset of the plurality of sequencing statistics, (ii) subset of the set of at least 3 microorganisms, and (iii) subset of the plurality of mapping statistics specified by the first customizable diagnostic template are specified by one or more parameters saved in the test profile. In some embodiments, the second customizable diagnostic template is stored as a test profile. For instance, in some such embodiments, the second customizable diagnostic template comprises one or more filters to be applied to a result set, and the one or more filters are stored within a test profile. In some embodiments, the user interface for test profiles 2302 comprises a plurality of features for searching, filtering, and/or sorting the index of test profiles 2318. In some embodiments, the user interface for test profiles comprises an affordance for customizing the user interface (e.g., by selecting the plurality of features to be displayed on the user interface). In some embodiments, the user interface for test profiles 2302 comprises, for each test profile 2312 in the index of test profiles 2310, a summary of the test profiles. In some embodiments, the user interface for test profiles 2302 comprises an affordance 2314 for adding a new test profile to the index of test profiles. In some implementations, selection of the affordance 2314 displays a new display window for adding a new test profile (e.g., New Profile display window 2320), as illustrated in FIG. 23B. In some such implementations, the display window 2320 for adding a new test profile comprises one or more affordances for determining a profile name, a study description, a report type. a disease area, a read count normalization value, a retention status for non-profile organisms, a retention status for undetected organisms, a grouping status, and/or an annotation.

In some embodiments, the user interface for test profiles 2302 comprises, for each test profile 2312 in the index of test profiles 2310, an affordance 2316 for expanding upon the summary of the test profile. For example, FIG. 23C illustrates selection of an affordance 2316 (“View Details”) for expanding upon the summary of the test profile 2312-1 (“Respiratory Tract Infections (Validated)”), which causes display of an expanded test profile 2322 including profile information (e.g., profile name, study description, report type, disease area, etc.), report metadata (e.g., affordance 2305), relevant subclasses 2306 (e.g., viral, phage, plant virus, fungal virus, protist virus, endogenous virus, virophage, bacterial, fungal, parasite, viral AMR, bacterial AMR, fungal AMR, and/or parasite AMR), a run quality control metrics affordance 2324, a sample quality control metrics affordance 2326, and/or evidence categories 2308. Accordingly, in some embodiments, upon detection of a user selection of the affordance for expanding upon the summary of the test profile, the method further comprises displaying, on the display, an expanded test profile. In some embodiments, the expanded test profile 2322 comprises an affordance for viewing a plurality of organisms (e.g., microorganisms) included in the test profile. For instance, FIGS. 23C-D illustrates selection of an affordance 2328 (“View Organisms”) for viewing the identity of a plurality of organisms (e.g., microorganisms) included in the test profile, resulting in display of a test profile organism display 2332 that includes a list of the subset of microorganisms 402, in the set of microorganisms (e.g., at least 3, at least 5, or at least 10 microorganisms), specified by the respective test profile 2312-1 (“Respiratory Tract Infections (Validated)”). Accordingly, in some such embodiments, the first customizable diagnostic template is stored as a test profile, and the test profile organism display lists the subset of the set of microorganisms specified by the first customizable diagnostic template. For example, FIG. 23D illustrates a subset of microorganisms 402, including a first microorganism 402-tp-1 (e.g., Acetobacter indonesiensis), where, for each respective microorganism in the subset of microorganisms, the test profile organism display further includes a plurality of organism features. e.g., for example, organism name, reporting ID. class type, subclass, medical relevance, validation status, pathogen status, and/or one or more adjustable mapping statistics 2304 (e.g., RNA sensitivity, RNA specificity, DNA sensitivity, and/or DNA specificity)). In some embodiments, the test profile organism display 2332 further comprises an affordance for editing and/or deleting a respective organism entry in the list of microorganisms 402. In some embodiments, the test profile organism display 2332 further comprises an affordance 2336 for adding an organism to the plurality of organisms included in the test profile. Returning to FIG. 23C, in some embodiments, the expanded test profile 2322 further comprises an affordance 2330 for editing the test profile. In some embodiments, as depicted in FIG. 23E, selection of the affordance 2330 for editing the test profile displays a new display window for editing the test profile (e.g., Edit Profile display window 2338). In some such implementations, the display window 2338 for editing the test profile comprises one or more affordances for editing profile name, study description, report type, disease area, read count normalization value, retention status for non-profile organisms, retention status for undetected organisms, grouping status, and/or annotation. In some embodiments, as depicted in FIGS. 23F, 23G, and 23H, the selection of the affordances for run quality control metrics 2324 and sample quality control metrics 2326 in the expanded test profile 2322 further displays a plurality of run quality control metrics and/or sample quality control metrics (e.g., sequencing statistics 128-1, 128-2, 128-3, 128-K-1, 128-K-2, 128-K-3, 128-M-1, 128-M-2, 128-M-3, etc.). In some embodiments, e.g., as depicted in FIG. 23I, selection of the affordance for adding report metadata 2305 in the expanded test profile 2322 results in display of one or more text boxes for adding metadata to a final report (e.g., 2305-a). In some embodiments, e.g., as depicted in FIG. 23I, the expanded test profile 2322 further comprises one or more affordances for viewing, selecting, and/or deselecting a plurality of evidence categories 2308 (e.g., categories for evidence required for selective retention of features, upon application of the respective test profile to a result set).

In some embodiments, returning to FIG. 23A, the user interface for test profiles 2302 comprises, for each test profile 2312 in the index of test profiles 2310, an affordance for cloning (e.g., duplicating) the respective test profile. In some embodiments, the user interface for test profiles 2302 comprises, for each test profile 2312 in the index of test profiles 2310, an affordance for locking the respective test profile. In some embodiments, the user interface for test profiles 2302 comprises, for each test profile 2312 in the index of test profiles 2310, an affordance for deleting the respective test profile.

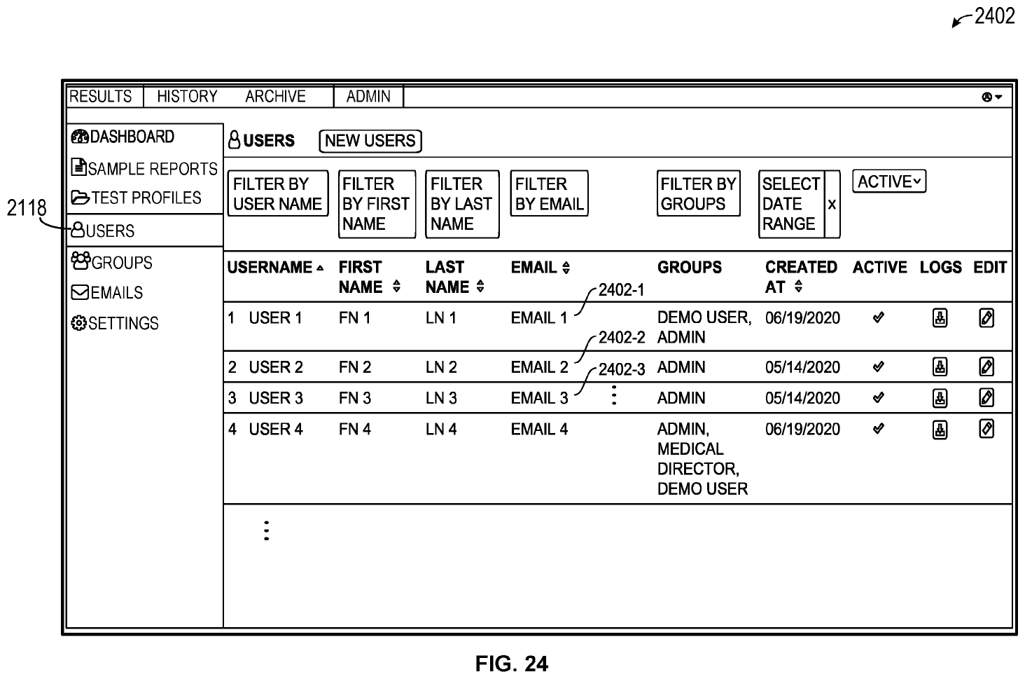

Users. As illustrated in FIG. 24, in some embodiments, the method further comprises, upon detecting a selection of the affordance 2118 for accessing users, displaying a user interface for users 2402 comprising a plurality of users 2402 (e.g., 2402-1, 2402-2, 2402-3, etc.) in an index of users. In some embodiments, the user interface for users comprises a plurality of features for searching, filtering, and/or sorting the index of users. In some embodiments, the user interface for users comprises an affordance for customizing the user interface (e.g., by selecting the plurality of features to be displayed on the user interface). In some embodiments, the user interface for users comprises, for each user in the index of users, a summary of the user information (e.g., groups, permissions, history logs, and/or email addresses). In some embodiments, the user interface for users comprises an affordance for adding a new user to the index of users. In some embodiments, the user interface for users comprises, for each user in the index of users, an affordance for editing, downloading, and/or expanding the information associated with the user.

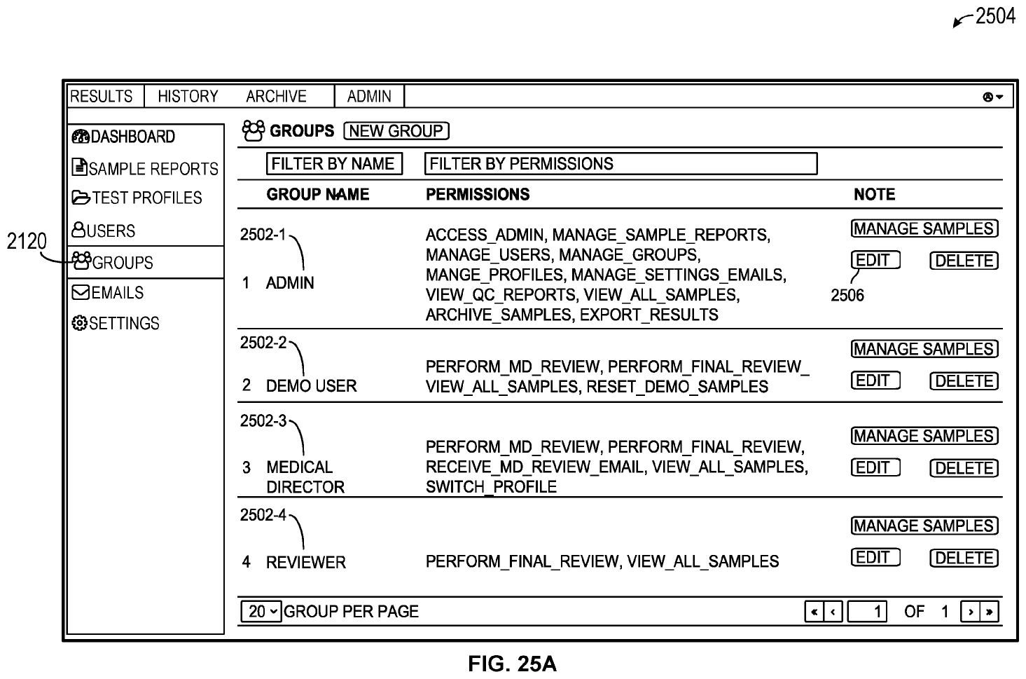

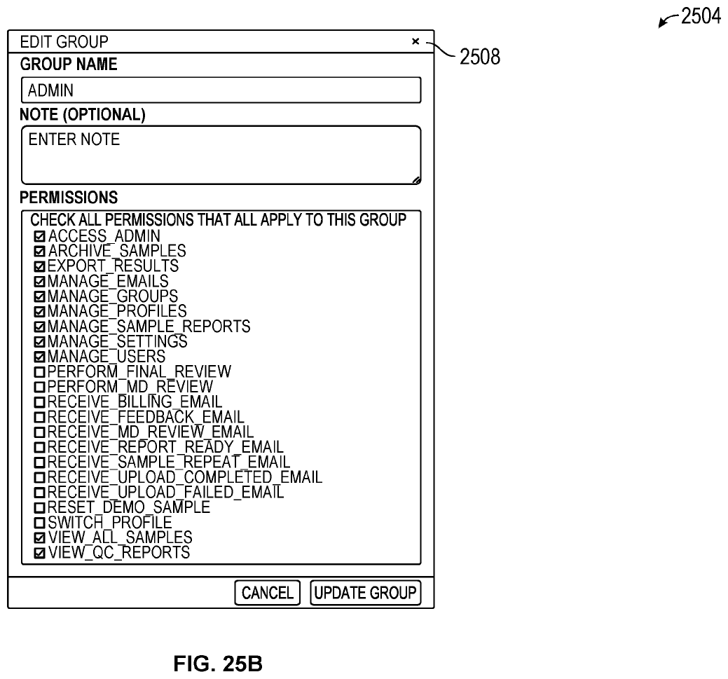

Groups and permissions. As illustrated in FIGS. 25A and 25B, in some embodiments, the method further comprises, upon detecting a selection of the affordance 2120 for accessing groups, displaying a user interface for groups 2504 comprising a plurality of groups 2502 (e.g., 2502-1, 2502-2, 2502-3, 2502-4, etc.) in an index of groups. In some embodiments, each respective group in the plurality of groups represents an account type (e.g., administrator account, demo account, medical director account, reviewer account, etc.). In some embodiments, a group comprises an access status for a user, including any permissions applied to the user upon membership into the group. In some embodiments, the user interface for groups comprises a plurality of features for searching, filtering, and/or sorting the index of groups. In some embodiments, the user interface for groups comprises an affordance for customizing the user interface (e.g., by selecting the plurality of features to be displayed on the user interface). In some embodiments, the user interface for groups comprises, for each group in the index of groups, a summary of the group information (e.g., permissions). In some embodiments, the user interface for groups comprises an affordance for adding a new group to the index of groups. In some embodiments, the user interface for groups comprises, for each group in the index of groups, an affordance for editing, managing, and/or expanding the information associated with the group. For instance, selection of an affordance 2506 for editing a respective group 2502-1 (e.g., administrator group) displays a new display window for editing the group (e.g., Edit Group display window 2508). In some such implementations, the display window 2508 for editing the group comprises one or more affordances for editing group name, notes, and/or permissions.

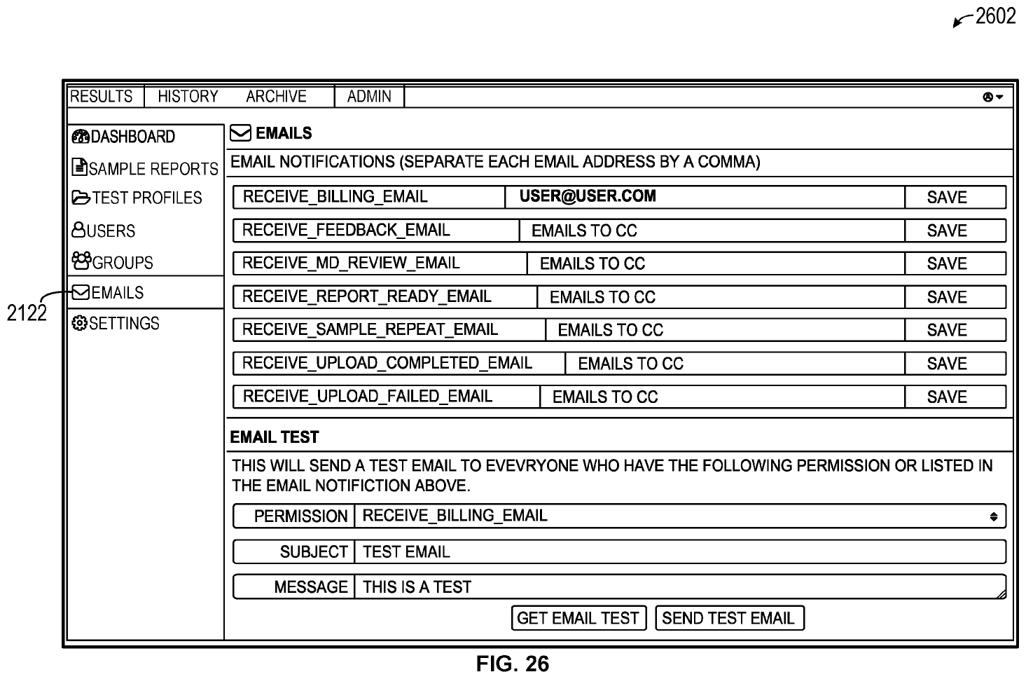

Emails. In some embodiments, as illustrated in FIG. 26, the method further comprises, upon detecting a selection of the affordance 2122 for accessing emails, displaying a user interface for emails 2602 comprising an index of groups of emails (e.g., a plurality of mailing lists). In some embodiments, a mailing list in the plurality of mailing lists is customized by adding, deleting, or editing an email in the respective mailing list. In some embodiments, the user interface for emails further comprises an affordance for composing and/or transmitting a message to a mailing list in the plurality of mailing lists.

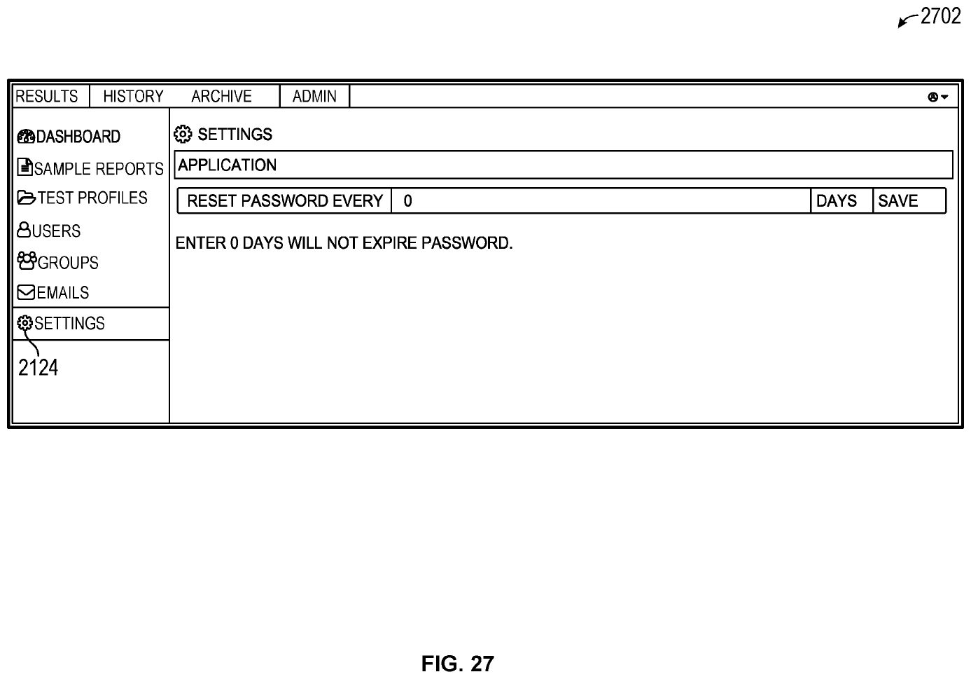

Settings. In some embodiments, as illustrated in FIG. 27, the method further comprises, upon detecting a selection of the affordance 2124 for accessing settings, displaying a user interface for settings 2702 comprising one or more features for managing a method for facilitating review of nucleic acid sequencing data prepared for identifying the presence of a subset of microorganisms and/or antimicrobial resistance markers in a biological or non-biological sample (e.g., from a subject), in accordance with some embodiments of the present disclosure.

In some embodiments, the displaying a user interface for the administrator account includes displaying an affordance for managing financial transactions (e.g., billing routes).

In some embodiments, the method further comprises, upon receiving a request to display an analysis of a result set obtained from a sequencing reaction of nucleic acids from the biological or non-biological sample, displaying, in the administrator account, any and/or all of the features described herein for reviewing, visualizing, and/or analyzing a result set for identifying the presence of a subset of microorganisms and/or antimicrobial resistance markers in a biological or non-biological sample.

Report Generation.

The systems and methods disclosed herein further include using the review and visualization tool to generate a report (e.g., a diagnostic report). FIGS. 17A-17H illustrate a report generated for an analysis of a result set, in accordance with some embodiments of the present disclosure.

For example, in some embodiments, the displaying, on the display, a customizable user interface (e.g., customizable user interface 401-2 in FIG. 16A), further comprises displaying a fifth affordance (e.g., export results affordance 1608) for exporting the analysis of the results set, thereby generating a report.

Referring to FIG. 2 at Block 210, the method further comprises generating a report 1702 including the summary of the subset of the plurality of sequencing statistics and, for each respective microorganism in the subset of the set of microorganisms satisfying a minimum mapping threshold in the result set, an identity of the respective microorganism and the summary of the subset of the plurality of mapping statistics for the respective nucleotide sequences in the plurality of nucleotide sequences mapped to the reference sequence for the respective microorganism.

In some embodiments, the report further comprises patient demographic information, a patient identifier, a pathogen identifier, and/or a non-pathogen identifier. In some embodiments, clinically or diagnostically relevant information is displayed on a first page of the report, and clinically or diagnostically irrelevant information is displayed on a second page of the report that is subsequent to the first page (e.g., in some embodiments, detected microorganisms that are classified as pathogens are displayed on an earlier page in the report than detected microorganisms that are not classified as pathogens. In some embodiments, report includes a description of sample type (e.g., DNA and/or RNA).

In some embodiments, the report further comprises a graphical representation of a mapping statistic in the subset of the plurality of mapping statistics. In some embodiments, the report further comprises a graphical representation of a sequencing statistic in the subset of the plurality of sequencing statistics. In some embodiments, the graphical representation is in the form of a heat map, a bar graph, and/or a table.

In some embodiments, the report further comprises a first therapeutic regimen based on the identity of a respective microorganism that satisfies a minimum mapping threshold in the result set (e.g., an identity of a detected microorganism).

For example, in some embodiments, a microorganism is reported if the microorganism is detected based on satisfaction of any parameter and/or filter described above, and/or any combination thereof as will be apparent to one skilled in the art. In some embodiments, a microorganism is reported if the microorganism is detected based on satisfaction of one or more parameters and/or filters included in the first customizable diagnostic template and/or the second customizable diagnostic template.

In some embodiments, the first therapeutic regimen is based on the classification of a respective microorganism as a pathogenic microorganism. In some such embodiments, the report further comprises a description of the pathogen. In some embodiments, the report further comprises an annotation of the pathogen based on clinical and/or health data. In some embodiments, the report further comprises a description of the first therapeutic regimen based on the pathogen. In some embodiments, the report further comprises an annotation of the first therapeutic regimen based on clinical and/or health data.

In some embodiments, the summary of the subset of the plurality of mapping statistics comprises an antimicrobial resistance status for a respective microorganism that satisfies a minimum mapping threshold in the result set, and the report further comprises a second therapeutic regimen based on the identity of the respective microorganism and the antimicrobial resistance status for the respective microorganism.

In some embodiments, the antimicrobial resistance status is based on the detection of an antimicrobial resistance gene in a detected microorganism. In some embodiments, the report further comprises a description of the antimicrobial resistance gene. In some embodiments, the report further comprises an annotation of the antimicrobial resistance gene based on clinical and/or health data.

In some embodiments, the report further comprises a patient response status. For example, in some embodiments, the report is generated to monitor a patient response to a treatment. In some embodiments, the report is generated to measure the efficacy of a treatment.

In some embodiments, the identity of the respective microorganism that is included in the report comprises an identity of two or more microorganisms in the set of microorganisms (e.g., the set of at least 3, at least 5, or at least 10 microorganisms) that share at least a threshold sequence identity in the respective reference sequences. For example, in some such embodiments, two or more microorganisms that are closely related (e.g., by sequence identity, class, parentage and/or phylogenetic lineage) will be included as detected in the report where the actual identity of the microorganism in the sample is ambiguous. In some embodiments, a parameter for determining when two or more microorganisms are reported in the case of ambiguous results is customized by user interaction (e.g., a cutoff threshold for reporting).

In some embodiments, the generating of a report comprises transmitting the report to a cloud computing infrastructure (e.g., an email).

In some embodiments, the report is generated as an email that can be sent to, for example, a patient, a medical practitioner (e.g., a primary physician), a hospital and/or a diagnostic laboratory.

In some embodiments, the method comprises generating an alert (e.g., an email) when the generation of the report is complete.

In some embodiments, the report is stored for retrieval. In some embodiments, the report is transmitted to a cloud computing infrastructure (e.g., a server) for storage.

In some embodiments, the method comprises generating an alert (e.g., an email) when transmission to the cloud computing infrastructure is complete.

In some embodiments, the report is exported in a printable format. In some embodiments, the report is generated as a printable document (e.g., a PDF).

Customization of Report.

As with the customization of the display, additional elements that can be customized include the specific parameters, metrics, and/or results to be included in the report (e.g., sequencing validation, sequencing statistics, mapping validation, mapping statistics, list of detected microorganisms, microbe-specific annotations, pathogen status, presence or absence of antimicrobial resistance genes, antimicrobial resistance gene annotations, and/or therapeutic treatments based on any of the above results or any combinations thereof).

Additional embodiments, substitutions, modifications, additions, deletions, and/or combinations of any of the systems and methods provided herein as possible, as will be apparent to one skilled in the art. See, for example, IDbyDNA, 2019, “Explify Software v1.5.0 User Manual,” Document No. TH-2019-200-006, pp. 1-44, which is hereby incorporated by reference herein in its entirety.

### Examples

In some embodiments, the systems and methods described herein are useful for a variety of applications including, but not limited to, metagenomics, cancer diagnostics, human variation (pharmacogenomics and ancestry), and agricultural and food analysis. In some embodiments, the systems and methods described herein are useful for bacterial and fungal classification, viral classification, parasite classification, human mRNA transcript profiling, identification of infection and contamination, and/or detection of microorganisms for, e.g., education, consumers, food safety and authenticity, hospital safety and contamination monitoring, biological product quality and safety monitoring, animal disease diagnostics and treatment, microbial strain profiling, tumor profiling, forensic profiling, and/or genetic testing.

### Example 1—Explify Review Portal

In some embodiments, information about a sample, such as information regarding entities associated with the sample, are presented using a software program or platform. The software platform can include one or more components, such as a component for providing information about a sample, a component for analyzing sequencing information (e.g., performing a k-mer based analysis). a component for analyzing and classifying processed sequencing reads, and a component for supporting laboratory sample preparation. The Explify Software Platform (e.g., Software v1.5.0) is an example of a software platform that includes three such components: the Explify ReviewPortal, which is a web browser-accessible dashboard application; the Explify Analysis Pipeline, which processes raw NGS data for analysis by the Explify Classification Algorithm: and the Explify SeqPortal web-based application (also called Workflow Manager), which supports sample information entry and laboratory sample preparation.

The ReviewPortal component of the Explify Software Platform is a web application for laboratory users. The Explify Analysis Pipeline analyzes the results of a sequencing run to report the detection of pathogens. Review Portal users review these detection calls and verify their validity. The decisions made by users of the Review Portal are used to generate reports. The Review Portal enforces a workflow to ensure the integrity of detection decisions. Each sequencing run contains up to eight samples: a positive external control, a negative external control, and up to six test samples. Both controls are reviewed before the test samples, in case the controls indicate a problem that would lead to incorrect results. Every sample is reviewed by at least two laboratory reviewers and a senior reviewer. A senior reviewer has access to additional metrics that will aid in making detection decisions. When a test sample has undergone all necessary stages of review, it is ready for Final Review. A Final Reviewer reviews the detection decisions made on a sample and submits the final report. Based on sequencing quality metrics and the results of the external controls, the Result Review SOP may require that sequencing be repeated on a sample or run. A reviewer may mark a sample or run for repeat, which will disable review of the sample or run. Once repeated sequencing results are processed by the Analysis Pipeline, the review will be re-enabled with updated results. The updated results on test samples are displayed alongside the original results.

See, for example, IDbyDNA, 2019, “Explify Software v1.5.0 User Manual,” Document No. TH-2019-200-006, pp. 1-44, which is hereby incorporated by reference herein in its entirety.

Example illustrations of a system and method for facilitating review of nucleic acid sequencing data, in accordance with an embodiment of the present disclosure (e.g., the ReviewPortal component of the Explify Software Platform), are described below, with reference to FIGS. 3-28.

FIG. 3A illustrates an example of a results dashboard 302 displaying a list of samples 304 (e.g., 304-1, 304-2, 304-3) in a pending sample queue 306, in accordance with some embodiments of the present disclosure. Selection of “Show/Hide Batches” affordance 308 expands or contracts the pending sample queue 306 to show or hide a plurality of sample batches within a respective sample run. Selection of affordance 308 to “Show Batches” displays a “Show/Hide Samples” affordance 310 for expanding or contracting the pending sample queue 306 to show or hide a plurality of samples within a respective batch.

Each sample 304 in the list of samples comprises a plurality of features, including a review status 312 (e.g., MD Review, Final Review, etc.) and a summary 318, where each summary includes an indication of a sequencing statistic (e.g., a run quality control metric 320 and/or a sample quality control metric 322), and an indication of a mapping statistic (e.g., a type of microorganism 324 and/or an AMR gene detected in the sample). A search function can be performed using manual entry boxes 330 (e.g., 330-1, 330-2, 330-3, etc.), which can be used to filter the plurality of samples by searching for a value or a text-string in any desired feature of the sample, such as a sample accession number, sample type, run identifier, batch identifier, and/or date range. Additional features for each sample can be displayed (and/or made searchable) using an affordance 326. For example, as illustrated in FIG. 3B. selection of affordance 326 expands a dropdown list 328, from which any of the additional features can be selected or deselected (e.g., using checkboxes) for viewing in results dashboard 302.

Returning to FIGS. 3A, selection of a sample 304-1 (e.g., sample number 5958) using “Review” affordance 332 generates a request to display an analysis of a result set for the sample 304-1. The displayed analysis is illustrated in FIGS. 4, 6G, and 11 as customizable user interface 401-1 and generally includes (i) a review status 440, (ii) a first affordance 604 for updating the review status 440, (iii) a summary of a subset of sequencing statistics 420 (e.g., 420-1, 420-2, 420-3), (iv) for each respective microorganism 402 (e.g., 402-1) satisfying a minimum mapping threshold in the result set, a corresponding summary of a subset of mapping statistics (e.g., 404-418, 424-428), and (v) a second affordance 1104 for applying a filter to the analysis. In particular, as illustrated in FIG. 4, the displayed analysis for sample 304-1 indicates a subset of detected microorganisms (“B: Bacteria”), including Escherichia coli 402-1, and/or detected AMR genes (“B: Bacterial AMR”), including ampC 422-1.

Metadata for the sample is displayed as a header 438 in the user interface 401-1, and a result summary 452 indicates a status of the analysis of the result set (“inconclusive”) and a status of an analytical sensitivity (“adequate”). A review status 440 for the nucleic acid sequencing data indicates a current review status 440-1 (“MD”) and a next following review status 440-2 (“Final”). Submission of the current review updates the review status 440 from the current review status to the next following review status and can be performed using a review action 450. For instance, as illustrated in FIG. 6G, selection of a “Submit Review” affordance 604 updates the review status by submitting the current review. Other review actions 450 includes a “Reset Review” affordance 606 and a “Cancel Review” affordance 608.

Returning to FIG. 4, the displayed analysis for sample 304-1 (e.g., in customizable user interface 401-1) includes a summary of the detected microorganism Escherichia coli 402-1, where the summary comprises a plurality of organism features including at least an alert status 424 (e.g., N: no call: A: alert; C: critical), a pathogen status 426, an organism name 428, evidence 404, sample type 406, percent coverage 408, ANI 410, median depth 412, read count 414, quantity 416, reference length 418, and/or other sequencing statistics and/or mapping statistics.

Selection of a “Show” affordance 434 or clicking on the entry for the microorganism generates a new display window 502 overlaid on the customizable user interface 401-1, which provides an expanded summary for the microorganism 402-1. For instance, as illustrated in FIGS. 5A and 5B, graphical representations of a mapping statistic (e.g., fold coverage versus nucleotide position) are shown for RNA (left panels 504-1) and DNA (right panels 504-2) alignments. Graphical representations can be toggled between linear scale (FIG. 5A) and log scale (FIG. 5B) using a “Linear” affordance (e.g., 506-A-1, 506-A-2) and/or a “Log” affordance (e.g., 506-B-1, 506-B-2) in the display window 502. The expanded summary for the microorganism further includes some of all of the plurality of organism features and/or additional features such as percent coverage cutoff 409.

Referring to FIG. 5A, the display window 502 also includes a commenting affordance 508 for adding, editing, submitting and/or removing a comment for the microorganism 402-1 using a comment window 510. As illustrated in FIGS. 5C and 5D, selection of affordance 508-1 displays a comment window 510-2 for appending a comment to an RNA alignment, and selection of affordance 508-2 displays a comment window 510-1 for appending a comment to a DNA alignment. The display window 502 further includes a “Copy and Blast” affordance 512 for accessing a reference sequence database (e.g., BLAST, NCBI, etc.) and performing a nucleic acid sequence comparison using the reference sequence (e.g., genome) for the microorganism 402-1. The display window 502 further includes a validation affordance 514 for validating and/or displaying a validation status of the subset of sequencing statistics and/or the subset of mapping statistics for the microorganism 402-1.

Returning to FIG. 4, the displayed analysis for sample 304-1 (e.g., in customizable user interface 401-1) includes a summary of the detected bacterial AMR gene ampC 422-1, where the summary comprises a plurality of AMR gene features including, for instance, a bit score 430 and PID 432. Selection of a “Show” affordance 436 provides a new display window 602 overlaid on the customizable user interface 401-1, which provides an expanded summary for the AMR gene 422-1. For instance, as illustrated in FIG. 6A. text-based and/or graphical representations (e.g., in togglable linear and/or log scale) of mapping statistics are displayed for RNA (left panels 614-1) and DNA (right panels 614-2) alignments.

The display window 602 further includes a “Copy and Blast” affordance 628 for accessing a reference sequence database (e.g., BLAST, NCBI, etc.) and performing a nucleic acid sequence comparison using a nucleic acid sequence for the AMR gene 422-1.

As shown in FIGS. 6A and B, the display window 602 includes an internal notes affordance 630 for displaying, adding, editing, submitting and/or removing an internal note associated with the AMR gene 422-1. For instance, selection of the internal notes affordance 630 displays an internal notes window 624 for appending a comment to an RNA alignment. As shown in FIGS. 6A and C, the display window 602 also includes a commenting affordance 622 (e.g., 622-1, 622-2) for displaying, adding, editing, submitting and/or removing a comment for the AMR gene 422-1. For instance, selection of the commenting affordance 622-1 displays a comment window 626 for appending a comment to an RNA alignment.

As illustrated in FIGS. 6D-F, the summary of the detected bacterial AMR gene ampC 422-1 in customizable user interface 401-1 includes additional affordances for displaying information associated with the AMR gene, including an antibiotic affordance 444 for displaying an antibiotic annotation window 616 (e.g., including one or more of a therapeutic treatments and/or drug classes associated with the gene), an associated organism affordance 446 for displaying a microorganism window 618 (e.g., including one or more microorganisms associated with the gene), and/or an information affordance 448 for displaying an aggregated information window 620 (e.g., including expanded feature information for the AMR gene).

FIGS. 7, 8, and 9 illustrate examples of control samples, including a positive control sample 304-cp, a negative control sample 304-cn, and a blank control sample 304-blk. As illustrated in FIG. 7, a display for the analysis 401-cp of the positive control sample 304-cp is characterized by robust detection of a plurality of microorganisms 402 (e.g., 402-1, 402-2, 402-3, etc.) exhibiting a high percentage of coverage 408 and average nucleotide identity (ANI) 410 for the reference genomes of detected microorganisms detected in the positive control. In contrast, FIGS. 8 and 9 illustrate failure to detect any microorganisms in either the negative control sample 802 (e.g., in display 401-cn) or the blank control sample 902 (e.g., in display 401-blk). Notably, passing scores for quality control checks at the sample (420-3), batch (420-2), and run level (420-1) (e.g., represented by check marks) indicated that the sequencing and mapping processing prior to microorganism detection analysis were performed successfully, providing an additional layer of confidence in the analysis of the control sample result sets.

Toggling between one or more samples (e.g., 304-1, 304-2, 304-cp, 304cn, and/or 304-blk), batches, and/or runs can be performed, as illustrated in FIG. 10, using a sample selection affordance 1002 (e.g., a dropdown list 1006). Sample selection affordance 1002 can be accessed from within the display 401 of any sample 304, such as display 401-2 of sample 304-2 (e.g., sample number 6011A). The display for each respective sample in a plurality of samples can include any of the features and/or embodiments for any other sample, including a corresponding (i) respective review status, (ii) a first affordance for updating the review status, (iii) a respective summary of a subset of sequencing statistics, (iv) for each respective microorganism in a subset of microorganisms satisfying a minimum mapping threshold in the respective result set, a corresponding summary of a subset of mapping statistics, and (v) a second affordance for applying a filter to the result set. Accordingly, as illustrated in FIGS. 4 and 10, customizable user interfaces 401-1 and 401-2 each display a unique respective subset of microorganisms (e.g., 402-1 and 402-4) and a unique respective subset of AMR genes (e.g., 422-1 and 422-2) identified by their respective analysis.

Referring again to FIG. 4, the customizable user interface 401-1 includes a “Show All” affordance for displaying all of the microorganisms 402 in a set of microorganisms (e.g., at least 3, at least 5, or at least 10 microorganisms). FIG. 11 illustrates that selection of “Show All” affordance 442 expands the customizable user interface 401-1 to display an expanded list of microorganisms 402 (e.g., all of the microorganisms in a set of at least 3, at least 5. or at least 10 microorganisms). Each respective microorganism entry in the expanded list of microorganisms includes a summary of the respective microorganism, as described above with reference to FIG. 4. Additionally, as illustrated in FIG. 11, in some implementations, the customizable user interface 401-1 includes a “Filter” affordance 1104 for displaying (or, alternately, hiding) one or more filters that can be applied to (i) the subset of sequencing statistics, (ii) the subset of microorganisms, and/or (iii) for each respective microorganism in the subset of microorganisms, the corresponding subset of mapping statistics. Selection of the “Filter” affordance displays one or more filters 1204, including, but not limited to, a “Medically Relevant” filter 1208, an “Evidence” filter 1206, a “Phylogenetic Lineage” filter 1210, an “Organism Name” filter 1212, an “RNA” filter 1214, and/or a “DNA” filter 1216.

In some implementations, the customizable user interface 401 includes various affordances for accessing and/or visualizing the features of a sample 304, a microorganism 402, and/or an AMR gene 422. As illustrated in FIGS. 6G, 13 and 15, a wide range of user interactions can be performed to display expanded feature information. For instance, hovering a pointer (e.g., a cursor) over bit score value 612 displays an overlay display 610. Similarly, hovering a pointer over a percent coverage value displays an overlay display 1304 of the cutoff threshold for detection for percent coverage. The overlay display is removed when the pointer is moved away from the respective feature. Various affordances such as checkboxes, manual text entry boxes, and dropdown lists can be employed for entry and/or selection of one or more features, such as dropdown list 1302 for updating an alert status for a respective microorganism and/or checkbox 1512 for selecting a pathogen status. Any one or more features can also be represented by a variety of visual indicators distinguishable by color, symbol, and/or shade, as shown by review status visual indicators 1504 in FIG. 15D.

Organisms can be added to the analysis during the review phase (e.g., upon display of the analysis of the result set). For instance, referring to FIG. 14, an example customizable user interface 401-2 includes an “Add Organism Form” affordance 1402 for adding a microorganism to the subset of the set of microorganisms. The “Add Organism Form” affordance 1402 can include affordances for entry of, for example, an organism name 1404, a detection status 1408, a category 1410, a validation status 1406, an alert 1414, an abundance status 1424, a class type 1412, a number of RNA reads 1416, an RNA reference length 1420, a number of DNA reads 1418, a DNA reference length 1422, a report comment 1426, and/or an internal note 1428. The “Add Organism Form” affordance 1402 can further include an “Add Organism” affordance 1430 for finalizing and submitting the added organism to the subset of the set of microorganisms.

Referring again to FIGS. 4 and 10, the result summary 452 displayed in customizable user interface 401-1 or 401-2 can be modified using, e.g., an edit result summary affordance. For example, affordance 1004 in FIG. 10 provides an example of an “Edit Result Summary” affordance. Selection of the affordance 1004 generates a new display window 1502 (e.g., “Edit Result Summary” window 1502 in FIG. 15A) including one or more affordances for modifying the result summary 1508, modifying the analytical sensitivity 1510, and/or adding a comment 1506. Selection of affordance 1508 displays a dropdown list of result summary options, as illustrated in FIG. 15B. Selection of affordance 1510 displays a dropdown list of analytical sensitivity options, as illustrated in FIG. 15C.

In some implementations, the customizable user interface 401 includes one or more affordances for performing reporting actions. FIG. 16A illustrates an example affordance 1602 in customizable user interface 401-2 for selecting from a plurality of reporting actions, including an “Export Results” affordance 1608, a “Preview” affordance 1606, and a “Report” affordance 1604. Additional affordances include a “Sample” affordance 1610 and a “Batch” affordance 1612 for selecting the features to be included in the exported results, the preview, and/or the report. Accordingly, selection of “Export Results” affordance 1608 displays an “Export Results” display window, including a plurality of features that can be selected or deselected for inclusion in the exported results and/or the report. As illustrated in FIG. 16B, a plurality of features can be selected or deselected for a respective sample 1614, a respective organism 1616, and/or for a respective organism sheet 1618. An example report 1702, generated via selection of the “Report” affordance 1604, is provided in FIGS. 17A-H.

Returning again to FIG. 4, the displayed analysis for sample 304-1 in customizable user interface 401-1 indicates quality control checks at the sample (420-3), batch (420-2), and run level (420-1). Correspondingly, affordances 1902, 2002, and 1802 can be selected to expand upon the sample (e.g., 1904-1, 1904-2, 1904-3, . . . , 1904-M. etc.), batch (e.g., 2004-1, 2004-2, etc.), and run-level (e.g., 1804-1, 1804-2, 1804-3, etc.) quality control metrics, respectively. Expanded displays for quality control metrics upon selection of affordances 1802, 1902, and 2002 are illustrated in FIGS. 18A-B, 19A-B, and 20A-B, respectively. For instance, chart views 1806 and table views 1808 of quality control data (e.g., 1812, 1906, 1908 are illustrated, the display of which can be toggled by various affordances (e.g., 1810, 1814, 1910).

Another feature of the present example (e.g., the ReviewPortal) includes an administrator access feature. FIG. 21 illustrates a customizable user interface comprising a user interface 2102 for an administrator account, in accordance with some embodiments of the present disclosure. The administrator user interface 2102 includes a plurality of tabs, comprising at least a “Results” affordance 2106, selection of which displays an index of biological or non-biological samples 306 (e.g., in a results dashboard 302), a “History” affordance 2110, selection of which displays a second index of biological or non-biological samples (e.g., index 2802 illustrated in FIG. 28), an “Archive” affordance 2112, and an “Admin” affordance 2104. Selection of “Admin” affordance 2104 displays an instance of the administrator user interface 2102 including a plurality of affordances for accessing a dashboard (e.g., affordance 2108), sample reports (e.g., affordance 2114), test profiles (e.g., affordance 2116), users (e.g., affordance 2118), groups (e.g., affordance 2120), emails (e.g., affordance 2122), and/or settings (e.g., affordance 2124).

Upon detecting a selection of “Sample Reports” affordance 2114, a user interface for sample reports 2202 comprising an index of sample reports 2204 is displayed, as illustrated in FIG. 22A. Search, filter, sort, and/or customization functions can be performed for the index of sample reports using various affordances 2206 and 2208, for instance by searching and/or selecting for one or more features in a list of features 2212 (see, e.g., FIG. 22B). An affordance 2210 can also be used for downloading the report, sending the report, opening the report, and/or expanding upon the summary of the sample report.

As illustrated in FIG. 23A, upon detecting a selection of “Test Profiles” affordance 2116, a user interface for test profiles 2302 comprising an index of test profiles 2310 (e.g., test profiles 2312-1, 2312-2, etc.) is displayed. Search, filter, sort, and/or customization functions can be performed for the index of test profiles using various affordances 2318. New test profiles can be added using “New Profile” affordance 2314 via New Profile display window 2320 (see, e.g., FIG. 23B).

As illustrated in FIGS. 23A and C. existing test profiles 2312 can be expanded upon using “View Details” affordance 2316. For instance, selection of “View Details” affordance 2316 expands upon example test profile 2312-1 (“Respiratory Tract Infections (Validated)”), thus displaying an expanded test profile 2322 including profile information, report metadata (e.g., affordance 2305), relevant subclasses 2306, a run quality control metrics affordance 2324. a sample quality control metrics affordance 2326, and/or evidence categories 2308. As depicted in FIGS. 23C-D, selection of an affordance 2328 (“View Organisms”) displays a list of a plurality of organisms (e.g., microorganisms 402) included in the test profile, along with one or more corresponding mapping statistics 2304 and an option to add additional organisms 2336, in a test profile organism display 2332. Returning to FIG. 23C, the expanded test profile 2322 further includes an “Edit” affordance 2330 for editing the test profile. As depicted in FIG. 23E, selection of the “Edit” affordance 2330 displays an “Edit Profile” display window 2338 including various affordances for manual entry and/or feature selection. Selection of additional affordances in the expanded test profile 2322 provides further information on run quality control metrics 2324, sample quality control metrics 2326, report metadata 2305, and evidence categories 2308, as illustrated in FIGS. 23F-1.

Returning to FIG. 21, selection of the “Users” affordance 2118 displays a “Users” window 2402 comprising a plurality of users (2402-1, 2402-2, 2402-3) in an index of users (see, e.g., FIG. 24). Selection of the “Groups” affordance 2120 displays a “Groups” window 2504 comprising a plurality of groups 2502 in an index of groups (see, e.g., FIGS. 25A and 25B). Group permissions and other details can be edited using an “Edit Group” window 2508 accessed via “Edit” affordance 2506. Selection of the “Emails” affordance 2122 displays an “Emails” window 2602 comprising an index of groups of emails (see, e.g., FIG. 26). Selection of the “Settings” affordance 2124 displays a “Settings” window 2702 comprising one or more features for managing a method for facilitating review of nucleic acid sequencing data (see, e.g., FIG. 27).

Various elements described in the present example are disclosed in greater detail in the above sections. Accordingly, the example system and method described with reference to FIGS. 3-28 (e.g., the ReviewPortal) is not intended to be limiting but is further contemplated for use with any of the embodiments disclosed in the above sections, as well as any substitutions, modifications, additions, deletions, and/or combinations thereof as will be apparent to one skilled in the art.

### Example 2: Example Workflow

FIG. 29 illustrates an example workflow for processing biological or non-biological samples for analysis of presence of microorganisms, in accordance with some embodiments of the present disclosure. In Block 2900, samples are collected (e.g., as described herein). Samples may be collected from biological or non-biological sources including human subjects, environmental sources, industrial sources, or other sources. Samples may include fluids and/or solids. Samples may be processed to prepare the samples for subsequent sequencing (2910). Samples may optionally be divided into two or more portions for subsequent analysis. Samples that will be analyzed for nucleic acids included therein may be process and/or analyzed separately from samples that will be analyzed for polypeptides included therein. Sequences of nucleic acid molecules and/or polypeptides of the sample may be analyzed using nucleic acid and/or polypeptide sequencing techniques (2920 and 2930). Data prepared from this analysis, including sequencing reads, may be collected and optionally combined. Data may be stored locally and/or in a web- or cloud-based storage system. Data may be compared against sequences in one or more reference databases (e.g., as described herein) (2940). Data may be processed and interpreted using a software program, such as a web-based software program. A user may prepare and/or interpret various representations of the data. The data may be analyzed to interpret the nucleic acid molecules and/or polypeptides included in the sample, thereby identifying microorganisms, viruses, genes, or other contents of the sample (2950). A variety of representations of the data may be prepared (e.g., as described herein). Such representations and reports may be used to inform a variety of interventions including medical interventions and physical interventions (e.g., as described herein). For example, a report may be used to inform a treatment regimen for a patient.

### CONCLUSION

All references cited herein are incorporated herein by reference in their entirety and for all purposes to the same extent as if each individual publication or patent or patent application was specifically and individually indicated to be incorporated by reference in its entirety for all purposes.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation. has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A method for facilitating review of nucleic acid sequencing data prepared for identifying the presence of a subset of microorganisms and/or antimicrobial resistance markers in a first biological or non-biological sample, the method comprising:
at a computer system having a display, one or more processors, and memory storing one or more programs for execution by the one or more processors;
A) receiving a request to display an analysis of a result set obtained from a sequencing reaction of nucleic acids from the first biological or non-biological sample, wherein the result set includes (i) a plurality of sequencing statistics from the sequencing reaction, (ii) a plurality of nucleotide sequences mapped against a plurality of reference sequences corresponding to a set of at least 3 microorganisms, and (iii) for each respective microorganism in the set of at least 3 microorganisms, a corresponding plurality of mapping statistics for the mapping of respective nucleotide sequences to the reference sequence of the respective microorganism;
B) applying, responsive to the request, a first customizable diagnostic template to the result set, wherein the customizable diagnostic template specifies (i) a subset of the plurality of sequencing statistics, (ii) a subset of the set of at least 3 microorganisms, and (iii) a subset of the plurality of mapping statistics; and
C) displaying, on the display, a customizable user interface comprising:
(i) a review status for the nucleic acid sequencing data,
(ii) a first affordance for updating the review status for the nucleic acid sequencing data,
(iii) a summary of the subset of the plurality of sequencing statistics,
(iv) for each respective microorganism in the subset of the set of at least 3 microorganisms satisfying a minimum mapping threshold in the result set, a corresponding summary of the subset of the plurality of mapping statistics for the respective nucleotide sequences in the plurality of nucleotide sequences mapped to the reference sequence of the respective microorganism, and
(v) a second affordance for applying a second customizable diagnostic template to the result set.

2. The method of claim 1, wherein the customizable user interface further comprises:
(vi) a third affordance for expanding upon the corresponding summary of the subset of the plurality of sequencing statistics, and
(vii) a fourth affordance for expanding upon a summary of a plurality of values for the subset of the plurality of mapping statistics.

3. The method of claim 2, wherein upon user selection of the fourth affordance, the method further comprises displaying, on the display, an affordance for amending the subset of the plurality of mapping statistics.

4. The method of claim 2 or 3, wherein upon user selection of the fourth affordance, the method further comprises displaying, on the display, an affordance for validating the subset of the plurality of mapping statistics.

5. The method of any one of claims 2-4, wherein upon user selection of the fourth affordance, the method further comprises displaying, on the display, an affordance for appending a user-inputted text string to the subset of the plurality of mapping statistics.

6. The method of any one of claims 2-5, wherein upon user selection of the fourth affordance, the method further comprises displaying, on the display, an affordance for accessing a reference sequence database comprising at least the reference sequence for the respective microorganism.

7. The method of any one of claims 2-6, wherein upon user selection of the fourth affordance, the method further comprises displaying, on the display, a graphical representation of a mapping statistic in the subset of the plurality of mapping statistics.

8. The method of any one of claims 2-7, wherein upon user selection of the fourth affordance, the method further comprises displaying, on the display, a graphical representation of a sequencing statistic in the subset of the plurality of sequencing statistics.

9. The method of claim 7 or 8, wherein the graphical representation is in the form of a heatmap.

10. The method of any one of claims 1-9, wherein the customizable user interface further comprises a fifth affordance for exporting the analysis of the results set, thereby generating a report.

11. The method of any one of claims 1-10, the method further comprising:
D) generating a report including:
the summary of the subset of the plurality of sequencing statistics and, for each respective microorganism in the subset of the set of at least 3 microorganisms satisfying a minimum mapping threshold in the result set, an identity of the respective microorganism and the summary of the subset of the plurality of mapping statistics for the respective nucleotide sequences in the plurality of nucleotide sequences mapped to the reference sequence for the respective microorganism.

12. The method of claim 11, wherein the report further comprises a graphical representation of a mapping statistic in the subset of the plurality of mapping statistics.

13. The method of claim 12, wherein the graphical representation is in the form of a heat map.

14. The method of any one of claims 11-13, wherein the report further comprises a first therapeutic regimen based on the identity of a respective microorganism that satisfies a minimum mapping threshold in the result set.

15. The method of any one of claims 11-14, wherein the summary of the subset of the plurality of mapping statistics comprises an antimicrobial resistance status for a respective microorganism that satisfies a minimum mapping threshold in the result set, and the report further comprises a second therapeutic regimen based on the identity of the respective microorganism and the antimicrobial resistance status for the respective microorganism.

16. The method of any one of claims 11-15, wherein the report further comprises a patient response status.

17. The method of any one of claims 11-16, wherein the identity of the respective microorganism comprises an identity of two or more microorganisms in the set of at least 3 microorganisms that share at least a threshold sequence identity in the respective reference sequences.

18. The method of any one of claims 11-17, wherein the generating a report comprises transmitting the report to a cloud computing infrastructure.

19. The method of any one of claims 1-18, wherein upon user selection of the second affordance, the method further comprises applying the second customizable diagnostic template to the result set by applying a filter to the subset of the plurality of sequencing statistics, the subset of the set of at least 3 microorganisms, and the subset of the plurality of mapping statistics.

20. The method of claim 19, wherein the second customizable diagnostic template includes a disease condition filter,
a microorganism in the set of at least 3 microorganisms is annotated with the disease condition based on a threshold number of co-occurrences of the microorganism and the disease condition in a database, and
the applying the filter selectively retains one or more microorganisms annotated with the disease condition.

21. The method of claim 19 or 20, wherein the second customizable diagnostic template includes a target microorganism filter, and the applying the filter selectively retains one or more microorganisms that share at least a threshold sequence identity to the target microorganism.

22. The method of any one of claims 19-21, wherein
the second customizable diagnostic template includes an antimicrobial resistance filter,
the applying the filter selectively retains one or more microorganisms, and
the mapping of the respective nucleotide sequences in the plurality of nucleotide sequences to the reference sequence for the respective microorganism indicates the presence of an antimicrobial resistance marker.

23. The method of any one of claims 19-22, wherein the second customizable diagnostic template includes a mapping statistics filter, and the applying the filter selectively retains one or more microorganisms having at least a threshold value for a mapping statistic in the plurality of mapping statistics.

24. The method of any one of claims 19-23, wherein the second customizable diagnostic template includes one or more stored parameters specifying the filter, the subset of the set of at least 3 microorganisms, and the subset of the plurality of mapping statistics.

25. The method of any one of claims 1-24, wherein the review status is selected from the group consisting of a first review, a second review, a medical director review, a final review, passed, and an approved status.

26. The method of any one of claims 1-25, wherein the customizable user interface further comprises a result history comprising at least the first customizable diagnostic template applied to the result set, wherein the review status of the result set is approved.

27. The method of any one of claims 1-26, wherein the customizable user interface further comprises a result summary including a status of the analysis of the results set based on the plurality of mapping statistics for each respective microorganism in the set of at least 3 microorganisms, and wherein the status is selected from the group consisting of: invalid, inconclusive, microorganisms detected, microorganisms detected including potential pathogens, and no microorganisms detected.

28. The method of any one of claims 1-27, wherein the subset of the set of at least 3 microorganisms satisfying the minimum mapping threshold is a threshold number of microorganisms with the highest values for a percent sequence alignment, based on an alignment of respective nucleotide sequences to the reference sequence of the respective microorganism.

29. The method of any one of claims 1-28, wherein the subset of the set of at least 3 microorganisms satisfying the minimum mapping threshold is a threshold number of microorganisms with the highest values for a sequencing coverage, based on the mapping of respective nucleotide sequences to the reference sequence of the respective microorganism.

30. The method of any one of claims 1-29, wherein the plurality of mapping statistics includes a count of nucleotide sequences that map to the reference sequence of the respective microorganism.

31. The method of any one of claims 1-30, wherein the plurality of mapping statistics includes a ratio of (i) a count of nucleotide sequences that map to the reference sequence of the respective microorganism and (ii) a total count of nucleotide sequences in the plurality of nucleotide sequences.

32. The method of any one of claims 1-31, wherein the plurality of mapping statistics includes a depth of the mapping of respective nucleotide sequences to the reference sequence of the respective microorganism.

33. The method of any one of claims 1-32, wherein the plurality of mapping statistics includes a consensus sequence for the mapping of respective nucleotide sequences to the reference sequence of the respective microorganism.

34. The method of any one of claims 1-33, wherein the plurality of mapping statistics includes an antimicrobial resistance status detected by:
determining, for the respective microorganism, a locus annotated for antimicrobial resistance; and
when the mapping of the respective nucleotide sequences in the plurality of nucleotide sequences to the reference sequence for the respective microorganism at the respective locus indicates the presence of an antimicrobial resistance marker, including the antimicrobial resistance marker in the subset of the plurality of mapping statistics.

35. The method of claim 34, wherein the customizable user interface further comprises, for each antimicrobial resistance marker in the subset of the plurality of mapping statistics, an affordance for displaying one or more features for the respective antimicrobial resistance marker.

36. The method of any one of claims 1-35, wherein the plurality of sequencing statistics includes, for each sequencing statistic in the plurality of sequencing statistics, a comparison of the respective result set obtained from the respective sequencing reaction to one or more stored result sets.

37. The method of claim 36, wherein the comparison is a distribution.

38. The method of any one of claims 1-37, wherein the plurality of sequencing statistics includes, for each nucleotide base, a count of the respective nucleotide base for each respective nucleotide sequence in the plurality of nucleotide sequences.

39. The method of any one of claims 1-38, wherein a microorganism in the set of at least 3 microorganisms is selected from the group consisting of bacterial, fungal, viral, and parasitic.

40. The method of any one of claims 1-39, wherein a microorganism in the set of at least 3 microorganisms is a pathogen.

41. The method of any one of claims 1-40, wherein the result set further includes a plurality of nucleotide sequences mapped to a human reference genome.

42. The method of any one of claims 1-41, wherein the mapping of the plurality of nucleotide sequences against the reference sequences corresponding to the set of at least 3 microorganisms collectively map to at least 1 megabase of the respective reference sequences.

43. The method of any one of claims 1-42, wherein the plurality of nucleotide sequences includes a first subset of nucleotide sequences that map to a first reference sequence and a second subset of nucleotide sequences that map to a second reference sequence.

44. The method of any one of claims 1-43, wherein the reference sequences of the set of at least 3 microorganisms is at least 3 reference sequences, at least 10 reference sequences, at least 100 reference sequences, at least 1000 reference sequences, at least 1×10⁴ reference sequences, at least 5×10⁴ reference sequences, at least 1×10⁵ reference sequences, at least 1×10⁶ reference sequences, at least 2×10⁶ reference sequences, at least 5×10⁶ reference sequences, or at least 1×10⁷ reference sequences.

45. The method of any one of claims 1-44, wherein the plurality of nucleotide sequences is at least 1×10⁶, at least 1×10⁷, at least 1×10⁸, or at least 2×10⁹ nucleotide sequences.

46. The method of any one of claims 1-45, wherein the sequencing reaction is a whole transcriptome sequencing reaction.

47. The method of any one of claims 1-46, wherein the A) receiving includes:
receiving log-in credentials for a user;
displaying, on the display, a first index of biological or non-biological samples for the user that includes the first biological or non-biological sample; and detecting selection of the first biological or non-biological sample from the index.

48. The method of claim 47, wherein the A) receiving further includes:
displaying, for each respective biological or non-biological sample in the index of biological or non-biological samples for the user, one or more features for the respective biological or non-biological sample selected from the group consisting of:
a review status,
an accession code,
a sample name,
a sample type,
a sample description,
a test profile,
a sample summary,
a run identifier,
a batch identifier,
a run directory,
a run completion time,
an analysis platform version,
a review platform version,
a pipeline version,
an analysis version, and
an analysis completion time.

49. The method of claim 48, wherein the A) receiving further includes:
displaying, for each feature in the one or more features that is displayed, an affordance for applying a filter to the index of biological or non-biological samples, based on an input for the respective feature.

50. The method of any one of claims 47-49, wherein the A) receiving further includes:
displaying, for each respective biological or non-biological sample in the index of biological or non-biological samples for the user, a corresponding sample summary comprising:
an indication of a run quality control metric,
an indication of a sample quality control metric, and
an indication of one or more microorganisms against which nucleotide sequences generated from the respective biological or non-biological sample have been mapped.

51. The method of any one of claims 47-50, wherein the A) receiving includes:
displaying, on the display, an index of sets of biological or non-biological samples for the user that includes a first set comprising the biological or non-biological samples represented in the first index of biological or non-biological samples; and
detecting selection of the first set of biological or non-biological samples from the index,
wherein the displaying of the first index of biological or non-biological samples is responsive to the detecting selection of the first set of biological or non-biological samples.

52. The method of any one of claims 1-51, wherein a mapping statistic in the corresponding plurality of mapping statistics is selected from the group consisting of:
an alert status,
a pathogen status,
an organism name,
an organism class,
an annotation frequency,
a nucleic acid type,
a coverage,
an average nucleotide identity,
a quantity of the nucleic acids from the first biological or non-biological sample,
a length of the reference sequence of the respective microorganism, and
a sequence alignment score.

53. The method of any one of claims 1-52, wherein a sequencing statistic in the plurality of sequencing statistics is selected from the group consisting of:
a total count of nucleotide sequences in the plurality of nucleotide sequences that map to the reference sequences of the set of at least 3 microorganisms,
a count of unique nucleotide sequences in the plurality of nucleotide sequences that map to the reference sequences of the set of at least 3 microorganisms,
a count of nucleotide sequences in the plurality of nucleotide sequences that satisfy a pre-processing criterion,
a quality control metric,
a measure of length for one or more nucleotide sequences in the plurality of nucleotide sequences,
an entropy for one or more nucleotide sequences in the plurality of nucleotide sequences,
a base composition for one or more nucleotide sequences in the plurality of nucleotide sequences,
a size of a sequencing library,
a quantity of a sequencing library, and
an adaptor sequence.

54. The method of any one of claims 1-53, wherein the customizable user interface further comprises a status of an analytical sensitivity based on the mapping of the plurality of nucleotide sequences against the reference sequences of the set of at least 3 microorganisms.

55. The method of any one of claims 1-54, wherein the customizable user interface further comprises an affordance for previewing an exported summary of the analysis of the results set.

56. The method of claim 55, wherein the customizable user interface further comprises an affordance for selecting, for the previewing the exported summary, the subset of the plurality of sequencing statistics and the subset of the plurality of mapping statistics from the results set.

57. The method of any one of claims 1-56, wherein the first biological or non-biological sample is a control sample.

58. The method of any one of claims 1-57, wherein the first biological or non-biological sample is obtained from a biological subject.

59. The method of any one of claims 1-58, wherein the first biological or non-biological sample is obtained from a human with a disease condition.

60. A computer system having a display, one or more processors, and memory storing one or more programs for execution by the one or more processors, the one or more programs including instructions for performing the method of any one of claims 1-59.

61. A computer readable storage medium storing one or more programs, the one or more programs comprising instructions, which, when executed by a computer system having a display and one or more processors, cause the computer system to perform the method of any one of claims 1-59.

62. A graphical user interface on a computer system having a display, one or more processors, and memory storing one or more programs for execution by the one or more processors, the graphical user interface comprising user interfaces displayed in accordance with the method of any one of claims 1-59.
